(19) United States
(12) Patent Application Publication
(10) Pub. No.: US 2007/0129379 A1
(43) Pub. Date: Jun. 7, 2007

(54) HIV INTEGRASE INHIBITORS

(75) Inventors: B. Narasimhulu Naidu, Durham, CT (US); Yasutsugu Ueda, Clinton, CT (US); Timothy P. Connolly, Portland, CT (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/561,039

(22) Filed: Nov. 17, 2006

Related U.S. Application Data
(60) Provisional application No. 60/741,398, filed on Dec. 1, 2005.

(51) Int. Cl. A61K 31/513; C07D 405/02; C07D 403/02

(52) U.S. Cl. 514/255.05; 544/310; 514/269; 544/295; 544/296

(57) ABSTRACT

The invention encompasses a series bicyclic pyrimidinone compounds of Formula I which inhibit HIV integrase and prevent viral integration into human DNA. This action makes the compounds useful for treating HIV infection and AIDS. The invention also encompasses pharmaceutical compositions and methods for treating those infected with HIV.

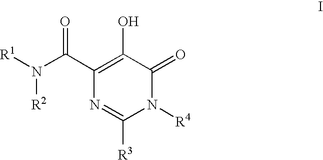

### CROSS REFERENCE TO RELATED APPLICATIONS

This application claims the benefit of U.S. provisional application Ser. No. 60/741,398 filed Dec. 1, 2005.

### BACKGROUND OF THE INVENTION

Human immunodeficiency virus (HIV) has been identified as the etiological agent responsible for acquired immune deficiency syndrome (AIDS), a fatal disease characterized by destruction of the immune system and the inability to fight off life threatening opportunistic infections. Recent statistics (UNAIDS: Report on the Global HIV/AIDS Epidemic, December 1998), indicate that as many as 33 million people worldwide are infected with the virus. In addition to the large number of individuals already infected, the virus continues to spread. Estimates from 1998 point to close to 6 million new infections in that year alone. In the same year there were approximately 2.5 million deaths associated with HIV and AIDS.

There are currently a number of antiviral drugs available to combat the infection. These drugs can be divided into three classes based on the viral protein they target and their mode of action. In particular, saquinavir, indinavir, ritonavir, nelfinavir and amprenavir are competitive inhibitors of the aspartyl protease expressed by HIV. Zidovudine, didanosine, stavudine, lamivudine, zalcitabine and abacavir are nucleoside reverse transcriptase inhibitors that behave as substrate mimics to halt viral cDNA synthesis. The non-nucleoside reverse transcriptase inhibitors, nevaripine, delavirdine and efavirenz inhibit the synthesis of viral cDNA via a non-competitive (or uncompetitive) mechanism. Used alone these drugs are effective in reducing viral replication. The effect is only temporary as the virus readily develops resistance to all known agents. However, combination therapy has proven very effective at both reducing virus and suppressing the emergence of resistance in a number of patients. In the US, where combination therapy is widely available, the number of HIV-related deaths has declined (Palella, F. J.; Delany, K. M.; Moorman, A. C.; Loveless, M. O.; Furher, J.; Satten, G. A.; Aschman, D. J.; Holmberg, S. D. N. Engl. J. Med. 1998, 338, 853-860).

Unfortunately, not all patients are responsive and a large number fail this therapy. In fact, approximately 30-50% of patients ultimately fail combination therapy. Treatment failure in most cases is caused by the emergence of viral resistance. Viral resistance in turn is caused by the rapid turnover of HIV-1 during the course of infection combined with a high viral mutation rate. Under these circumstances incomplete viral suppression caused by insufficient drug potency, poor compliance to the complicated drug regiment as well as intrinsic pharmacological barriers to exposure provides fertile ground for resistance to emerge. More disturbing are recent findings which suggest that low-level replication continues even when viral plasma levels have dropped below detectable levels (<50 copies/ml) (Carpenter, C. C.; Cooper, D. A.; Fischl, M. A.; Gatell, J. M.; Gazzard, B. G.; Hammer, S. M.; Hirsch, M. S.; Jacobsen, D. M.; Katzenstein, D. A.; Montaner, J. S.; Richman, D. D.; Saag, M. S.; Schechter, M.; Schooley, R. T.; Thompson, M. A.; Vella, S.; Yeni, P. G.; Volberding, P. A. JAMA 2000, 283, 381-390). Clearly, there is a need for new antiviral agents, preferably targeting other viral enzymes to reduce the rate of resistance and suppress viral replication even further.

HIV expresses three enzymes, reverse transcriptase, an aspartyl protease, and integrase. All three are targets for treating AIDS and HIV infection. HIV integrase catalyzes insertion of the viral cDNA into the host cell genome, which is a critical step in the viral life cycle. HIV integrase inhibitors belonging to a class of diketo acid compounds prevented viral integration and inhibited HIV-1 replication in cells (Hazuda et al. Science 2000, 287, 646). And recently, HIV integrase inhibitors have been accepted into clinical trials for treating AIDS and HIV infection (Neamati Expert. Opin. Ther. Patents 2002, 12, 709, Pais and Burke Drugs Fut. 2002, 27, 1101).

### DESCRIPTION OF THE INVENTION

The invention encompasses compounds of Formula I, including pharmaceutically acceptable salts, their pharmaceutical compositions, and their use in inhibiting HIV integrase and treating those infected with HIV or AIDS.

One aspect of the invention are compounds of Formula I 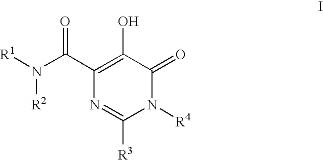
wherein:
R¹ is (Ar¹)alkyl, (Ar¹)(CON(R¹⁰)(R¹¹))alkyl, (Ar¹)(CO₂R¹⁶)alkyl, (Ar¹)hydroxyalkyl, or (Ar¹)oxyalkyl;
R² is hydrogen, alkyl, hydroxy, or alkoxy;
R³ is C(R¹⁷)(R¹⁸)(R¹⁹);
R⁴ is alkyl;
R⁵ is hydrogen, halo, hydroxy, cyano, alkyl, cycloalkyl, C₅₋₇cycloalkenyl, haloalkyl, alkoxy, alkylthio, haloalkoxy, N(R¹⁰)(R¹¹), NHAr², N(R⁸)SO₂R⁹, N(R⁸)COR⁹, N(R⁸)CO₂R⁹, OCOR⁹, OCO₂R⁹, OCON(R¹⁰)(R¹¹), OCH₂CO₂R⁹, OCH₂CON(R¹⁰)(R¹¹), COR⁸, CO₂R⁸, CON(R¹⁰)(R¹¹), SOR⁹, S(═N)R⁹, SO₂R⁹, SO₂N(R⁸)(R⁸), PO(OR⁸)₂, C₂₋₄(R¹⁴)alkynyl, R¹⁵, Ar², or Ar³;
R⁶ is hydrogen, halo, hydroxy, cyano, alkyl, alkoxy, haloalkyl, haloalkoxy, or N(R⁸)(R⁸);
R⁷ is hydrogen, halo, hydroxy, cyano, alkyl, alkoxy, haloalkyl, haloalkoxy, or N(R⁸)(R⁸);
R⁸ is hydrogen, alkyl, or cycloalkyl;
R⁹ is alkyl or cycloalkyl;
R¹⁰ is hydrogen, alkyl, hydroxyalkyl, alkoxyalkyl or dialkylaminoalkyl;
R¹¹ is hydrogen, alkyl, hydroxyalkyl, alkoxyalkyl or dialkylaminoalkyl; or
N(R¹⁰)(R¹¹) taken together is azetidinyl, pyrrolidinyl, (R¹²)-piperidinyl, N—(R¹³)-piperazinyl, morpholinyl, thiomorpholinyl, or dioxothiazinyl;
R¹² is hydrogen, alkyl, or hydroxyalkyl;
R¹³ is hydrogen, alkyl, cycloalkyl, COR⁸, or CO₂R⁸;
R¹⁴ is hydrogen, hydroxy, N(R⁸)(R⁸), SO₂R⁹, OSO₂R⁹, or dioxothiazinyl;
R¹⁵ is azetidinonyl, pyrrolidinonyl, valerolactamyl, caprolactamyl, maleimido, oxazolidonyl, or dioxothiazinyl, and is substituted with 0-1 substituents selected from the group consisting of hydroxymethyl, acetoxymethyl, and aminomethyl;
R¹⁶ is independently hydrogen or alkyl;
or two R¹⁶'s taken together are CH₂CH₂, CH₂CH₂CH₂, CH₂CH₂CH₂CH₂, CH₂CH₂CH₂CH₂CH₂, CH₂CH₂CH₂CH₂CH₂CH₂, OCH₂CH₂, CH₂OCH₂, OCH₂CH₂CH₂, CH₂OCH₂CH₂, OCH₂CH₂CH₂CH₂, CH₂OCH₂CH₂CH₂, CH₂CH₂OCH₂CH₂, OCH₂CH₂CH₂CH₂CH₂, CH₂OCH₂CH₂CH₂CH₂, CH₂CH₂OCH₂CH₂CH₂, N(R⁸)CH₂CH₂, CH₂N(R⁸)CH₂, N(R⁸)CH₂CH₂CH₂, CH₂N(R⁸)CH₂CH₂, N(R⁸)CH₂CH₂CH₂CH₂, CH₂N(R⁸)CH₂CH₂CH₂, CH₂CH₂N(R⁸)CH₂CH₂, N(R⁸)CH₂CH₂CH₂CH₂CH₂, CH₂N(R⁸)CH₂CH₂CH₂CH₂, or CH₂CH₂N(R⁸)CH₂CH₂CH₂, provided that the two R¹⁶'s are attached to a common carbon atom;
R¹⁷ is alkyl;
R¹⁸ is alkyl;
or R¹⁷ and R¹⁸ taken together with the carbon to which they are attached is C₃₋₇cycloalkylene, a 4-7-membered cyclic ether, or a 4-7-membered cyclic thioether;
R¹⁹ is alkyl, hydroxyalkyl, alkylthioalkyl, alkoxy, alkoxyalkoxy, or alkylthioalkoxy; 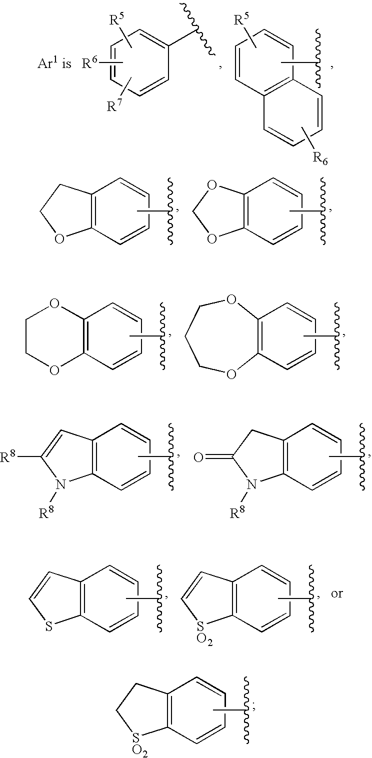
Ar² is tetrazolyl, triazolyl, oxadiazolyl, thiadiazolyl, pyrazolyl, imidazolyl, oxazolyl, thiazolyl, isoxazolyl, isothiazolyl, furanyl, thienyl, pyrrolyl, pyrimidinyl, pyrazinyl, pyridinyl, hydroxypyridinyl, quinolinyl, isoquinolinyl, or indolyl, and is substituted with 0-2 substituents selected from the group consisting of halo, cyano, benzyl, alkyl, alkoxy, N(R¹⁰)(R¹¹), CON(R¹⁰)(R¹¹), CO₂R⁸, CONHSO₂N(R⁸)(R⁸), CONHSO₂N(R⁸)(phenyl), and CONHSO₂N(R⁸)(halophenyl); and
Ar³ is phenyl substituted with 0-2 substituents selected from the group consisting of halo, cyano, hydroxy, alkyl, alkoxy, alkoxymethyl, haloalkyl, haloalkoxy, N(R¹⁰)(R¹¹), CON(R⁸)(R⁸), and CH₂N(R¹⁰)(R¹¹), or is dioxolanylphenyl;
or a pharmaceutically acceptable salt thereof.

Another aspect of the invention is a compound of Formula I where R¹ is (Ar¹)alkyl.

Another aspect of the invention is a compound of Formula I where R¹ is

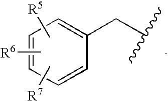

Another aspect of the invention is a compound of Formula I where R¹ is

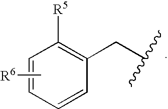

Another aspect of the invention is a compound of Formula I where R¹ is

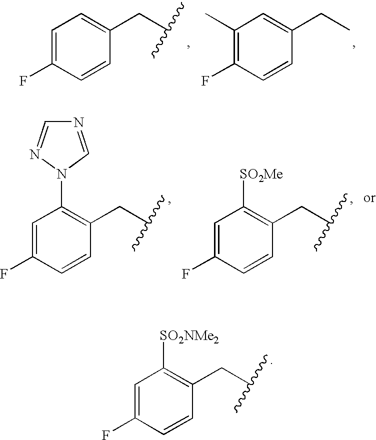

Another aspect of the invention is a compound of Formula I where R² is hydrogen.

Another aspect of the invention is a compound of Formula I where R³ is

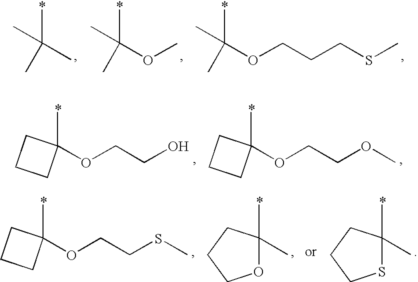

Another aspect of the invention is a compound of Formula I where R⁴ is methyl.

For a compound of Formula I, any scope of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, Ar¹, Ar², and Ar³ can be used independently with any scope of any other substituent. Bach instance of a variable substituent is independent of any other instance.

Unless specified otherwise, these terms have the following meanings. “Alkyl” means a straight or branched alkyl group composed of 1 to 6 carbons. “Alkenyl” means a straight or branched alkyl group composed of 2 to 6 carbons with at least one double bond. “Alkynyl” means a straight or branched alkyl group composed of 2 to 6 carbons with at least one triple bond. “Cycloalkyl” means a monocyclic ring system composed of 3 to 7 carbons. “Haloalkyl” and “haloalkoxy” include all halogenated isomers from monohalo to perhalo. Terms with a hydrocarbon moiety (e.g. alkoxy) include straight and branched isomers for the hydrocarbon portion, “Aryl” includes carbocyclic and heterocyclic aromatic substituents. Parenthetic and multiparenthetic terms are intended to clarify bonding relationships to those skilled in the art. For example, a term such as ((R)alkyl) means an alkyl substituent further substituted with the substituent R.

A “4-7-membered cyclic ether” means a cyclic alkyl ring system where one atom is oxygen. One example is tetrahydrofuranyl.

A “4-7-membered cyclic thioether” means a cyclic alkyl ring system where one atom is sulfur. One example is tetrahydrothiophene.

“(Ar¹)oxyalkyl” means Ar¹ is attached at the oxygen.

“Dioxolanyphenyl” means

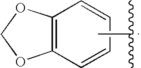

“Dioxothiazinyl” means

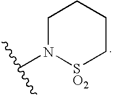

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Some of the compounds of the invention exist in stereoisomeric forms. The invention includes all stereoisomeric forms of the compounds including enantiomers and diastereromers. An example of enantiomers is shown below. Methods of making and separating stereoisomers are known in the art.

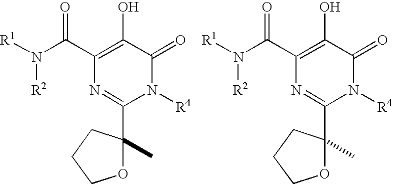

The invention includes all tautomeric forms of the compounds. An example of a tautomeric pair is shown below.

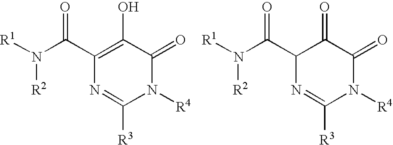

### Synthetic Methods

General methods useful for the synthesis of the compounds of this invention are shown below. Related compounds can be made by reactions known in the art.

Synthetic methods for the preparation of pyrimidines similar to those described in the current invention have been published (Gardelli, C. et al PCT Appl. WO 02/06246). The compounds of the present invention can be synthesized according to Scheme I. In Scheme I, aryl nitrile I-1 is reacted with N-hydroxylamine I-2. The intermediate I-3 generated from this reaction can be isolated but more often is reacted in one pot with dialkyl acetylenedicarboxylate I-4 to yield the diesters I-5a or I-5b. The diesters I-5a or I-5b were converted to pyrimidine carboxylate I-6 by heating at or above 120° C. in an appropriate solvent. The ester I-6 is condensed with amine I-7 to give the amide I-8. The amide coupling reaction can be carried out under a variety of conditions such as those disclosed in Jerry March, Advanced Organic Chemistry, 3^{rd} edition, John Wiley & Sons, 1985.

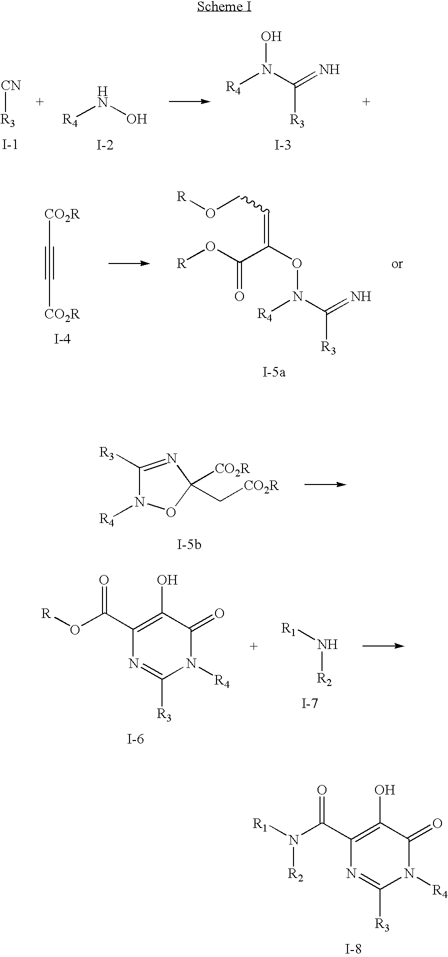

In Scheme II, an alternative pathway is shown in which the R⁶ group is introduced at a later stage of the synthesis. Synthetic methods for the preparation of pyrimidines similar to those described in the current invention have been published (Sunderland, J. S.; et al. Inorg. Chem. (2001), 40, 6756-6756). The compounds of the present invention can be synthesized according to Scheme II. In Scheme II, an oxalic acid diester II-1 is condensed with glycolate II-2 using sodium hydride or a similar base. The intermediate II-3 generated from this reaction can be isolated but more often is reacted in one pot with an appropriately substituted amidine II-4 to yield the pyrimidinone heterocycle II-5. Intermediate II-5 is coupled with amine II-6. Alternatively, the pyrimidinone II-5 is alkylated with a suitable electrophile under basic conditions. Then intermediate II-9 is coupled with amine II-6. The amides, II-8 and II-10 are then treated under conditions appropriate for cleaving the protecting group P. For alkyl groups, where P is alkyl, this can be accomplished by BBr₃ or other conditions known in the art. Alternatively, when P is a benzylic or substituted benzylic group the ether can be cleaved under reductive conditions, oxidative conditions or acidic conditions. Protecting groups, R and P, useful for the synthesis of compounds such as 1-9 can be found in Greene, T. W. and Wutz, P. G. M. Protective Groups in Organic Synthesis, Second Edition, 1991, John Wiley and Sons, New York.

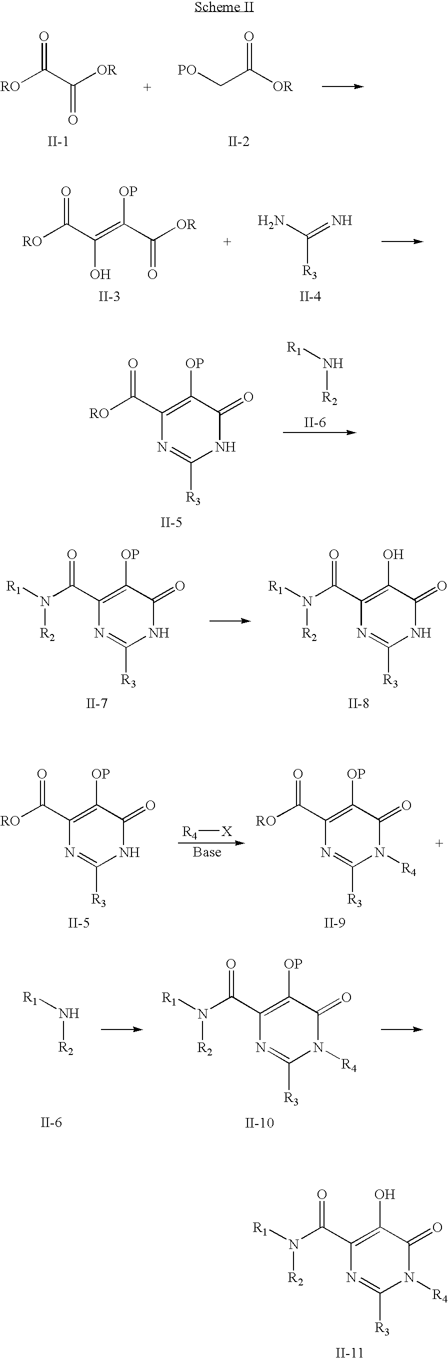

### Biological Methods

HIV-Integrase Inhibition Activity. To evaluate in-vitro activity against HIV-integrase, 5 pmole of biotin labeled substrate DNA was bound to 100 μg of Streptavidin coated PVT SPA beads (Amersham Pharmacia Biotech). Recombinant integrase (0.26 ng) was incubated with the beads for 90 min at 37° C. Unbound enzyme was removed by washing the complex followed by addition of inhibitors and 0.1 fmol of P33 labeled target DNA. The reaction was stopped by adding EDTA to a final concentration of 10 mM. Samples were counted in TopCountNXT (Packard) and the CPM was used as a measure of integration. The reaction condition was as described in A. Engleman and R. Craige, J. Virol. 69, 5908-5911 (1995). The sequences of substrate and target DNA were described in Nucleic Acid Research 22, 1121-1122 (1994). Results are shown in the Table 1. Activity equal to A refers to a compound having IC₅₀=0.002 to 0.10 μM while B and C denote compounds having IC₅₀=0.1 to 1.0 μM and IC₅₀≧1.0 μM respectively.

TABLE 1

| Example | Activity |
|---|---|
| 1 | A |
| 2 | A |
| 3 | A |
| 4 | A |
| 5 | A |
| 6 | A |
| 7 | A |
| 8 | A |
| 9 | A |
| 10 | A |
| 11 | A |
| 12 | A |
| 13 | A |
| 14 | A |
| 15 | A |
| 16 | A |
| 17 | A |
| 18 | A |

Inhibition of HIV replication. A recombinant NL-Rluc virus was constructed in which a section of the nef gene from NL4-3 was replaced with the Renilla Luciferase gene. The NL-RLuc virus was prepared by co-transfection of two plasmids, pNLRLuc and pVSVenv. The pNLRLuc contains the NL-Rluc DNA cloned into pUC18 at the PvuII site, while the pVSVenv contains the gene for VSV G protein linked to an LTR promoter. Transfections were performed at a 1:3 ratio of pNLRLuc to pVSVenv on 293T cells using the LipofectAMINE PLUS kit from Invitrogen (Carlsbad, Calif.) according to manufactures instruction, and the pseudotype virus generated was titered in MT-2 cells.

Susceptibility of viruses to compounds was determined by incubation in the presence of serial dilutions of the compound. The 50% effective concentration (EC₅₀) was calculated by using the exponential form of the median effect equation where (Fa)=1/[1+(ED₅₀/drug conc.)^{m}] (Johnson V A, Byington R T. Infectivity Assay. In Techniques in HIV Research. ed. Aldovini A, Walker B D. 71-76. New York: Stockton Press. 1990). The anti-viral activity of compounds was evaluated under three serum conditions, 10% FBS, 15 mg/ml human serum albumin 10% FBS or 40% human serum/5% FBS, and the results from at least 2 experiments were used to calculate the ED₅₀ values. Results are shown in the Table 2. Activity equal to A refers to a compound having EC₅₀=0.003 to 0.10 μM while B and C denote compounds with EC₅₀=0.1 to 1.0 μM and EC₅₀≧1.0 μM respectively.

TABLE 2

| Example | Activity |
|---|---|
| 1 | A |
| 2 | A |
| 3 | A |
| 4 | A |
| 5 | A |
| 6 | A |
| 7 | A |
| 8 | A |
| 9 | A |
| 10 | A |
| 11 | A |
| 12 | A |
| 13 | A |
| 14 | A |
| 15 | A |
| 16 | A |
| 17 | A |
| 18 | A |

See US 20050250256109 and US 20050267105 for some other compounds demonstrating HIV integrase activity.

### Pharmaceutical Composition and Methods of Use

The compounds of this invention inhibit HIV integrase. HIV integrase inhibitors belonging to a class of diketo acid compounds prevented viral integration and inhibited HIV-1 replication in cells (Hazuda et al. Science 2000, 287, 646). Recently, HIV integrase inhibitors have been accepted into clinical trials for treating AIDS and HIV infection (Neamati Expert. Opin. Ther. Patents 2002, 12, 709, Pais and Burke Drugs Fut. 2002, 27, 1101).

Accordingly, another aspect of the invention is a method for treating HIV infection in a human patient comprising administering a therapeutically effective amount of a compound of Formula 1, or a pharmaceutically acceptable salt thereof, with a pharmaceutically acceptable carrier.

Another aspect of the invention is a method for treating HIV infection in a human patient comprising the administration of a therapeutically effective amount of a compound of Formula I, or a pharmaceutically acceptable salt thereof, with a therapeutically effective amount of at least one other agent used for treatment of AIDS or HIV infection selected from the group consisting of nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

Another aspect of the invention is a method wherein the agent is a nucleoside HIV reverse transcriptase inhibitor.

Another aspect of the invention is a method wherein the nucleoside HIV reverse transcriptase inhibitor is selected from the group consisting of abacavir, didanosine, emtricitabine, lamivudine, stavudine, tenofovir, zalcitabine, and zidovudine, or a pharmaceutically acceptable salt thereof.

Another aspect of the invention is a method wherein the agent is a non-nucleoside HIV reverse transcriptase inhibitor.

Another aspect of the invention is a method wherein the non-nucleoside HIV reverse transcriptase inhibitor is selected from the group consisting of delavirdine, efavirenz, and nevirapine, or a pharmaceutically acceptable salt thereof.

Another aspect of the invention is a method wherein the agent is an HIV protease inhibitor.

Another aspect of the invention is a method wherein the HIV protease inhibitor is selected from the group consisting of amprenavir, atazanavir, indinavir, lopinavir, nelfinavir, ritonavir, saquinavir and fosamprenavir, or a pharmaceutically acceptable salt thereof.

Another aspect of the invention is a method wherein the agent is an HIV fusion inhibitor.

Another aspect of the invention is a method wherein the HIV fusion inhibitor is enfuvirtide or T-1249, or a pharmaceutically acceptable salt thereof.

Another aspect of the invention is a method wherein the agent is an HIV attachment inhibitor.

Another aspect of the invention is a method wherein the agent is a CCR5 inhibitor.

Another aspect of the invention is a method wherein the CCR5 inhibitor is selected from the group consisting of Sch-C, Sch-D, TAK-220, PRO-140, and UK-427,857, or a pharmaceutically acceptable salt thereof.

Another aspect of the invention is a method wherein the agent is a CXCR4 inhibitor.

Another aspect of the invention is a method wherein the CXCR4 inhibitor is AMD-3100, or a pharmaceutically acceptable salt thereof.

Another aspect of the invention is a method wherein the agent is an HIV budding or maturation inhibitor.

Another aspect of the invention is a method wherein the budding or maturation inhibitor is PA-457, or a pharmaceutically acceptable salt thereof.

Another aspect of the invention is a method wherein the agent is an HIV integrase inhibitor.

Another aspect of the invention is a pharmaceutical composition comprising a therapeutically effective amount of a compound of Formula I, or a pharmaceutically acceptable salt thereof, with at least one other agent used for treatment of AIDS or HIV infection selected from the group consisting of nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier.

Another aspect of the invention is the composition wherein the agent is a nucleoside HIV reverse transcriptase inhibitor.

Another aspect of the invention is the composition wherein the nucleoside HIV transcriptase inhibitor is selected from the group consisting of abacavir, didanosine, emtricitabine, lamivudine, stavudine, tenofovir, zalcitabine, and zidovudine, or a pharmaceutically acceptable salt thereof.

Another aspect of the invention is the composition wherein the agent is a non nucleoside HIV reverse transcriptase inhibitor.

Another aspect of the invention is the composition wherein the non-nucleoside HIV reverse transcriptase inhibitor is selected from the group consisting of delavirdine, efavirenz, and nevirapine, or a pharmaceutically acceptable salt thereof.

Another aspect of the invention is the composition wherein the agent is an HIV protease inhibitor.

Another aspect of the invention is the composition wherein the HIV protease inhibitor is selected from the group consisting of amprenavir, atazanavir, indinavir, lopinavir, nelfinavir, ritonavir, saquinavir and fosamprenavir, or a pharmaceutically acceptable salt thereof.

Another aspect of the invention is the composition wherein the agent is an HIV fusion inhibitor.

Another aspect of the invention is the composition method wherein the HIV fusion inhibitor is enfuvirtide or T-1249, or a pharmaceutically acceptable salt thereof.

Another aspect of the invention is the composition wherein the agent is an HIV attachment inhibitor.

Another aspect of the invention is the composition wherein the agent is a CCR5 inhibitor.

Another aspect of the invention is the composition wherein the CCR5 inhibitor is selected from the group consisting of Sch-C, Sch-D, TAK-220, PRO-140, and UK-427,857, or a pharmaceutically acceptable salt thereof.

Another aspect of the invention is a method wherein the agent is a CXCR4 inhibitor.

Another aspect of the invention is a method wherein the CXCR4 inhibitor is AMD-3100 or a pharmaceutically acceptable salt thereof.

Another aspect of the invention is the composition wherein the agent is an HIV budding or maturation inhibitor.

Another aspect of the invention is the composition wherein the budding or maturation inhibitor is PA-457, or a pharmaceutically acceptable salt thereof.

Another aspect of the invention is the composition wherein the agent is an HIV integrase inhibitor.

“Combination,” “coadministration,” “concurrent,” and similar terms referring to the administration of a compound of Formula I with at least one anti-HIV agent mean that the components are part of a combination antiretroviral therapy or highly active antiretroviral therapy (HAART) as understood by practitioners in the field of AIDS and HIV infection.

“Therapeutically effective” means the amount of agent required to provide a meaningful patient benefit as understood by practitioners in the field of AIDS and HIV infection. In general, the goals of treatment are suppression of viral load, restoration and preservation of immunologic function, improved quality of life, and reduction of HIV-related morbidity and mortality.

“Patient” means a person infected with the HIV virus and suitable for therapy as understood by practitioners in the field of AIDS and HIV infection.

“Treatment,” “therapy,” “regimen,” “HIV infection,” “ARC,” “AIDS” and related terms are used as understood by practitioners in the field of AIDS and HIV infection.

The compounds of this invention are generally given as pharmaceutical compositions comprised of a therapeutically effective amount of a compound of Formula I or its pharmaceutically acceptable salt and a pharmaceutically acceptable carrier and may contain conventional excipients. A therapeutically effective amount is that which is needed to provide a meaningful patient benefit. Pharmaceutically acceptable carriers are those conventionally shown carriers having acceptable safety profiles. Compositions encompass all common solid and liquid forms including capsules, tablets, losenges, and powders as well as liquid suspensions, syrups, elixers, and solutions. Compositions are made using common formulation techniques, and conventional excipients (such as binding and wetting agents) and vehicles (such as water and alcohols) are generally used for compositions.

Solid compositions are normally formulated in dosage units and compositions providing from about 1 to 1000 mg of the active ingredient per dose are preferred. Some examples of dosages are 1 mg, 10 mg, 100 mg, 250 mg, 500 mg, and 1000 mg. Generally, other antiretroviral agents will be present in a unit range similar to agents of that class used clinically. Typically, this is 0.25-1000 mg/unit.

Liquid compositions are usually in dosage unit ranges. Generally, the liquid composition will be in a unit dosage range of 1-100 mg/mL. Some examples of dosages are 1 mg/mL, 10 mg/mL, 25 mg/mL, 50 mg/mL, and 100 mg/mL. Generally, other antiretroviral agents will be present in a unit range similar to agents of that class used clinically. Typically, this is 1-100 mg/mL.

The invention encompasses all conventional modes of administration; oral and parenteral methods are preferred. Generally, the dosing regimen will be similar to other antiretroviral agents used clinically. Typically, the daily dose will be 1-100 mg/kg body weight daily. Generally, more compound is required orally and less parenterally. The specific dosing regime, however, will be determined by a physician using sound medical judgement.

The invention also encompasses methods where the compound is given in combination therapy. That is, the compound can be used in conjunction with, but separately from, other agents useful in treating AIDS and HIV infection. Some of these agents include HIV attachment inhibitors, CCR5 inhibitors, CXCR4 inhibitors, HIV cell fusion inhibitors, HIV integrase inhibitors, HIV nucleoside reverse transcriptase inhibitors, HIV non-nucleoside reverse transcriptase inhibitors, HIV protease inhibitors, budding and maturation inhibitors, immunomodulators, and anti-infectives. In these combination methods, the compound of Formula I will generally be given in a daily dose of 1-100 mg/kg body weight daily in conjunction with other agents. The other agents generally will be given in the amounts used therapeutically. The specific dosing regime, however, will be determined by a physician using sound medical judgement.

Table 4 lists some agents useful in treating AIDS and HIV infection which are suitable for this invention.

TABLE 4

| | | |
|---|---|---|
| DRUG NAME | MANUFACTURER | INDICATION |

ANTIVIRALS

| | | |
|---|---|---|
| 097 | Hoechst/Bayer | HIV infection, AIDS, |
| (non-nucleoside | | ARC |
| reverse transcrip- | | |
| tase inhibitor) | | |
| Amprenavir | Glaxo Wellcome | HIV infection, AIDS, |
| 141 W94 | | ARC |
| GW 141 | | |
| (protease inhibitor) | | |
| Abacavir (1592U89) | Glaxo Wellcome | HIV infection, AIDS, |
| GW 1592 | | ARC |
| (RT inhibitor) | | |
| Acemannan | Carrington Labs | ARC |
| | (Irving, TX) | |
| Acyclovir | Burroughs Wellcome | HIV infection, AIDS, |
| | | ARC, in combination |
| | | with AZT |
| AD-439 | Tanox Biosystems | HIV infection, AIDS, |
| | | ARC |
| AD-519 | Tanox Biosystems | HIV infection, AIDS, |
| | | ARC |
| Adefovir dipivoxil | Gilead Sciences | HIV infection, ARC, |
| AL-721 | Ethigen | PGL HIV positive, |
| | (Los Angeles, CA) | AIDS |
| Alpha Interferon | Glaxo Wellcome | Kaposi's sarcoma |
| HIV in combination | | |
| w/Retrovir | | |
| Ansamycin | Adria Laboratories | ARC |
| LM 427 | (Dublin, OH) | |
| | Erbamont | |
| | (Stamford, CT) | |
| Antibody which | Advanced Biotherapy | AIDS, ARC |
| Neutralizes pH | Concepts | |
| Labile alpha | (Rockville, MD) | |
| aberrant | | |
| Interferon | | |
| AR177 | Aronex Pharm | HIV infection, AIDS, |
| | | ARC |
| Beta-fluoro-ddA | Nat'l Cancer | AIDS-associated |
| | Institute | diseases |
| BMS-232623 | Bristol-Myers | HIV infection, AIDS, |
| (CGP-73547) | Squibb/Novartis | ARC |
| (protease inhibitor) | | |
| BMS-234475 | Bristol-Myers | HIV infection, AIDS, |
| (CGP-61755) | Squibb/Novartis | ARC |
| (protease inhibitor) | | |
| CI-1012 | Warner-Lambert | HIV-1 infection |
| Cidofovir | Gilead Science | CMV retinitis, herpes, |
| | | papillomavirus |
| Curdlan sulfate | AJI Pharma USA | HIV infection |
| Cytomegalovirus | MedImmune | CMV retinitis |
| Immune globin | | |
| Cytovene | Syntex | Sight threatening |
| Ganciclovir | | CMV peripheral, CMV |
| | | retinitis |
| Delaviridine | Pharmacia-Upjohn | HIV infection, AIDS, |
| (RT inhibitor) | | ARC |
| Dextran Sulfate | Ueno Fine Chem. | AIDS, ARC, HIV |
| | Ind. Ltd. (Osaka, | positive asymptomatic |
| | Japan) | |
| ddC | Hoffman-La Roche | HIV infection, AIDS, |
| Dideoxycytidine | | ARC |
| ddI | Bristol-Myers | HIV infection, AIDS, |
| Dideoxyinosine | Squibb | ARC; combination with |
| | | AZT/d4T |
| DMP-450 | AVID | HIV infection, AIDS, |
| (protease inhibitor) | (Camden, NJ) | ARC |
| Efavirenz | DuPont Merck | HIV infection, AIDS, |
| (DMP 266) | | ARC |
| (−)6-Chloro-4-(S)- | | |
| cyclopropylethynyl- | | |
| 4(S)-trifluoro- | | |
| methyl-1,4-dihydro- | | |
| 2H-3,1-benzoxazin- | | |
| 2-one, STOCRINE | | |
| (non-nucleoside RT | | |
| inhibitor) | | |
| EL10 | Elan Corp, PLC | HIV infection |
| | (Gainesville, GA) | |
| Famciclovir | Smith Kline | herpes zoster, herpes |
| | | simplex |
| FTC | Emory University | HIV infection, AIDS, |
| (reverse transcrip- | | ARC |
| tase inhibitor) | | |
| GS 840 | Gilead | HIV infection, AIDS, |
| (reverse transcrip- | | ARC |
| tase inhibitor) | | |
| HBY097 | Hoechst Marion | HIV infection, AIDS, |
| (non-nucleoside | Roussel | ARC |
| reverse transcrip- | | |
| taseinhibitor) | | |
| Hypericin | VIMRx Pharm. | HIV infection, AIDS, |
| | | ARC |
| Recombinant Human | Triton Biosciences | AIDS, Kaposi's |
| Interferon Beta | (Almeda, CA) | sarcoma, ARC |
| Interferon alfa-n3 | Interferon | ARC, AIDS |
| | Sciences | |
| Indinavir | Merck | HIV infection, AIDS, |
| | | ARC, asymptomatic HIV |
| | | positive, also in |
| | | combination with |
| | | AZT/ddI/ddC |
| ISIS 2922 | ISIS Pharma- | CMV retinitis |
| | ceuticals | |
| KNI-272 | Nat'l Cancer | HIV-associated |
| | Institute | diseases |
| Lamivudine, 3TC | Glaxo Wellcome | HIV infection, AIDS, |
| (reverse transcrip- | | ARC, also with AZT |
| tase inhibitor) | | |
| Lobucavir | Bristol-Myers | CMV infection |
| | Squibb | |
| Nelfinavir | Agouron | HIV infection, AIDS, |
| (protease inhibitor) | Pharmaceuticals | ARC |
| Nevirapine | Boeheringer | HIV infection, AIDS, |
| (RT inhibitor) | Ingleheim | ARC |
| Novapren | Novaferon Labs, | HIV inhibitor |
| | Inc. (Akron, OH) | |
| Peptide T | Peninsula Labs | AIDS |
| Octapeptide | (Belmont, CA) | |
| Sequence | | |
| Trisodium | Astra Pharm. | CMV retinitis, HIV |
| Phosphonoformate | Products, Inc. | infection, other CMV |
| | | infections |
| PNU-140690 | Pharmacia Upjohn | HIV infection, AIDS, |
| (protease inhibitor) | | ARC |
| Probucol | Vyrex | HIV infection, AIDS |
| RBC-CD4 | Sheffield Med. | HIV infection, AIDS, |
| | Tech (Houston, TX) | ARC |
| Ritonavir | Abbott | HIV infection, AIDS, |
| (protease inhibitor) | | ARC |
| Saquinavir | Hoffmann- | HIV infection, AIDS, |
| (protease inhibitor) | LaRoche | ARC |
| Stavudine; d4T | Bristol-Myers | HIV infection, AIDS, |
| Didehydrodeoxy- | Squibb | ARC |
| thymidine | | |
| Valaciclovir | Glaxo Wellcome | Genital HSV & |
| | | CMV infections |
| Virazole | Viratek/ICN | asymptomatic HIV- |
| Ribavirin | (Costa Mesa, CA) | positive, LAS, ARC |
| VX-478 | Vertex | HIV infection, AIDS, |
| | | ARC |
| Zalcitabine | Hoffmann-LaRoche | HIV infection, AIDS, |
| | | ARC, with AZT |
| Zidovudine; AZT | Glaxo Wellcome | HIV infection, AIDS, |
| | | ARC, Kaposi's sarcoma, |
| | | in combination with |
| | | other therapies |
| Tenofovir diso- | Gilead | HIV infection, AIDS |
| proxil, fumarate | | |
| salt (Viread ®) | | |
| (reverse transcrip- | | |
| tase inhibitor) | | |
| Combivir ® | GSK | HIV infection, AIDS |
| (reverse transcrip- | | |
| tase inhibitor) | | |
| abacavir succinate | GSK | HIV infection, AIDS |
| (or Ziagen ®) | | |
| (reverse transcrip- | | |
| tase inhibitor) | | |
| Reyataz ® | Bristol-Myers | HIV infection, AIDS |
| (atazanavir) | Squibb | |
| Fuzeon | Roche/Trimeris | HIV infection, AIDS, |
| (Enfuvirtide, T-20) | | viral fusion inhibitor |
| Trizivir ® | | HIV infection, AIDS |
| Kaletra ® | Abbott | HIV infection, AIDS, |
| | | ARC |

IMMUNOMODULATORS

| | | |
|---|---|---|
| AS-101 | Wyeth-Ayerst | AIDS |
| Bropirimine | Pharmacia Upjohn | Advanced AIDS |
| Acemannan | Carrington Labs, | AIDS, ARC |
| | Inc. (Irving, TX) | |
| CL246, 738 | American Cyanamid | AIDS, Kaposi's sarcoma |
| | Lederle Labs | |
| EL10 | Elan Corp, PLC | HIV infection |
| | (Gainesville, GA) | |
| FP-21399 | Fuki ImmunoPharm | Blocks HIV fusion with |
| | | CD4+ cells |
| Gamma Interferon | Genentech | ARC, in combination |
| | | w/TNF (tumor necrosis |
| | | factor) |
| Granulocyte | Genetics Institute | AIDS |
| Macrophage Colony | Sandoz | |
| Stimulating Factor | | |
| Granulocyte | Hoechst-Roussel | AIDS |
| Macrophage Colony | Immunex | |
| Stimulating Factor | | |
| Granulocyte | Schering-Plough | AIDS, combination |
| Macrophage Colony | | w/AZT |
| Stimulating Factor | | |
| HIV Core Particle | Rorer | Seropositive HIV |
| Immunostimulant | | |
| IL-2 | Cetus | AIDS, in combination |
| Interleukin-2 | | w/AZT |
| IL-2 | Hoffman-LaRoche | AIDS, ARC, HIV, in |
| Interleukin-2 | Immunex | combination w/AZT |
| IL-2 | Chiron | AIDS, increase in CD4 |
| Interleukin-2 | | cell counts |
| (aldeslukin) | | |
| Immune Globulin | Cutter Biological | Pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's |
| | (New Orleans, LA) | sarcoma, ARC, PGL |
| Imuthiol Diethyl | Merieux Institute | AIDS, ARC |
| Dithio Carbamate | | |
| Alpha-2 | Schering Plough | Kaposi's sarcoma |
| Interferon | | w/AZT, AIDS |
| Methionine- | TNI Pharmaceutical | AIDS, ARC |
| Enkephalin | (Chicago, IL) | |
| MTP-PE | Ciba-Geigy Corp. | Kaposi's sarcoma AIDS, |
| Muramyl-Tripeptide | Amgen | in combination w/AZT |
| Granulocyte | | |
| Colony Stimulating | | |
| Factor | | |
| Remune | Immune Response | Immunotherapeutic |
| | Corp. | |
| rCD4 | Genentech | AIDS, ARC |
| Recombinant | | |
| Soluble Human CD4 | | |
| rCD4-IgG | | AIDS, ARC |
| hybrids | | |
| Recombinant | Biogen | AIDS, ARC |
| Soluble Human CD4 | | |
| Interferon | Hoffman-La Roche | Kaposi's sarcoma, |
| Alfa 2a | in combination | AIDS, ARC |
| | w/AZT | |
| SK&F106528 | Smith Kline | HIV infection |
| Soluble T4 | | |
| Thymopentin | Immunobiology | HIV infection |
| | Research Institute | |
| | (Annandale, NJ) | |
| Tumor Necrosis | Genentech | ARC, in combination |
| Factor; TNF | | w/gamma Interferon |

ANTI-INFECTIVES

| | | |
|---|---|---|
| Clindamycin with | Pharmacia Upjohn | PCP |
| Primaquine | | |
| Fluconazole | Pfizer | Cryptococcal |
| | | meningitis, |
| | | candidiasis |
| Pastille | Squibb Corp. | Prevention of oral |
| Nystatin Pastille | | candidiasis |
| Ornidyl | Merrell Dow | PCP |
| Eflornithine | | |
| Pentamidine | LyphoMed | PCP treatment |
| Isethionate | (Rosemont, IL) | |
| (IM & IV) | | |
| Trimethoprim | | Antibacterial |
| Trimethoprim/sulfa | | Antibacterial |
| Piritrexim | Burroughs Wellcome | PCP treatment |
| Pentamidine | Fisons Corporation | PCP prophylaxis |
| Isethionate for | | |
| Inhalation | | |
| Spiramycin | Rhone-Poulenc | Cryptosporidial |
| | diarrhea | |
| Intraconazole- | Janssen-Pharm. | Histoplasmosis; |
| R51211 | | cryptococcal |
| | | meningitis |
| Trimetrexate | Warner-Lambert | PCP |
| Daunorubicin | NeXstar, Sequus | Kaposi's sarcoma |
| Recombinant Human | Ortho Pharm. Corp. | Severe anemia assoc. |
| Erythropoietin | | with AZT therapy |
| Recombinant Human | Serono | AIDS-related wasting, |
| Growth Hormone | | cachexia |
| Megestrol Acetate | Bristol-Myers | Treatment of anorexia |
| | Squibb | assoc. W/AIDS |
| Testosterone | Alza, Smith Kline | AIDS-related wasting |
| Total Enteral | Norwich Eaton | Diarrhea and |
| Nutrition | Pharmaceuticals | malabsorption |
| | | related to AIDS |

### DESCRIPTION OF SPECIFIC EMBODIMENTS

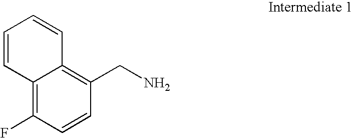

(4-Fluoronaphthalen-1-yl)methanamine hydrochloride. A solution of 1-cyano-4-fluoronapthalene (1.05 g, 6.12 mmol) and 1.5 mL of HCl (aq.) in absolute ethanol (50 mL) was stirred under a hydrogen atmosphere (balloon) with 10% palladium on carbon (0.20 g) for 16 hours. The catalyst was removed by filtration through Celite, and the filtrate concentrated under vacuum. The resulting solid was triturated with ether and collected by filtration to give the title compound (0.575 g, 44% yield) as an off white solid.

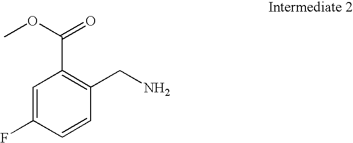

Methyl 2-(aminomethyl)-5-fluorobenzoate trifluoroacetic acid salt. Methyl 2-((tert-butoxycarbonyl)methyl)-5-fluorobenzoate, prepared according to literature methods, was treated with trifluoroacetic acid to provide the title compound. Yield 100%; ¹H NMR (300 MHz, DMSO-d6) δ ppm: 3.89 (3H, s) 4.32 (2H, q, J=5.61 Hz) 7.51-7.71 (2H, m) 7.78 (1H, dd, J=9.33, 2.38 Hz) 8.13 (2H, brs); LC/MS m/z 184 (M+H)

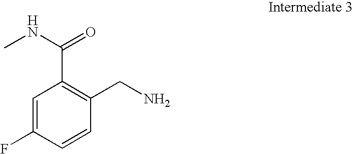

2-Aminomethyl-5-fluoro-N-methyl-benzamide trifluoroacetic acid salt. To a solution of tert-butyl 4-fluoro-2-(methylcarbamoyl)benzylcarbamate (7.70 g, 27.3 mmol; prepared from 2-bromo-5-fluorobenzoic acid using literature methods) in CH₂Cl₂ (100 mL) was added CF₃CO₂H (25 mL) and the mixture stirred at room temperature for 15 min. This was concentrated in vacuo and the residue triturated with diethyl ether to obtain 8.0 g (Yield 99%) of the title compound as a white powder. ¹H NMR (300 MHz, D₂O) δ ppm: 2.93 (3H, s) 4.20 (2H, s) 7.35 (1H, dt, J=8.5, 3 Hz) 7.42 (1H, dd, J=9.0, 2.7 Hz) 7.57 (1H, dd, J=8.4, 5.5 Hz); LC/MS m/z 183 (M+H).

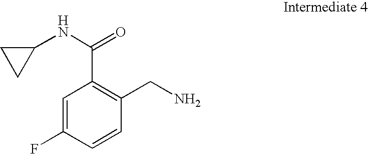

2-(Aminomethyl)-N-cyclopropyl-5-fluorobenzamide trifluoroacetic acid salt. A solution of tert-butyl 2-(cyclopropylcarbamoyl)-4-fluorobenzylcarbamate (130 mg, 0.42 mmol) prepared according to literature methods, in CH₂Cl₂ (5 mL) was stirred with trifluoroacetic acid (3 mL) at room temperature for 10 min, then concentrated in vacuo to give 140 mg (Yield 100%) of the title compound as a foam: ¹H NMR (DMSO-d6, 300 MHz) δ ppm: 0.62 (2H, m, CH₂), 0.73 (2H, m, CH₂), 2.86 (1H, m, CH), 4.02-4.07 (2H, ABq, NCH₂), 7.46 (2H, m, Ar—Hs), 7.58 (1H, m, Ar—H), 8.11 (3H, br, NH3), 8.81 (1H, d, J=4.4 Hz, NH); LC/MS m/z 209 (M+H).

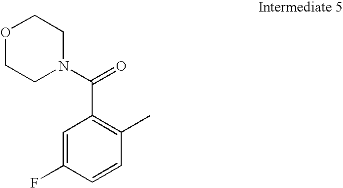

(5-Fluoro-2-methylphenyl)(morpholino)methanone. To a solution of morpholine (870 mg, 10 mmol) and triethylamine (1.1 g, 10.8 mmol) in CH₂Cl₂ (15 mL) was added a solution of 5-fluoro-2-methylbenzoyl chloride (1.72 g, 10 mmol) in CH₂Cl₂ (5 mL), dropwise, and the mixture stirred for 15 min. The mixture was then washed with water, and the organic phase dried (MgSO₄), filtered, and concentrated to obtain 2.19 g (Yield 98%) of the title compound as a solid: ¹H NMR (500 MHz, CDCl₃) δ ppm: 2.27 (3H, s) 3.24 (2H, d, J=4 Hz) 3.58 (2H, s) 3.79 (4H, dd, J=18, 3.8 Hz) 6.88 (1H, dd, J=8.2, 2.8 Hz) 6.92-7.05 (1H, m) 7.18 (1H, dd, J=8.4, 5.3 Hz).

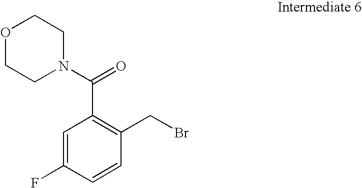

(2-(Bromomethyl)-5-fluorophenyl)(morpholino)methanone. A mixture of intermediate 41, (5-fluoro-2-methylphenyl)(morpholino)methanone, (2.1 g, 9.5 mmol) and N-bromosuccinimide (2.0 g, 11 mmol) in CCl₄ (30 mL) was heated at reflux. To this mixture was added benzoylperoxide (242 mg, 1 mmol) and the mixture heated at reflux for 2 hrs. After cooling, the insoluble materials were filtered and the filtrate purified by column chromatography (SiO₂, 0-10% ether in CH₂Cl₂) to give 1.1 g (Yield 38%) of the title compound as a clear oil: ¹H NMR (300 MHz, CDCl₃) δ ppm: 3.31 (2H, t, J=4.94 Hz) 3.55-4.02 (6H, m) 4.56 (2H, dd, J=128.81, 9.51 Hz) 6.89 (1H, dd, J=8.23, 2.74 Hz) 6.96-7.12 (1H, m) 7.33-7.49 (1H, m); LC/MS m/z 302 (M+H).

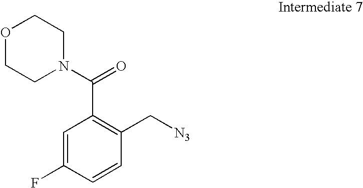

(2-(Azidomethyl)-5-fluorophenyl)(morpholino)methanone. To a solution of intermediate 42, (2-(bromomethyl)-5-fluorophenyl)(morpholino)methanone, (1.0 g, 3.32 mmol) in dimethylformamide (10 mL) was added sodium azide (230 mg, 3.5 mmol) and the mixture stirred under a nitrogen atmosphere for 1 h. The solvent was evaporated in vacuo, and the residue dissolved in CH₂Cl₂, then washed with water. The organic phase was dried (Na₂SO₄), filtered, concentrated, and the residue purified by column chromatography (SiO₂, CH₂Cl₂) to provide 770 mg (Yield 88%) of the title compound as an oil: ¹H NMR (300 MHz, CDCl₃) δ ppm: 3.27 (2H, s) 3.51-3.65 (2H, m) 3.66-3.97 (4H, m) 4.38 (2H, brs) 6.92 (1H, dd, J=8.2, 2.7 Hz) 7.07 (1H, dt, J=8.5, 3 Hz) 7.34 (1H, dd, J=8.4, 5.5 Hz); LC/MS m/z 265 (M+H).

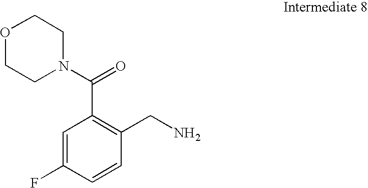

(2-(Aminomethyl)-5-fluorophenyl)(morpholino)methanone hydrochloride. To a solution of intermediate 43, (2-(azidomethyl)-5-fluorophenyl)(morpholino)methanone, (770 mg, 2.92 mmol) in ethanol (20 mL) was added 4N HCl (1 mL) and 10% Pd—C (100 mg), and the mixture hydrogenated at 1 atm of H₂ for 3 hrs. The catalyst was removed by filtration and the filtrate concentrated. The residue was purified by C18 reverse phase silica gel column chromatography (YMC ODS, 0-5% CH₃CN/H₂O) to obtain 350 mg (Yield 44%) of the title compound, (2-(aminomethyl)-5-fluorophenyl)(morpholino)-methanone hydrochloride as a white powder: ¹H NMR (300 MHz, DMSO-d6) δ ppm: 3.0-4.0 (8H, m), 3.78 (2H, t, J=5 Hz), 7.32 (1H, dd, J=8.8, 2.6 Hz), 7.35-7.44 (1H, t, J=8.5, 3 Hz), 7.75 (1H, dd, J=8.8, 5.5 Hz); LC/MS m/z 239 (M+H).

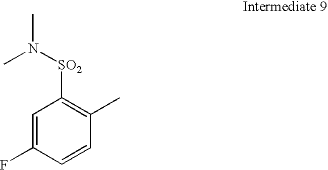

5-Fluoro-2,N,N-trimethyl-benzenesulfonamide. To a solution of 5-fluoro-2-methyl-benzenesulfonyl chloride (4.18 g, 20 mmol) in tetrahydrofuran (25 mL) was added, dropwise, a solution of dimethylamine in tetrahydrofuran (2M, 25 mL, 50 mmol) over 15 min. and the mixture stirred for 5 min. The insoluble materials were filtered and the filtrate concentrated. The residue was purified by column chromatography (SiO₂, 5% ether in CH₂Cl₂) to provide 4.3 g (Yield 90%) of the title compound as a clear oil: ¹H NMR (500 MHz, CDCl₃) δ ppm: 2.57 (3H, s) 2.82 (3H, s) 2.82 (3H, s) 7.12-7.18 (1H, m) 7.28 (1H, dd, J=8.2, 5.5 Hz) 7.59 (1H, dd, J=8.2, 2.1 Hz); LC/MC m/z 218 (M+H).

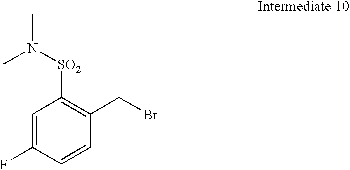

2-Bromomethyl-5-fluoro-N,N-dimethyl-benzenesulfonamide. Under nitrogen, a mixture of intermediate 45, 5-fluoro-2,N,N-trimethyl-benzenesulfonamide, (435 mg, 2.0 mmol) and N-bromosuccinimide (391 mg, 2.2 mmol) in CCl₄ (20 mL) was stirred at 80-90° C. for 5 min. To this mixture was added 2,2′-azobisisobutyronitrile (AIBN, 100 mg) and stirring continued at 80-90° C. for 30 min. After cooling, the insoluble precipitates were filtered and the filtrate concentrated and purified by column chromatography (SiO₂, CH₂Cl₂) to provide 440 mg (Yield 74%) of the title compound; ¹H NMR (500 MHz, CDCl₃) δ ppm: 2.87 (6H, s) 4.86 (2H, s) 7.28 (1H, dd, J=8.55, 2.75 Hz) 7.61-7.65 (2H, m); LC/MC m/z 296/298 (M+H).

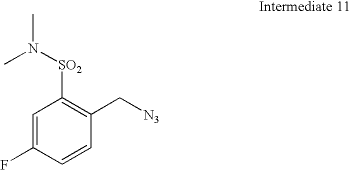

2-Azidomethyl-5-fluoro-N,N-dimethyl-benzenesulfonamide. A mixture of intermediate 46, 2-bromomethyl-5-fluoro-N,N-dimethyl-benzenesulfonamide, (880 mg, 2.97 mmol) and sodium azide (200 mg, 3 mmol) in dimethylformamide (4 mL) was stirred at 55-60° C. for 30 min after which the solvent was removed in vacuo. The residue was partitioned between CH₂Cl₂ and water, and the organic phase was washed with water, dried (Na₂SO₄), filtered and concentrated to provide 670 mg (Yield 87%) of the title compound as a yellow oil; ¹H NMR (500 MHz, CDCl₃) δ ppm: 2.84 (6H, s) 4.78 (2H, s) 7.29-7.34 (1H, m) 7.59-7.64 (2H, n).

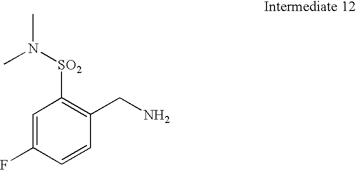

2-(Aminomethyl)-5-fluoro-N,N-dimethylbenzenesulfonamide. To a solution of intermediate 47, 2-azidomethyl-5-fluoro-N,N-dimethyl-benzenesulfonamide, (660 mg, 2.6 mmol) in tetrahydrofuran (10 mL) and water (2 mL) was added triphenylphosphine (740 mg, 2.8 mmol), and the mixture stirred under nitrogen for 1 hr. The tetrahydrofuran was evaporated in vacuo and a mixture of the residue and 6N HCl (3 mL) in MeOH (5 mL) was heated at 80° C. for 20 hrs. This was washed with CH₂Cl₂, and the aqueous phase basified with dilute NH₄OH and extracted with CH₂Cl₂. The organic extract was dried (Na₂SO₄), filtered and concentrated to provide 210 mg (0.91 mmol, Yield 35%) of the title compound; ¹H NMR (500 MHz, CDCl₃) δ ppm: 2.84 (6H, s) 4.10 (2H, s) 7.23-7.29 (1H, n) 7.53-7.60 (2H, m); LC/MS m/z 233 (M+H).

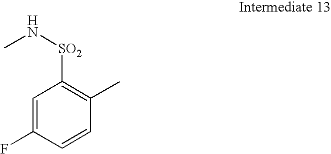

5-Fluoro-2,N-dimethyl-benzenesulfonamide. To a solution of 5-fluoro-2-methyl-benzenesulfonyl chloride (4.18 g, 20 mmol) in acetone (20 mL) was added a 40% aqueous solution of methylamine (4.5 mL, 60 mmol) under nitrogen and the mixture stirred for 5 min. Acetone was removed in vacuo and the aqueous residue extracted with CH₂Cl₂. The CH₂Cl₂ extract was dried (Na₂SO₄), filtered, concentrated and the residue purified by column chromatography (SiO₂, 10% ether in CH₂Cl₂) to provide 3.9 g (19.2 mmol, Yield 96%) of the title compound as a white solid; ¹H NMR (500 MHz, CDCl₃) δ ppm: 2.59 (3H, s), 2.67 (3H, d, J=5.5 Hz), 4.41 (1H, brs), 7.13-7.20 (1H, m), 7.29 (1H, dd, J=8.2, 5.5 Hz), 7.69 (1H, J=8.6, 2.1 Hz); LC/MS m/z 204 (M+H).

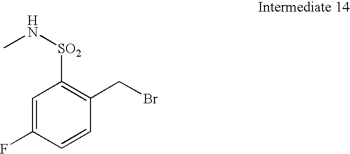

2-Bromomethyl-5-fluoro-N-methyl-benzenesulfonamide. The title compound can be prepared from intermediate 49, 5-fluoro-2,N-dimethyl-benzenesulfonamide, according to the method described for intermediate 46 and purified by column chromatography (SiO₂, 5% ether/CH₂Cl₂). ¹H NMR (500 MHz, CDCl₃) δ ppm: 2.64 (3H, d, J=5.19 Hz) 4.91 (1H, d, J=3.66 Hz) 4.98 (2H, s) 7.26-7.30 (1H, m) 7.54 (1H, dd, J=8.6, 5.2 Hz) 7.73 (1H, dd, J=8.4, 2.6 Hz); LC/MS m/z 282/284.

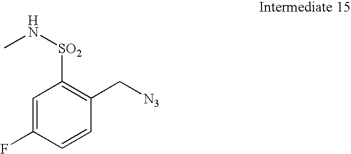

2-Azidomethyl-5-fluoro-N-benzenesulfonamide. The title compound can be prepared from intermediate 50, 2-bromomethyl-5-fluoro-N-methyl-benzenesulfonamide, according to the method described for intermediate 47 and purified by column chromatography (SiO₂, 5% ether-CH₂Cl₂). ¹H NMR (500 MHz, CDCl₃) δ ppm: 2.65 (3H, d, J=5.19 Hz) 4.81 (2H, s) 4.86 (1H, d, J=4.6 Hz) 7.27-7.33 (1H, m) 7.49 (1H, dd, J=8.2, 5.2 Hz) 7.76 (1H, dd, J=8.2, 2.8 Hz).

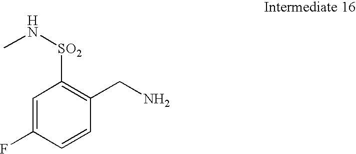

2-Azidomethyl-5-fluoro-N-benzenesulfonamide. The title compound solution of intermediate 51, 2-azidomethyl-5-fluoro-N-methyl-benzenesulfonamide, (560 mg, 2.3 mmol) in ethanol (10 mL) was added 6N HCl (1 mL) and 10% Pd—C (100 mg) and the mixture hydrogenated with 1 atm of H₂ for 14 hrs. The catalyst was removed by filtration through Celite and the filtrate concentrated in vacuo to provide 630 mg (Yield >100%) of the title compound. ¹H NMR (500 MHz, DMSO-D6) δ ppm: 4.36 (2H, d, J=5.2 Hz) 7.63-7.70 (2H, m) 7.77-7.83 (1H, m) 8.11 (1H, d, J=4.9 Hz) 8.41 (3H, s); LC/MS m/z 219 (M+H).

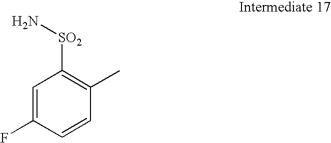

5-Fluoro-2-methyl-benzenesulfonamide. To a solution of 5-Fluoro-2-methyl-benzenesulfonamide chloride (4.18 g, 20 mmol) in acetone (20 mL) was added, dropwise, concentrated NH₄OH (3 mL) and the resulting mixture stirred for 5 min. Acetone was removed iv vacuo and the precipitates were filtered, washed thoroughly with water and dried in vacuo to provide 3.7 g (Yield 98%) of the title compound as a white solid; ¹H NMR (500 MHz, DMSO-D6) δ ppm: 2.55 (3H, s) 7.33-7.40 (1H, m) 7.40-7.46 (1H, m) 7.54 (2H, s) 7.59 (1H, dd, J=9.2, 2.7 Hz); LC/MS m/z 190 (M+H).

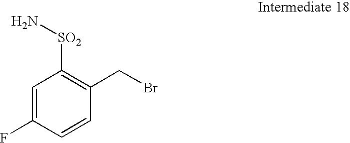

2-Bromomethyl-5-fluoro-benzenesulfonamide. The title compound can be prepared from intermediate 53, 5-fluoro-2-methyl-benzenesulfonamide, according to the method described for intermediate 46, and purified by column chromatography (SiO₂, 5% ether/CH₂Cl₂). ¹H NMR (500 MHz, CDCl₃) δ ppm: 5.01 (2H, s) 5.16 (2H, brs) 7.25-7.31 (1H, m) 7.53 (1H, dd, J=8.5, 5.2 Hz) 7.80 (1H, dd, J=8.5, 2.7 Hz). LC/MS m/z 268/270 (M+H).

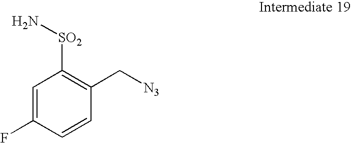

2-Azidomethyl-5-fluoro-N-methyl-benzenesulfonamide. The title compound can be prepared from intermediate 54, 2-bromomethyl-5-fluoro-benzenesulfonamide, according to the method described for the preparation of intermediate 47. ¹H NMR (300 MHz, CDCl₃) δ ppm: 4.82 (2H, s) 5.18 (2H, s) 7.27 (1H, m) 7.45 (H, dd, J=8.4, 5.5 Hz) 7.79 (1H, dd, J=8.4, 2.6 Hz). LC/MS m/z 253 (M+Na).

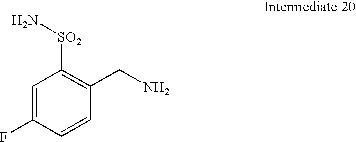

2-Azidomethyl-5-fluoro-N-methyl-benzenesulfonamide. The title compound can be prepared from intermediate 55, 2-azidomethyl-5-fluoro-N-methyl-benzenesulfonamide, according to the method described for the preparation of intermediate 48. ¹H NMR (500 MHz, DMSO-D6) δ ppm: 4.05 (2H, s) 5.05 (3H, br) 7.44 (1H, dt, J=8.5, 3 Hz) 7.58 (1H, dd, J=9.2, 2.7 Hz) 7.66 (1H, dd, J48.5, 5.5 Hz). LC/MS m/z 205 (M+H).

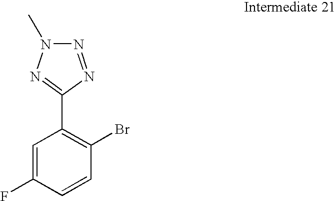

5-(2-Bromo-5-fluoro-phenyl)-2-methyl-2H-tetrazole. A mixture of 5-(2-bromo-5-fluoro-phenyl)-1H-tetrazole (1.0 g, 4.12 mmol), iodomethane (1.12 g, 10 mmol) and potassium carbonate (1.5 g) in dimethylformamide (5 mL) was stirred at room temperature for 16 hrs, then concentrated in vacuo. The residue was purified by column chromatography (SiO₂, CH₂Cl₂) to provide 650 mg (Yield 61%) of the title compound as a white powder. ¹H NMR (500 MHz, CDCl₃) δ ppm: 4.45 (3H, s) 7.03-7.11 (1H, m) 7.63 (1H, dd, J=8.9, 3.1 Hz) 7.69 (1H, dd, J=8.9, 5.5 Hz); ¹³C NMR (126 MHz, CDCl₃) δ ppm: 39.86, 116.28, 118.66, 118.76, 130.13, 135.73, 161.74, 163.53; LC/MS m/z 257/259.

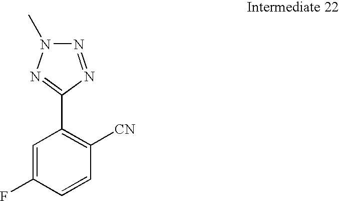

4-Fluoro-2-(2-methyl-2H-tetrazole-5-yl)-benzonitrile. A mixture of intermediate 57, 5-(2-bromo-5-fluoro-phenyl)-2-methyl-2H-tetrazole (650 mg, 2.53 mmol) and CuCN (224 mg, 2.5 mmol) in dimethylformamide (4 mL) was placed in a sealed tube and heated at 100-110° C. for 20 hrs. After cooling, the insoluble material was filtered, and the filtrate concentrated in vacuo. The residue was dissolved in CH₂Cl₂, washed with aq. 4N HCl and dil. NH₄OH, thien dried (MgSO₄), filtered, and concentrated. The residual solid was purified by column chromatography (SiO₂, CH₂Cl₂) to obtain 375 mg (Yield 73%) of the title compound as an off-white solid; ¹H NMR (500 MHz, CDCl₃) δ ppm: 4.48 (3H, s) 7.29 (1H, dd, J=7.6, 2.8 Hz) 7.85 (1H, dd, J=8.6, 5.2 Hz) 8.00 (1H, dd, J=9.0, 2.6 Hz); LC/MS m/z 204.

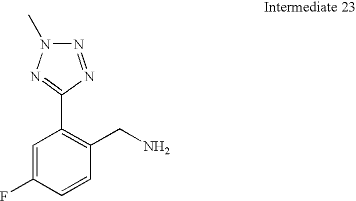

(4-Fluoro-2-(2H-tetrazol-5-yl)phenyl)methanamine hydrochloride. A solution of intermediate 58, 4-fluoro-2-(2-methyl-2H-tetrazol-5-yl)-benzonitrile, (330 mg, 1.62 mmol) in ethanol (15 mL) was mixed with 6N HCl (1 mL) and 10% Pd—C (200 mg) under nitrogen. The mixture was then stirred under hydrogen (1 atm) for 3 hrs. After removing the catalyst, the filtrate was concentrated in vacuo to provide 360 mg (Yield 91%) of the title compound as an off-white solid; ¹H NMR (500 MHz, DMSO-D6) δ ppm: 4.42 (2H, d, J=2.75 Hz) 4.49 (3H, s) 7.48-7.56 (1H, m) 7.78 (1H, dd, J=8.7, 5.7 Hz) 7.86 (1H, dd, J=9.8, 2.8 Hz) 8.45 (3H, s); LC/MS m/z 208.

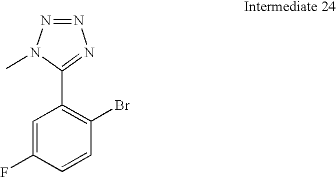

5-(2-Bromo-5-fluoro-phenyl)-1-methyl-2H-tetrazole. A mixture of 5-(2-bromo-5-fluoro-phenyl)-1H-tetrazole (1.0 g, 4.12 mmol), iodomethane (1.12 g, 10 mmol) and potassium carbonate (1.5 g) in dimethylformamide (5 mL) was stirred at room temperature for 16 hrs, then concentrated in vacuo. The residue was purified by column chromatography (SiO₂, CH₂Cl₂) to provide 350 mg (Yield 33%) of the title compound as white crystals. ¹H NMR (500 MHz, CDCl₃) δ ppm: 4.00 (3H, s) 7.18-7.25 (2H, m) 7.72 (1H, dd, J=8.4, 5.0 Hz); ¹³C NMR (126 MHz, CDCl₃) δ ppm: 34.59, 117.73, 119.58, 120.43, 127.57, 135.11, 153.43, 161.69. LC/MS m/z 257/259.

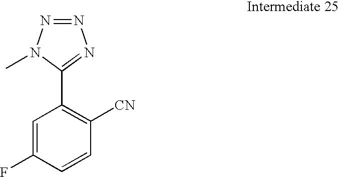

4-Fluoro-2-(1-methyl-2H-tetrazol-5-yl)-benzonitrile. ¹H NMR (300 MHz, CDCl₃) δ ppm: 4.13 (3H, s) 7.38-7.49 (2H, m) 7.86-7.97 (1H, m); LC/MS m/z 204 (M+H).

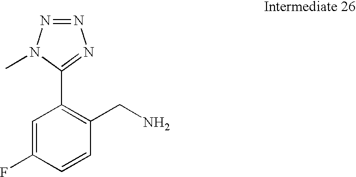

4-Fluoro-2-(1-methyl-2H-tetrazol-5-yl)phenyl)methanamine hydrochloride. ¹H NMR (500 MHz, DMSO-D6) δ ppm: 4.05 (2H, s) 4.09 (3H, s) 7.58-7.67 (1H, m) 7.77 (1H, dd, J=9.3, 2.6 Hz) 7.87 (1H, dd, J=8.7, 5.7 Hz) 8.38 (3H, s); LC/MS m/Z 208.

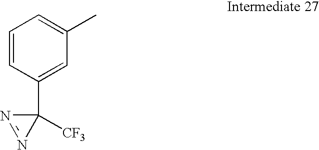

3-m-Tolyl-3-trifluoromethyl-3H-diazirine. To a cold stirring solution of 3-m-tolyl-3-trifluoromethyl-diaziridine (2.0 g, 10 mmol. prepared using the methods described in Doucet-Personeni C. et al., J. Med. Chem., 2001, 44, 3203 and Nassal, M. Liebigs Ann. Chem. 1983, 1510-1523 or in Stromgaard, K et al., J. Med. Chem., 2002, 45, 4038-46) in ethanol (20 mL) was added triethylamine (1.5 g, 15 mmol). To this mixture was added tert-butyl hypochlorite (3.25 g, 30 mmol), and the mixture stirred for 5 min. This mixture was poured into 10% aqueous sodium sulfite (100 mL), and extracted with ether. The ether extract was washed with brine, dried (MgSO₄), filtered and concentrated. The residue was purified by column chromatography (SiO₂, pentane) to provide 1.6 g (Yield 80%) of the title compound. ¹H NMR (300 MHz, CDCl₃) δ ppm: 2.33 (3H, s) 6.90-7.03 (2H, m) 7.15-7.31 (2H, m).

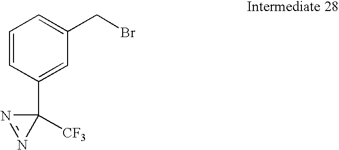

3-(3-Bromomethyl-phenyl)-3-trifluoromethyl-3H-trifluoromethyl-3H-diazirine. To a solution of intermediate 63, 3-m-tolyl-3-trifluoromethyl-3H-diazirine, (200 mg, 1 mmol) in CCl₄ (4 mL) was added N-bromosuccinimide (200 mg, 1.1 mmol, re-crystallized from water), and the stirred mixture heated at 85° C. To this was added AIBN (50 mg) and the mixture heated at reflux for an additional 2.5 hrs. After cooling, the mixture was purified by column chromatography (SiO₂, pentane) to provide 150 mg (Yield 54%) of the title compound as a clear oil. ¹H NMR (300 MHz, CDCl₃) δ ppm: 4.42 (2H, s) 7.10-7.17 (2H, m) 7.31-7.45 (2H, m).

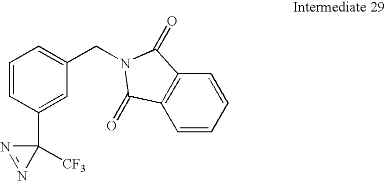

2-[3-(3-Trifluoromethyl)-diaziridin-3-yl)-benzyl]-isoindole-1,3-dione. A mixture of intermediate 64, 3-(3-bromomethyl-phenyl)-3-trifluoromethyl-3H-diazirine, (140 mg, 0.5 mmol) and potassium phthalimide (95 mg, 0.5 mmol) in dimethylformamide (1.5 mL) was stirred at room temperature for 3 hrs. Dimethylformamide was removed in vacuo. The residue was extracted with CH₂Cl₂, washed with water, then dried (Na₂SO₄), filtered, and concentrated. The resulting residue was purified by column chromatography (SiO₂, 1:1 CH₂Cl₂/pentane) to provide 140 mg (Yield 82%) of the title compound as a solid; ¹H NMR (300 MHz, CDCl₃) δ ppm: 4.80 (2H, s) 7.09-7.21 (2H, m) 7.32 (1H, t, J=7.9 Hz) 7.41-7.49 (2H, m) 7.66-7.71 (2H, m) 7.81-7.85 (2H, m); LC/MS m/z 346 (M+H).

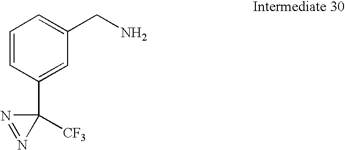

(3-(3-(Trifluoromethyl)diaziridin-3-yl)phenyl)methanamine. A stirred solution of intermediate 65, 2-[3-(3-trifluoromethyl-diaziridin-3-yl)-benzyl]-isoindole-1,3-dione, (150 mg, 0.43 mmol) in ethanol (2 mL) was treated with hydrazine hydrate (0.4 mL) at room temperature and the solution stirred for 3.5 hrs. After removing ethanol in vacuo, the residue was partitioned between CH₂Cl₂ and water. The aqueous phase was acidified with dilute HCl, and washed with CH₂Cl₂. The aqueous phase was basified with dilute NaOH, and extracted with CH₁₂Cl₂. The organic extract was dried (MgSO₄), filtered, and concentrated to obtain 50 mg (Yield 54%) of (3-(3-(trifluoromethyl)diaziridin-3-yl)phenyl)methanamine and (3-(3-(trifluoromethyl)-3H-diazirin-3-yl)phenyl)methanamine as a 1:1 mixture; ¹H NMR (300 MHz, CDCl₃) δ ppm: 3.85 (2H, s) 3.88 (2H, s) 7.08 (2H, s) 7.31-7.40 (4H, m) 7.43-7.50 (1H, m, J=6.2 Hz) 7.54 (1H, s); LC/MS m/z 216 (M+H for diazirine) and 218 (M+H for diaziridine).

### Intermediates 31-32

To a solution of 2,4-difluorobenzonitrile (10 g, 72 mmol) dissolved in tetrahydrofuran (20 mL), and dimethylformamide (40 mL) was added 1,2,4-triazole sodium salt (6.3 g, 70 mmol) and the mixture stirred at 90° C. for 3 h after which the mixture was filtered and the solvent removed. The resulting residue was adsorbed onto silica gel and intermediates 67 and 68 separated by flash chromatography, eluting with 0% to 30% ethyl acetate/hexanes.

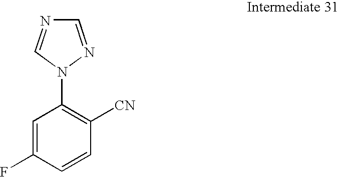

4-Fluoro-2-(1H-1,2,4-triazol-1-yl)benzonitrile. Colorless needles (2.46 g, 18% yield) ¹H NMR (500 MHz, CDCl₃) δ: 8.89 (1H, s), 8.19 (1H, s), 7.85 (1H, dd, J=8.7, 5.6 Hz), 7.60 (1H, dd, J=8.8, 2.4 Hz), 7.28-7.24 (1H, m). LCMS (M+H) calcd for C₉H₆N₄F: 189.05. found: 189.13.

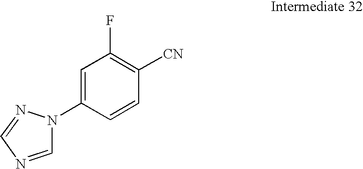

4-(1H-1,2,4-Triazol-1-yl)-2-fluorobenzonitrile. White solid (0.746 g, 6% yield) ¹H NMR (500 MHz, CDCl₃) δ: 8.66 (1H, s), 8.15 (1H, s), 7.79 (1H, dd, J=8.5, 6.7 Hz), 7.69 (1H, dd, J=9.5, 1.8 Hz), 7.65-7.63 (1H, m). LCMS (M+H) calcd for C₉H₆N₄F: 189.05. found: 189.13.

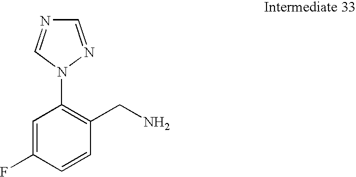

(4-Fluoro-2-(1H-1,2,4-triazol-1-yl)phenyl)methanamine hydrochloride). Intermediate 67, 4-fluoro-2-(1H-1,2,4-triazol-1-yl)benzonitrile, (2.46 g, 13.13 mmol) was dissolved in hot ethanol (150 mL). To this was added 1N HCl (15 mL) followed by 10% Pd—C (200 mg). The mixture was treated with H₂ at 55 psi for 4 h in a Parr shaker then filtered over Celite and the solvent removed under reduced pressure. The resulting residue was partitioned between ethyl acetate and water. The aqueous phase was separated and lyophilized to afford the title compound as a white powder (2.96 g, 99% yield). ¹H NMR (500 MHz, CD₃OD) δ ppm: 9.51 (1H, s), 8.63 (1H, s), 7.85 (1H, dd, J=8.5, 5.8 Hz), 7.68 (1H, dd, J=8.8, 2.4 Hz), 7.49 (1H, td, J=8.3, 2.4 Hz), 4.20 (2H, s). LCMS (M+H) calcd for C₉H₁₀N₄F: 193.08. found: 193.16.

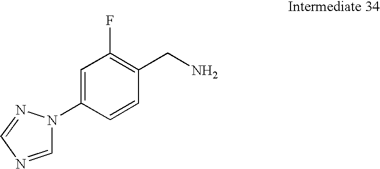

(2-Fluoro-4-(1H-1,2,4-triazol-1-yl)phenyl)methanamine hydrochloride. The title compound can be prepared from intermediate 68 according to the method described for the synthesis of intermediate 69. White powder (79% yield). ¹H NMR (500 MHz, CD₃OD) δ ppm: 9.25 (1H, s), 8.46 (1H, s), 7.80 (1H, dd, J=8.6, 5.8 Hz), 7.64 (1H, dd, J=8.8, 2.4 Hz), 7.44 (1H, td, J=8.3, 2.6 Hz), 4.17 (2H, s). LCMS (M+H) calcd for C₉H₁₀N₄F: 193.08. found: 193.16.

### Intermediates 35-38

Intermediates 71-74 were prepared using the procedure described for the synthesis of intermediate 67-70.

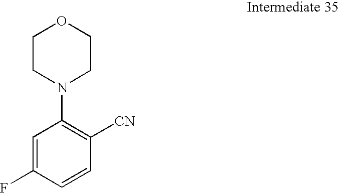

4-Fluoro-2-morpholinobenzonitrile ¹H NMR (500 MHz, CDCl₃) δ ppm: 7.55 (1H, dd, J=8.5, 6.4 Hz), 6.71 (1H, td, J=8.1, 2.3 Hz), 6.67 (1H, dd, J=11.0, 2.4 Hz), 3.88 (4H, t, J=4.6 Hz), 3.22 (4H, t, J=4.6 Hz). LCMS (M+H) calcd for C₁₁H₁₂N₂OF: 207-09. found: 207.19.

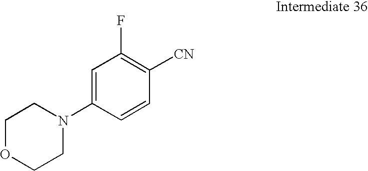

4-Morpholino-2-fluorobenzonitrile. ¹H NMR (500 MHz, CDCl₃) δ ppm: 7.42 (1H, dd, J=8.8, 7.6 Hz), 6.63 (1H, dd, J=8.8, 2.4 Hz), 6.56 (1H, dd, J=12.8, 2.4 Hz), 3.84 (4H, t, J=4.9 Hz), 3.28 (4H, t, J=4.9 Hz). LCMS (M+H) calcd for C₁₁H₁₂N₂OF: 207.09. found: 207.19.

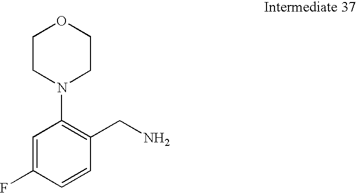

(4-Fluoro-2-morpholinophenyl)methanamine hydrochloride. ¹H NMR (500 MHz, CDCl₃) δ ppm: 7.54 (1H, t, J=7.3 Hz), 7.20 (1H, dd, J=10.5, 2.0 Hz), 7.05-7.02 (1H, m), 4.28 (2H, s), 3.93 (4H, bs), 3.03 (4H, bs). LCMS (M+H) calcd for C₁₁H₁₆N₂OF: 211.12. found: 211.23.

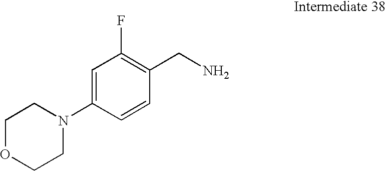

(2-Fluoro-4-morpholinophenyl)methanamine hydrochloride. ¹H NMR (500 MHz, CD₃OD) δ ppm: 7.73 (1H, t, J=8.2 Hz), 7.62 (1H, d, J=7.6 Hz), 7.58 (1H, d, J=8.2 Hz), 4.26 (2H, s), 4.11 (4H, t, J=4.4 Hz), 3.65 (4H, t, J=4.4 Hz). LCMS (M+H) calcd for C₁₁H₁₆N₂OF: 211.12. found: 211.23.

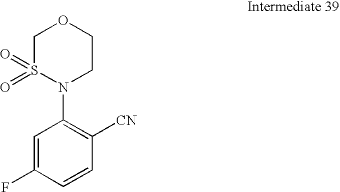

4-Fluoro-2-(1,1-dioxo-1λ⁶-[1,2]thiazinan-2-yl)benzonitrile. To a mixture of 2,4-difluorobenzonitrile (10.0 g, 72 mmol) and 1,1-dioxo-1λ6-[1,2]thiazin-2-ane (8.84 g, 65.4 mmol) in 1:1 tetrahydrofuran/dimethylformamide (40 mL) was added potassium carbonate (9.0 g, 65.4 mmol). The mixture was stirred at 90° C. for 18 h then filtered and concentrated. The residue was purified by flash chromatography (SiO₂) eluting with 10%-50% ethyl acetate/hexanes followed by recrystallization from hot ethyl acetate/hexane to give the title compound as white needles (0.537 g, 3% yield). ¹H NMR (500 MHz, CD₃OD) δ ppm: 7.70 (1H, dd, J=8.8, 5.8 Hz), 7.30 (1H, dd, J=8.8, 2.4 Hz), 7.15-7.12 (1H, m), 3.27 (2H, t, J=5.3 Hz), 3.33 (2H, t, J=6.1 Hz), 2.40-2.35 (2H, m), 2.05-2.01 (2H, m). LCMS (M+H) calcd for C₁₁H₁₆N₂OF: 255.06. found: 255.19.

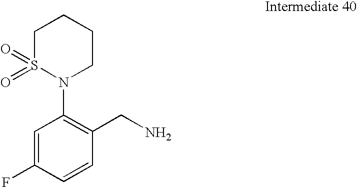

(4-Fluoro-2-(1,1-dioxo-1λ⁶-[1,2]thiazinan-2-yl)phenyl)methanamine hydrochloride. Intermediate 75, 4-fluoro-2-(1,1-dioxo-1λ⁶-[1,2]thiazinan-2-yl)benzonitrile (1.37 g, 5.4 mmol) was dissolved in ethanol (120 mL). To this was added 1N HCl (20 mL) and a catalytic amount of 10% Pd—C. The mixture was shaken under hydrogen at 55 psi for 4 h then filtered through Celite and concentrated to give the title compound as white solid (1.58 g, 100% yield). ¹H-NMR (300 MHz, CD₃OD) δ ppm: 7.61 (1H, dd, J=8.4, 6.2 Hz), 7.38 (1H, dd, J=9.3, 2.7 Hz), 7.28 (1H, td, J=8.2, 2.7 Hz), 7.26 (2H, dd, J=21.4, 13.7 Hz), 3.93-3.84 (1H, m), 3.50-3.41 (3H, m), 2.40-2.31 (2H, m), 2.04-1.96 (2H, m). LCMS [M+H]⁺ calcd for C₁₁H₁₆N₂O₆FS: 259.087. found: 259.24.

### Intermediates 41-42

To a solution of 1H-1,2,3-triazole (3.5 g, 50.7 mmol) in tetrahydrofuran (10 mL) and dimethylformamide (20 mL) was added, portionwise, NaH (1.3 g, 51 mmol, 95%). The mixture was stirred at room temp for 30 min. 2,4-Difluorobenzonitrile (7.6 g, 55 mmol) was added and the mixture stirred at 85° C. for 3 h. The white mixture was concentrated and purified by flash chromatography eluting with 0% to 10% ethyl acetate/hexanes to give intermediates 77 and 78.

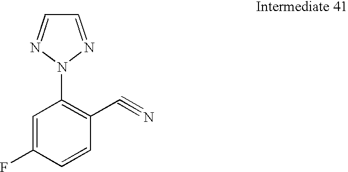

4-Fluoro-2-1,2,3-triazol-2-yl-benzonitrile. White needles (0.34 g, 3% yield). 1H-NMR (300 MHz, CDCl₃) δ ppm: 7.92 (2H, s), 7.88-7.79 (2H, m), 7.19-7.12 (1H, m). LCMS [M+H]+ calcd for C₉H₆N₄F; 189.05. found: 189.12.

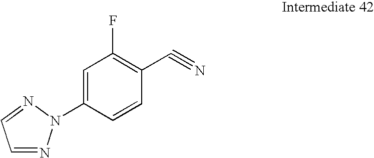

2-Fluoro-4-1,2,3-triazol-2-yl-benzonitrile. White solid (0.097 g, 1% yield). ¹H-NMR (300 MHz, CDCl₃) δ ppm: 8.03-7.95 (2H, m), 7.86 (2H, s), 7.74-7.69 (1H, m).

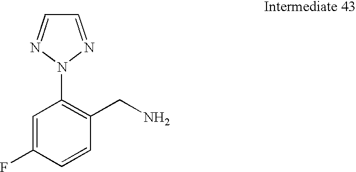

4-Fluoro-2-1,2,3-triazol-2-yl-benzylamine hydrochloride. Intermediate 77, 4-fluoro-2-1,2,3-triazol-2-yl-benzonitrile, (0.34 g, 1.8 mmol) was dissolved in ethanol (50 mL). 1N HCl (10 ml) was added along with a catalytic amount of 10%-Pd—C. The mixture was shaken under H₂ at 55 psi for 4 h after which it was filtered through Celite and concentrated to give the title compound as the corresponding HCl salt. Yellow solid (0.402 g, 98% yield). ¹H-NMR (500 MHz, CD₃OD) δ ppm: 8.13 (2H, s), 7.87 (1H, dd, J=4.9, 2.6 Hz), 7.73 (1H, dd, J=4.9, 2.6 Hz), 7.34 (1H, td, J=8.2, 2.7 Hz), 4.35 (2H, s). LCMS [M+H]⁺ calcd for C₉H₁₀N₄F: 193.08. found: 193.16.

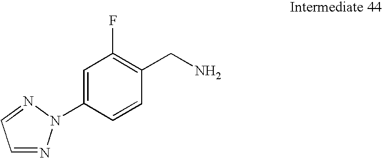

(2-Fluoro-4-(2H-1,2,3-triazol-2-yl)phenyl)methanamine: The title compound can be prepared from intermediate 78, 2-fluoro-4-1,2,3-triazol-2-yl-benzonitrile, according to the procedure provide for intermediate 79. ¹H-NMR (300 MHz, CD₃OD) δ ppm: 8.05-7.96 (2H,m), 8.00 (2H, s), 7.68 (1H, t, J=8.2 Hz), 4.26 (2H, s). LCMS [M+H]⁺ calcd for C₉H₁₀N₄F: 193.08. found: 193.14.

### Intermediates 45-48

A solution of 2,4-difluorobenzonitrile (7.07 g, 50.8 mmol) and 3-methyl-1H-1,2,4-triazole (4.22 g, 50.8 mmol) in N,N-dimethylformamide (45 ml) was treated with powdered anhydrous potassium carbonate (10 g) and the resulting mixture stirred at 22° C. for 18 h. The solid was then filtered and the filtrate concentrated in vacuo. The residue was diluted with ethyl acetate, washed with water and brine, then dried over anhydrous magnesium sulfate and concentrated. The resulting mixture was purified by a combination of chromatography on silica gel (elution gradient of ethyl acetate in hexane) and reversed phase silica gel to yield intermediates 81-84.

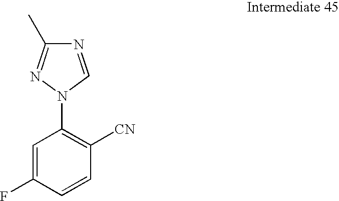

4Fluoro-2-(3-methyl-1H-1,2,4-triazol-1-yl)benzonitrile. White crystals (ethyl acetate-hexane); mp 117-118° C. ¹HNMR 400 MHz (CDCl₃) δ ppm: 2.54 (3H, s, CH₃), 7.24 (1H, m, CH), 7.62 (1H, dd, J=2.5 Hz and J=9.1 Hz, CH), 7.84 (1H, dd, J=5.6 Hz and J=8.6 Hz, CH), 8.82 (1H, s, CH). Anal. Calcd for C₁₀H₇FN₄: C, 59.40; H, 3.49; N, 27.71. Found: C, 59.25; H, 3.32; N, 27.81.

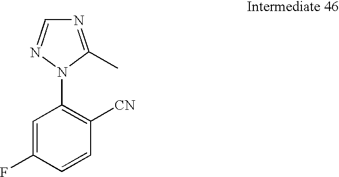

4-Fluoro-2-(5-methyl-1H-1,2,4-triazol-1-yl)benzonitrile. White crystals (ethyl acetate-hexane); mp 120-121° C. ¹HNMR 400 MHz (CDCl₃) δ ppm: 2.56 (3H, s, CH₃), 7.30 (1H, dd, J=2.5 Hz and J=8.1 Hz, CH), 7.39 (1H, m, CH), 7.91 (1H, dd, J=5.5 Hz and J=8.6 Hz, CH), 8.06 (1H, s, CH). Anal. Calcd for C₁₀H₇FN₄: C, 59.40; H, 3.49; N, 27.71. Found: C, 59.35; H, 3.70; N, 27.77.

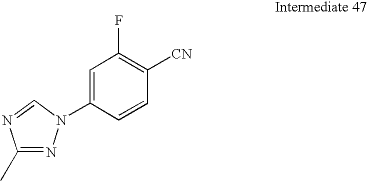

2-Fluoro-4-(3-methyl-1H-1,2,4-triazol-1-yl)benzonitrile. White crystals (ethyl acetate-hexane); mp 133-134° C. ¹HNMR 400 MHz (CDCl₃) δ ppm: 2.52 (3H, s, CH₃), 7.61 (1H, dd, J=2 Hz and J=9.1 Hz, CH), 7.67 (1H, dd, J=2 Hz and J=9.6 Hz, CH), 7.79 (1H, dd, J=6.5 Hz and J=8.6 Hz, CH), 8.56 (1H, s, CH). Anal. Calcd for C₁₀H₇FN₄: C, 59.40; H, 3.49; N, 27.71. Found: C, 59.42; H, 3.24; N, 28.41.

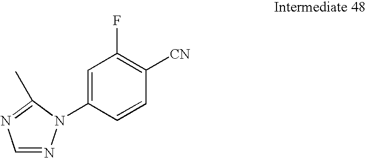

2-Fluoro-(5-methyl-1H-1,2,4-triazol-1-yl)benzonitrile. White crystals (ethyl acetate-hexane); mp 89-90° C., ¹HNMR 400 MHz (CDCl₃) δ ppm: 2.69 (3H, s, CH₃), 7.49-7.55 (2H, m, 2×CH), 7.83 (1H, dd, J=6.8 Hz and J=8.8 Hz, CH), 8.00 (1H, s, CH). Anal. Calcd for C₁₀H₇FN₄: C, 59.40; H, 3.49; N, 27.71. Found: C, 59.17; H, 3.22; N, 28.01.

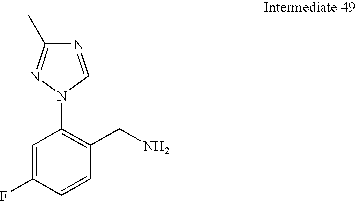

(4-Fluoro-2-(3-methyl-1H-1,2,4-triazol-1-yl)phenyl)methanamine hydrochloride salt. Hydrogenation of intermediate 81, 4-fluoro-2-(3-methyl-1H-1,2,4-triazol-1-yl)benzonitrile, (0.680 g, 3.36 mmol) gave 0.720 g (88% yield) of the title hydrochloride salt as a white solid. ¹HNMR 400 MHz (DMSO-d₆) δ ppm: 2.40 (3H, s, CH₃), 4.02 (2H, m, NCH₂), 7.50 (1H, nm, CH), 7.62 (1H, dd, J=2.8 Hz and J=9.3 Hz, CH), 7.84 (1H, dd, J=6.1 Hz and J=9.1 Hz, CH), 9.00 (1H, s, CH). HRMS (ESI⁺) calculated for C₁₀H₁₂FN₄ [M+H⁺]: 207.1046. found: 207.1047.

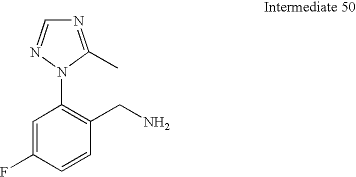

(4-Fluoro-2-(5-methyl-1H-1,2,4-triazol-1-yl)phenyl)methanamine hydrochloride salt. Hydrogenation of intermediate 82, 4-fluoro-2-(5-methyl-1H-1,2,4-triazol-1-yl)benzonitrile, (0.244 g, 1.20 mmol) gave 0.290 g (100% yield) of the title hydrochloride salt as a white solid. ¹HNMR 400 MHz (DMSO-d₆) δ ppm: 2.42 (3H, s, CH₃), 3.78 (2H, m, NCH₂), 7.58 (1H, m, CH), 7.67 (1H, dd, J=2.8 Hz and J=9.3 Hz, CH), 7.90 (1H, dd, J=6.0 Hz and J=8.6 Hz, CH), 8.22 (1H, s, CH). HRMS (ESI⁺) calculated for C₁₀H₁₂FN₄ [M+H⁺]: 207.1046. found: 207.1041.

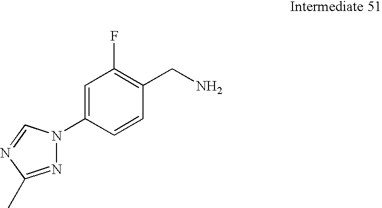

(2-Fluoro-4-(3-methyl-1H-1,2,4-triazol-1-yl)phenyl)methanamine hydrochloride salt. Hydrogenation of intermediate 83, 2-fluoro-4-(3-methyl-1H-1,2,4-triazol-1-yl)benzonitrile, (0.220 g, 1.09 mmol) gave 0.260 g (98% yield) of the title hydrochloride salt as a white solid. ¹HNMR 400 MHz (DMSO-d₆) δ ppm: 2.38 (3H, s, CH₃), 4.09 (2H, m, NCH₂), 7.75-7.8 (2H, m, 2×CH), 7.83 (1H, dd, J=2 Hz and J=9 Hz, CH), 9.29 (1H, s, CH). MS (ESI⁺) m/e 207 [M+H⁺].

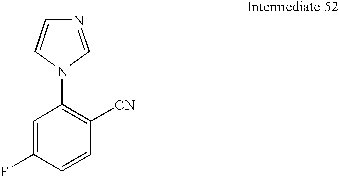

4-Fluoro-2-imidazol-1-yl-benzonitrile. To a solution of imidazole (4.45 g, 65.4 mmol) in tetrahydrofuran (30 mL) and dimethylformamide (10 mL) was added potassium carbonate (9.95 g, 72 mmol) and the mixture was stirred for 30 min at room temp. To this was added 2,4-difluorobenzonitrile (10.0 g, 72 mmol) and the mixture stirred at 90° C. for 3 h then at room temp for 2 days. The mixture was filtered and concentrated and the residue was purified by flash chromatography (SiO₂) eluting with 20% to 70% ethyl acetate/hexane to give the title compound as white needles (1.1 g, 9% yield). ¹H-NMR (500 MHz, CDCl₃) δ ppm: 7.94 (1H, s), 7.84 (1H, dd, J=8.7, 5.6 Hz), 7.37 (1H, t, J=8.7, 5.6 Hz), 7.37 (1H, t, J=1.4 Hz), 7.29 (1H, t, J=1.1 Hz), 7.27-7.21 (2H, m). LCMS [M+H]⁺ calcd for C₁₀H₇N₃F: 188.058. found: 188.12.

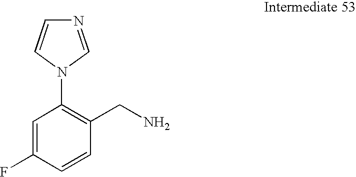

(4-Fluoro-2-(1H-imidazo-1-yl)phenyl)methanamine)hydrochloride. The title compound can be prepared from intermediate 88, 4-fluoro-2-imidazol-1-yl-benzonitrile, according to the method provided for intermediate 79. Yellow solid, ¹H-NMR (500 MHz, CD₃OD) δ ppm: 9.39 (1H, s), 7.98 (1H, d, J=1.5 Hz), 7.92-7.89 (2H, m), 7.63-7.59 (2H, m), 4.11 (2H, s). LCMS [M+H]⁺ calcd for C₁₀H₁₁N₃F: 192.09. found: 192.15.

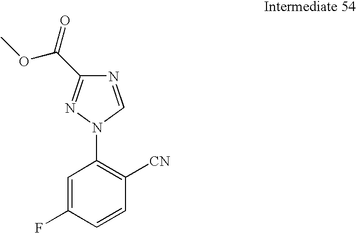

1-(2-Cyano-5-fluoro-phenyl)-1H-1,2,4-triazole-3-carboxylic acid methyl ester. To a solution of methyl 1H-1,2,4-triazole-3-carboxylate (27 g, 215 mmol) in dimethylformamide (170 mL) was added sodium hydride (5.53 g, 95%, 217 mmol) and the mixture was stirred for 30 min. Added to this was 2,4-difluorobenzylnitrile (30 g, 217 mmol) and the resulting mixture stirred at room temp for 60 h. The mixture was diluted with water and filtered to remove solids. The solution was extracted with ethyl acetate and the organic phase was washed with water (3×'s) and brine, then dried (Na₂SO₄) and concentrated. The resulting residue was purified by flash chromatography (SiO₂) eluting with 30% tetrahydrofuran/20% CH₂Cl₂/50% hexane to give the title compound as white needles (5.34 g, 10% yield). ¹H-NMR (300 MHz, CDCl₃) δ ppm: 8.92 (1H, s), 7.85 (1H, dd, J=8.8, 5.5 Hz), 7.67 (1H, dd, J=8.8, 2.6 Hz), 7.34-7.27 (1H, m), 40.3 (3H, s). LCMS [M+H]⁺ calcd for C₁₁H₈N₄FO₂: 247.06. found: 247.11.

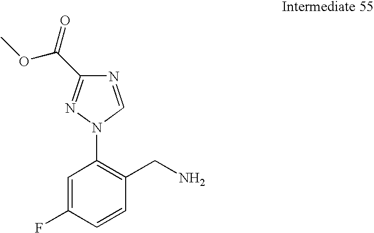

Methyl 1-(2-(aminomethyl)-5-fluorophenyl)-1H-1,2,4-triazole-3-carboxylate. The title compound can be prepared from intermediate 90, 1-(2-cyano-5-fluoro-phenyl)-1H-1,2,4-triazole-3-carboxylic acid methyl ester ¹H-NMR (300 MHz, CD₃OD) δ ppm: 9.15 (1H, s), 7.80 (1H, dd, J=8.8, 5.9 Hz), 7.71 (1H, dd, J=8.8, 2.6 Hz), 7.46 (1H, dd, J=8.2, 2.6 Hz), 4.19 (2H, s), 4.03 (3H, s). LCMS [M+H]⁺ calcd for C₁₁H₁₂N₄O₂: 251.09. found: 251.17.

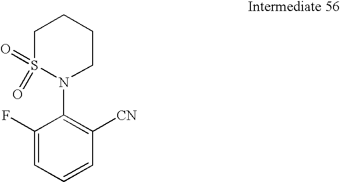

3-Fluoro-2-(1,1-dioxo-1λ6-[1,2]thiazinan-2-yl)benzonitrile. To a solution of 1,1-dioxo-1λ6-[1,2]thiazin-2-ane (1.90 g, 14.4 mmol) dissolved in tetrahydrofuran (8 mL) and dimethylformamide (2 mL) was added sodium hydride (0.36 g, 95%, 14.4 mmol) and the mixture stirred for 20 min. To this was added 2,3-difluorobenzonitrile (2.0 g, 14.4 mmol) and the mixture stirred at 90° C. for 2 h. The Mixture was partitioned between ethyl acetate and water. The organic phase was washed with water and brine then concentrated. The solid residue was triturated with 1:1 ethyl acetate/hexane to give the title compound as a pale brown solid (0.47 g, 13% yield). ¹H-NMR (500 MHz, CDCl₃) δ ppm: 7.47-7.45 (1H, m), 7.32-7.36 (2H, m), 4.08-4.02 (1H, m), 3.57 (1H, td, J=13.0, 3.7 Hz), 3.40-3.34 (1H, m), 3.32-3.27 (1H, m), 2.44-2.32 (2HF, m), 2.04-1.97 (2H, m), 1.90-1.84 (1H, m). LCMS [M+H]⁺ calcd for C₁₁H₁₂N₂FO₂S: 255.28. found: 255.13.

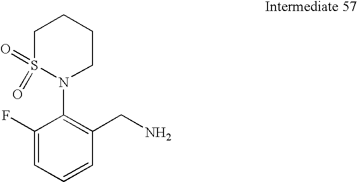

3-Fluoro-2-(1,1-dioxo-1λ6-[1,2]thiazinan-2-yl)benzonitrile hydrochloride. The title compound can be prepared from intermediate 92, 3-fluoro-2-(1,1-dioxo-1λ6-[1,2]thiazinan-2-yl)benzonitrile according to the procedure provided for intermediate 79. White solid, ¹H-NMR (500 MHz, CD₃OD) δ ppm: 7.56-7.52 (1H, m), 7.40-7.34 (2H, m), 4.31 (2H, s), 3.98-3.93 (1H, m), 3.68-3.64 (1H, m), 3.42-3.39 (2H, m), 2.42-2.37 (2H, m), 2.03-1.92 (2H, m). LCMS [M+H]⁺ calcd for C₁₁H₁₆N₂O₂FS: 259.09. found: 259.18.

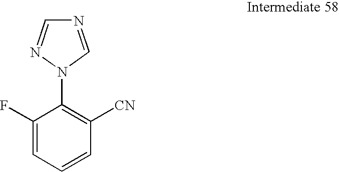

3-Fluoro-2-1,2,4-triazol-1-yl-benzonitrile. A mixture of 2,3-difluorobenzylnitrile (2.27 g, 16.3 mmol) and triazole sodium salt (1.33 g, 14.8 mmol) in tetrahydrofuran (5 mL) and dimethylformamide (10 mL) was stirred at 85° C. for 4 h. After concentration, the residue was purified by flash chromatography (SiO₂) eluting with 25%-50% ethyl acetate/hexane. The isolated product was recrystallized from hot ethyl acetate/hexane to give the title compound as white needles (1.51 g, 54% yield). ¹H-NMR (500 MHz, CDCl₃) δ ppm: 8.50 (1H, d, J=2.4 Hz), 8.25 (1H, s), 7.69-7.67 (1H, m), 7.60-7.57 (2H, m). LCMS [M+H]⁺ calcd for C₉H₆N₄F: 189.16. found: 189.14.

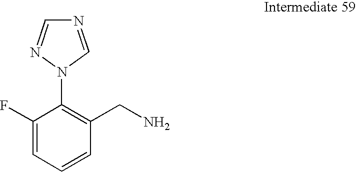

(3-Fluoro-2-(1H-1,2,4-triazol-1-yl)phenyl)methanamine. The title compound can be prepared from intermediate 94, 3-fluoro-2-1,2,4-triazol-1-yl-benzonitrile. ¹H-NMR (500 MHz, CD₃OD) δ ppm: 9.61 (1H, d, J=2.9 Hz), 8.79 (1H, s), 7.82-7.74 (1H, m), 7.67-7.57 (2H, m), 4.14-4.13 (2H, m). LCMS [M+H]⁺ calcd for C₉H₁₀N₄F: 193.08. found: 193.16.

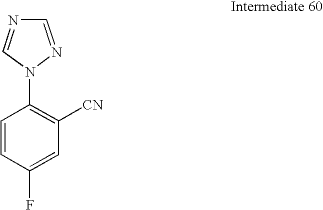

5-Fluoro-2-(1H-1,2,4-triazol-1-yl)benzonitrile. A suspension of 2,5-difluorobenzonitrile (4.5 g, 32.35 mmol) and 1,2,4-triazole sodium salt (3.6 g, 40 mmol) in dimethylformamide (40 mL) was heated at 80° C. for 15 h. The reaction mixture was then cooled, diluted with CH₂Cl₂ (200 mL), washed with water (3×30 mL) and brine (30 mL), then dried (Na₂SO₄), filtered and concentrated to give a white solid which was purified by flash column chromatography (SiO₂) using 1:1 to 3:1 ethyl acetate/Hexanes to afford the title compound (2.98 g, 49% yield) as a white powder. ¹H NMR (500 MHz, CDCl₃) δ: 8.70 (1H, s), 8.18 (1H, s), 7.76 (1H, dd, J=9.0, 4.8 Hz), 7.55 (1H, dd, J=7.3, 2.8 Hz), 7.51-7.47 (1H, m). LCMS (M+H) calcd for C₉H₆FN₄: 189.17. found: 189.10.

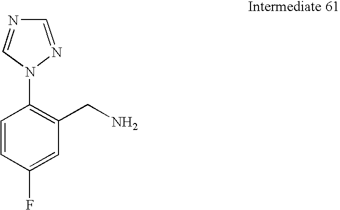

(5-Fluoro-2-(1H-1,2,4-triazol-1-yl)phenyl)methanamine hydrochloride. A solution of intermediate 96, 5-fluoro-2-(1H-1,2,4-triazol-1-yl)benzonitrile (2.94 g, 15.59 mmol) in ethanol (100 mL) and 1N HCl (50 mL) was degassed by bubbling N₂. Then, 10% Pd/C was added, the flask evacuated and vented to H₂ three times and left on a Parr shaker under a H₂ atmosphere (40 psi). After 6 h, the reaction mixture was filtered, concentrated and the aqueous solution lyophilized to afford the title compound (4.07 g, 98%) as a white powder. LCMS (M+H) calcd for C₉H₁₀FN₄: 193.09. found: 193.15.

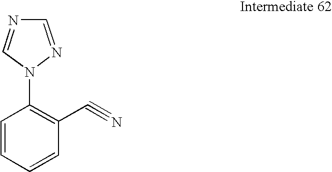

2-(1H-1,2,4-Triazol-1-yl)benzonitrile. A suspension of 2-fluorobenzylnitrile (3.0 g, 25 mmol) and 1,2,4-triazole sodium salt (2.4 g, 27 mmol) were stirred in tetrahydrofuran (7 mL) and dimethylformamide (14 mL) at 95° C. for 18 h. After cooling and concentrating, the product was crystallized from hot CH₂Cl₂/hexane (1:1) to give the title compound as a white solid (4.25 g, 100% yield). ¹H-NMR (300 MHz, CDCl₃) δ ppm: 8.74 (1H, s), 8.16 (1H, s), 7.82 (1H, dd, J=4.9, 1.3 Hz), 7.77-7.25 (2H, m), 7.57-7.51 (1H, m). LCMS [M+H]⁺ calcd for C₉H₇N₄: 171.06. found: 171.12.

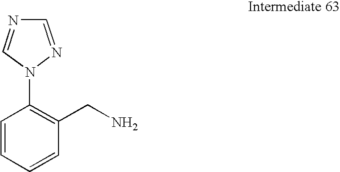

(2-(1H-1,2,4-Triazol-1-yl)phenyl)methanamine hydrochloride. Intermediate 98, 2-(1H-1,2,4-triazol-1-yl)benzonitrile (4.25 g, 25 mmol) was dissolved in ethanol (50 mL) and 1N HCl (25 mL). 10% Pd—C (1 g) was added and the mixture shaken under H₂ for 2 h at 50 psi. After filtration through Celite and concentration, the residue was triturated with diethyl ether and the title compound was collected as a white solid. (3.94 g, 75% yield). ¹H-NMR (300 MHz, CD₃OD) δ ppm: 9.01 (1H, s), 8.32 (1H, s), 7.78-7.64 (4H, m), 4.15 (2H, s). LCMS [M+H]⁺ calcd for C₉H₁₀N₄: 175.09. found: 175.17.

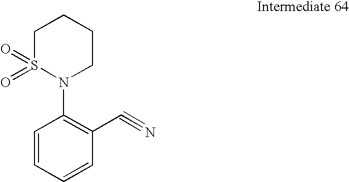

2-(1,1-Dioxo-1λ⁶-[1,2]thiazinan-2-yl)benzonitrile. Added to a solution of 1,1-dioxo[1,2]thiazinane (3.37 g, 25 mmol) in dimethylformamide (35 mL) was sodium hydride (0.675 g, 25 mmol, 95%) and the mixture stirred at room temperature for 15 min. 2-Fluorobenzonitrile (3.37 mL, 31.3 mmol) was added and the mixture stirred at 80° C. for 18 h. The mixture was cooled, diluted with water and extracted with ethyl acetate. The organic phase was washed with water and brine, then dried (Na₂SO₄) and concentrated. The residue was purified by flash chromatography (SiO₂) eluting with 10%-100% ethyl acetate/hexane. The isolated solid was recrystallized from hot ethyl acetate/hexane (2:1) to give the title compound as white crystals (4.15 g, 70% yield). ¹H-NMR (300 MHz, CDCl₃) δ ppm: 7.70 (1H, dd, J=7.7, 1.1 Hz), 7.64-7.53 (2H, m), 7.41 (1H, td, J=7.3, 1.6 Hz), 3.72 (2H, t, J=5.5 Hz), 3.32 (2H, t, J=6.0 Hz), 2.40-2.32 (2H, m), 2.05-1.97 (2H, m). LCMS [M+H]⁺ calcd for C₁₁H₁₂N₂O₂S: 237.06. found: 237.10.

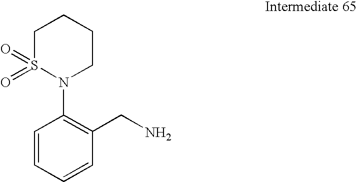

2-(1,1-Dioxo-1λ⁶-[1,2]thiazinan-2-yl)benzylamine hydrochloride. Intermediate 100, 2-(1,1-dioxo-1λ⁶-[1,2]thiazinan-2-yl)benzonitrile, (2.63 g, 11.14 mmol) was dissolved in ethanol (150 mL) and 1N HCl (13 mL). Added to this was 10% Pd—C (0.5 g) and the mixture shaken tinder H₂ at 55 psi for 24 h. Filtration through Celite followed by concentration gave the title compound as a white solid (2.93 g, 95% yield). ¹H-NMR (300 MHz, CD₃OD) δ ppm: 7.61-7.47 (4H, m), 4.30 (2H, q, J=13.7 Hz), 3.96-3.87 (1H, m), 3.49-3.36 (3H, m), 2.40-2.31 (2H, m), 2.05-1.96 (2H, m). LCMS [M+H]⁺ calcd for C₁₁H₁₇N₂SO₂: 241.10. found: 241.10.

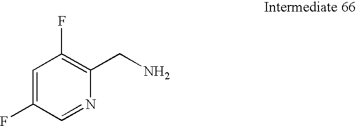

(3,5-Difluoropyridin-2-yl)methanamine hydrochloride. A mixture of 3,5-difluoropicolinonitrile (1.4 g, 10 mmol), conc. HCl (12 ml) and 10% Pd—C (200 mg) in 1:1 ethanol/tetrahydrofuran was shaken under a hydrogen atmosphere (50 psi) for 5 h. The reaction mixture was filtered and the ethanol removed in vacuo. The remaining solution was lyophilized to afford an off-white solid (2.16 g, 100% yield). LCMS (M+H) calcd for C₆H₇F₂N₂: 145.06. found: 145.12.

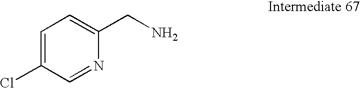

(5-Chloropyridin-2-yl)methanamine. A solution of 5-chloropicolinonitrile (3.8 g, 27.43 mmol), conc. HCl (3 mL) and 10% Pd—C (1.0 g) in ethanol (100 mL) was shaken under a hydrogen atmosphere (40 psi) for 2 h. The reaction mixture was filtered, concentrated and the resulting residue taken up in satd NaHCO₃ (50 mL) and extracted with CH₂Cl₂ (4×25 mL). The combined CH₂Cl₂ layers were dried (Na₂SO₄), filtered and concentrated to give the title compound as a yellow oil (2.0 g, 51% yield). LCMS (M+H) calcd for C₆H₈ClN₂: 143.04. found: 143.07. ¹HNMR (500 MHz, CDCl₃) δ ppm: 8.56-8.51 (1H, br d), 7.66-7.60 (1H, m), 7.28-7.14 (1H, m), 3.97 (2H, s), 1.72 (2H, s).

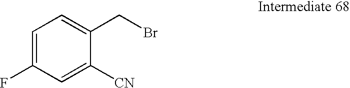

2-(Bromomethyl-5-fluorobenzonitrile. N₂ was passed through a mixture of 5-fluoro-2-methylbenzonitrile (28.51 g, 211 mmol), NBS (41.31 g, 232 mmol) and AIBN (2.5 g, 15 mmol) in CCl₄ (845 mL) for 10 min after which the reaction was heated at reflux for 8 h. After standing at room temperature overnight, the reaction mixture was filtered and the filter cake washed with CCl₄ (500 mL). The combined filtrate was evaporated to give a yellow oil. Flash chromatography (SiO₂) using 5-25% ethyl acetate/Hexanes as eluent afforded the title compound (29.74 g, 66% yield) as a pale yellow oil. ¹H NMR (500 MHz, CDCl₃) δ: 7.55 (1H, dd, J=8.6, 5.2 Hz), 7.37 (1H, dd, J=7.9, 2.8 Hz), 7.32-7.28 (1H, m), 4.61 (2H, s).

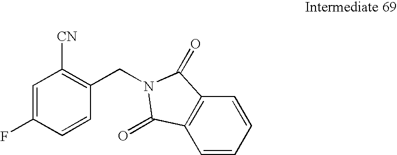

2-((1,3-Dioxoisoindolin-2-yl)methyl)-5-fluorobenzonitrile. To a stinted solution of intermediate 104, 2-(bromomethyl)-5-fluorobenzonitrile (29.72 g, 139 mmol) and phthalimide (32.69 g, 222 mmol) in dimethylformamide (300 mL) was added Cs₂CO₃ (67.87 g, 208 mmol). After stirring vigorously for 1 h, the reaction mixture was poured into water (1.2 L). The precipitated product was filtered, washed with water (600 mL) and methanol (150 mL) to give a white solid. The solid was taken up into 1 L of water/methanol (2:1) to which was added K₂CO₃ (12 g) and the mixture stirred at 40° C. After 30 min., the mixture was cooled and filtered. The filter cake was washed with water (500 mL), and dried under vacuum to afford the title compound (38.91 g, 94% yield) as a white powder. ¹H NMR (500 MHz, CDCl₃) δ: 7.89 (2H, dd, J=5.5, 3.1 HZ), 7.76 (5.5, 3.1 Hz), 7.41 (1H, dd, J=8.6, 5.2 Hz), 7.38 (1H, dd, J=7.9, 2.8 Hz), 7.24 (1H, td, J=8.2, 2.8 Hz), 5.06 (2H, s). LCMS (M+H) calcd for C₁₆H₁₀FN₂O₂: 281.07. found: 281.15.

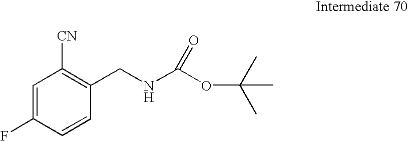

tert-Butyl 2-cyano-4-fluorobenzylcarbamate. A suspension of intermediate 105, 2-((1,3-dioxoisoindolin-2-yl)methyl)-5-fluorobenzonitrile, (5.6 g, 20 mmol) in dimethylformamide (20 mL) was warmed until it was dissolved. To this was added tetrahydrofuran (100 mL) and the mixture placed in a pre-heated (70° C.) oil bath. Hydrazine monohydrate was added to this and the reaction stirred for 8 h. The resulting white slurry was left at ambient temperature overnight. To this slurry was added di-tert-butyldicarbonate (6.55 g, 30 mmol) and the mixture stirred for 6 h at room temperature. The reaction mixture was diluted with ether (100 mL), filtered and the filtrate treated with activated carbon at 40° C. After filtration and concentration the crude product was purified by flash chromatography, using 20-30% ethyl acetate/Hexanes as eluent, to provide the title compound (2.88 g, 58% yield) as a light yellow powder. ¹H NMR (500 MHz, CDCl₃) δ: 9.46 (1H, br s), 7.61 (1H, dd, J==7.9, 2.1 Hz), 7.34 (1H, dd, J=8.2, 4.6 Hz), 7.22 (1H, td, J=8.6, 2.4 Hz), 4.71 (2H, s), 1.59 (9H, s). LCMS (M+H) calcd for C₁₃H₁₆FN₂O₂: 251.12. found: 251.22.

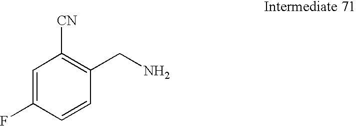

2-(Aminomethyl)-5-fluorobenzonitrile trifluoroacetic salt. A round-bottom flask was charged with intermediate 106, tert-butyl 2-cyano-4-fluorobenzylcarbamate, (1.9 g, 7.591 mmol) then treated with trifluoroacetic acid (20 ml) at room temperature. After 1 h, the reaction mixture was concentrated to give a yellow oil which was dissolved in CHCl₃ and re-concentrated to afford the title compound (2.01 g, 100% yield) as a pale yellow solid. LCMS (M+H) calcd for C₈H₈FN₂: 151.07. found: 151.08.

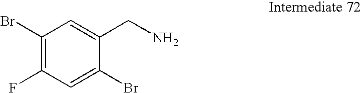

(2,5-Dibromo-4-fluorophenyl)methanamine. A solution of 2,5-dibromo-4-fluorobenzyl bromide (0.350 g, 1 mmol) in 7M N₃/MeOH was heated in a sealed tube at 100° C. for 2 h. The reaction mixture was cooled and concentrated to give a white solid which was dissolved in CH₂Cl₂ and treated with Et₃N (1 mL) then concentrated. The resulting residue was triturated with ethyl acetate (25 mL), filtered and concentrated to give the title compound (0.291 g) as a pale yellow oil. HRMS (M+H) calcd for C₇H₇Br₂FN: 283.94. found: 283.93.

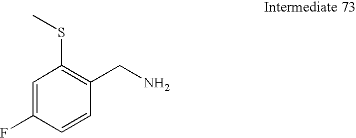

4-Fluoro-2-methylsulfanyl-benzylamine. Under N₂, 4-fluoro-2-(methylthio)benzonitrile (1.67 g, 0.1 mol) was dissolved in 20 mL tetrahydrofuran and treated with 10 mL 2M B₃.Me₂S. This was heated at 60° C. for 2 hrs. Heating was discontinued and 5 mL MeOH was cautiously added, followed by the cautious addition of 4 mL 6N HCl. Additional H₂O (20 mL) was added followed by ethyl acetate. The layers were separated. The aqueous layer was made basic with 1N NaOH and extracted with CH₂Cl₂. The extracts were dried (MgSO₄), filtered, concentrated and dried in vacuo to give 1.3 g of the title compound as a solid (Yield 76%). ¹H NMR (500 MHz, CDCl₃) δ ppm: 7.20-7.31 (1H, m) 6.90 (1H, dd, J=2.4 Hz) 6.75-6.86 (1H, m) 3.86 (2H, s) 2.47 (3H, s); LC/MS m/z 172.

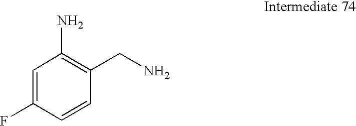

2-(Aminomethyl)-5-fluorobenzenamine hydrochloride. 2-Amino-4-fluorobenzonitrile (Fritz Hunziker et al. Eur. J. Med. Chem. 1981, 16, 391) (0.300 g, 1.68 mmol), was dissolved in acetic anhydride (5 mL) and the solution was stirred at 23° C. for 18 h. An additional portion of acetic anhydride (3 mL) was added to dissolve the N-(2-cyano-5-fluorophenyl)acetamide. Then palladium (10% on charcoal) (25 mg) was added and the mixture was agitated under H₂ (34 psi) for 72 h. The Pd—C was removed by filtration on Celite and the filtrate concentrated in vacuo to afford a bis-acetamide: LCMS (M+H)⁺ m/z 225. This was heated at reflux with HCl (6N, 10 mL) for 30 min. The acid was removed under reduced pressure to give a solid which was crystallized from MeOH-ether to afford the title compound (0.120 g, 51% yield). ¹H NMR (400 MHz, MeOD) δ ppm: 7.51 (1H, m), 6.96 (2H, m), 4.20 (2H, s).

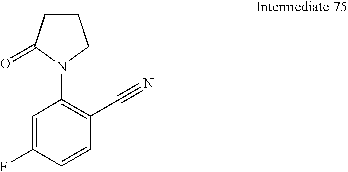

4-Fluoro-2-(2-oxopyrrolidin-1-yl)benzonitrile. A 48 mL pressure vessel containing 2-bromo-4-fluorobenzonitrile (1.00 g, 5.00 mmol), 2-pyrrolidinone (0.46 mL, 6.00 mmol), Cs₂CO₃ (2.28 g, 7.0 mmol) and 9,9-dimethyl-4,5-bis(diphenylphosphino)xanthene (xantphos) (0.231 g, 0.40 mmol) in dioxane (6 mL) was degassed with argon for 15 min. Pd₂ dba₃ was introduced and the reaction mixture heated at 105° C. for 48 h. The mixture was cooled, diluted with ethyl acetate or dioxane, and then filtered through Celite. The resulting mixture was concentrated in vacuo and subjected to column chromatography on silica gel with hexanes:ethyl acetate (3:7) gradient as the eluent to afford the title compound as a white solid (0.887 g, 87% yield): ¹H NMR (400 MHz, CDCl₃) δ ppm: 7.69 (1H, dd, J=5.8, 8.6 Hz), 7.22 (1H, dd, J=2.5, 9.6 Hz), 7.07 (1H, ddd, J=2.5, 7.6, 8.6 Hz), 3.96 (2H, t, J=7.0 Hz), 2.62 (2H, t, J=8.1 Hz), 2.30-2.22 (2H, m); LCMS (⁺ESI, M+H⁺) m/z 205.

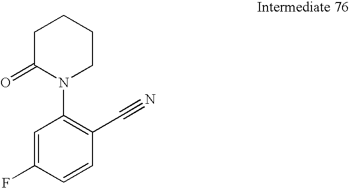

4-Fluoro-2-(2-oxopiperidin-1-yl)benzonitrile. The title compound can be prepared according to the procedure provided for intermediate 111 ¹H NMR (400 MHz, CDCl₃) δ ppm: 7.71 (1H, dd, J=5.7, 8.7 Hz), 7.14-7.06 (1H, m), 7.08 (1H, dd, J=2.4, 9.0 Hz), 3.65 (2H, t, J=5.7 Hz), 2.60 (2H, t, J=6.3 Hz), 2.05-1.95 (4H, m); LCMS (⁺ESI, M+H⁺) m/z 219.

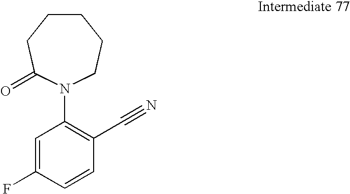

4-Fluoro-2-(2-oxoazepan-1-yl)benzonitrile. The title compound can be prepared according to the procedure provided for intermediate 111. ¹H NMR (400 MHz, CDCl₃) δ ppm: 7.68 (1H, dd, J=5.8, 8.6 Hz), 7.08 (1H, ddd, J=2.5, 7.6, 8.6 Hz), 7.01 (1H, dd, J=2.5, 9.0 Hz), 3.77-3.76 (2H, m), 2.75-2.72 (2H, m), 1.91-1.86 (6H, m); LCMS (⁺ESI, M+H⁺) m/z 233.

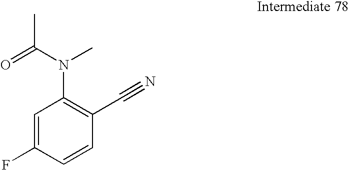

N-(2-Cyano-5-fluorophenyl)-N-methylacetamide. The title compound can be prepared according to the procedure provided for intermediate 111 ¹H NMR (400 MHz, CDCl₃) δ ppm: 7.79-7.75 (1H, m), 7.32-7.19 (1H, m), 7.10-7.07 (1H, m), 3.42 (0.6H, brs), 3.30 (2.4H, s), 2.32 (0.6H, brs), 1.91 (2.4H, s); LCMS (⁺ESI, M+H⁺) m/z 193; HPLC: 94% (220 nm).

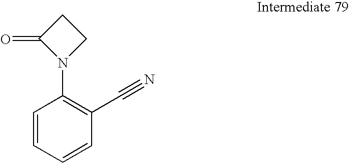

2-(2-Oxoazetidin-1-yl)benzonitrile. The title compound can be prepared according to the procedure provided for intermediate 111. ¹H NMR (400 MHz, DMSO-d₆) δ ppm: 8.02 (1H, d, J=8.4 Hz), 7.76 (1H, dd, J=1.5, 7.8 Hz), 7.69-7.65 (1H, m), 7.23 (1H, s), 4.04 (2H, t, J=4.8 Hz), 3.16 (2H, t, J=4.8 Hz). LCMS (⁺ESI, M+H⁺) m/z 173.

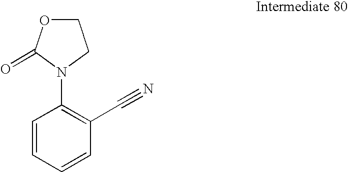

2-(2-Oxooxazolidin-3-yl)benzonitrile. The title compound can be prepared according to the procedure provided for intermediate 111. ¹H NMR (400 MHz, CDCl₃) δ ppm: 7.71 (1H, dd, J=1.5, 7.6 Hz), 7.68-7.63 (1H, m) 7.58 (1H, d, J=7.6 Hz), 7.38 (1H, dt, J=1.3, 7.6 Hz), 4.57 (2H, t, J=7.8 Hz), 4.21 (2H, t, J=7.8 Hz); LCMS (⁺ESI, M+H⁺) m/z 189.

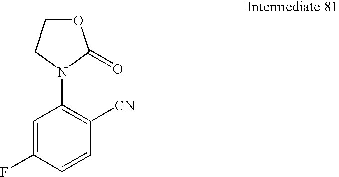

4-Fluoro-2-(2-oxooxazolidin-3-yl)benzonitrile. A 48 mL pressure vessel containing 2-bromo-4-fluorobenzonitrile (1.00 g, 5.00 mmol), 2-oxazolidone (0.390 g, 4.50 mmol), K₂CO₃ (0.970 g, 7.0 mmol) and xantphos (0.231 g, 0.40 mmol) in dioxane (10 mL) was degassed with argon for 15 inn. Pd₂ dba₃ (0.140 g, 0.15 mmol) was introduced and then the reaction mixture was heated at 70° C. for 18 h. The mixture was cooled, diluted with dioxane, and then filtered through Celite. The resulting mixture was concentrated in vacuo and subjected to column chromatography on silica gel with hexanes:ethyl acetate (1:1) to (3:7) gradient as the eluent to afford the title compound as a white solid (0.460 g, 50% yield): ¹H NMR (400 MHz, CDCl₃) δ ppm: 7.73 (1H, dd, J=5.8, 8.6 Hz), 7.43 (1H, dd, J=2.5, 9.6 Hz), 7.11 (1H, ddd, J=2.5, 7.5, 8.7 Hz), 4.60 (2H, t, J=7.1 Hz), 4.29 (2H, t, J=7.1 HJz); LCMS (⁺EST, M+H⁺) m/z 207.

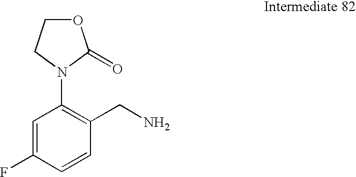

3-(2-(Aminomethyl)-5-fluorophenyl)oxazolidin-2-one hydrochloride. ¹H NMR (400 MHz, MeOD) δ ppm: 7.73 (1H, dd, J=6.0, 8.6 Hz), 7.43 (1H, dd, J=2.5, 9.5 Hz), 7.11 (1H, ddd, J=2.5, 7.5, 8.6 Hz), 4.64 (2H, t, J=7.7 Hz), 4.17 (2H, t, J=7.7 Hz), 4.14 (2H, s); LCMS (⁺EST, M+H⁺) m/z 211.

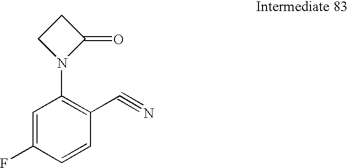

4-Fluoro-2-(2-oxoazetidin-1-yl)benzonitrile. The title compound can be prepared according to the procedure provided for intermediate 117 ¹H NMR (400 MHz, CDCl₃) δ ppm: 8.06 (1H, dd, J=10.7, 2.6 Hz), 7.58 (1H, dd, J=8.6, 6.3 Hz), 7.87 (1H, td, J=8.6, 2.5 Hz), 4.25 (2H, t, J=5.0 Hz), 3.26 (2H, t, J=5.0 Hz); LCMS (⁺ESI, M+H⁺) m/z 191.

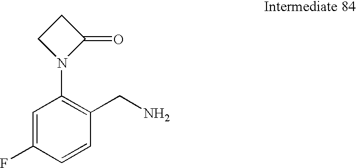

1-(2-(Aminomethyl)-5-fluorophenyl)oxazolidin-2-one hydrochloride. ¹H NMR (400 MHz, DMSO/D₂0) δ ppm: 7.54 (1H, dd, (t), J=8.6 Hz), 7.25 (1H, dd, J=10.8, 2.5 Hz), 7.17 (1H, td, J=8.6, 2.5 Hz), 4.12 (2H, s), 3.79 (2H, t, J=4.6 Hz), 3.09 (2H, t, J=4.6 Hz); LCMS (⁺ESI, M+H⁺) m/z 195.

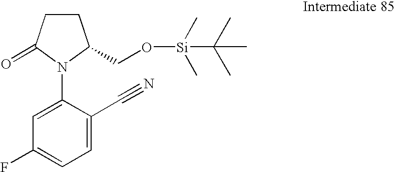

(R)-2-(2-((tert-Butyldimethylsilyloxy)methyl)-5-oxopyrrolidin-1-yl)-4-fluorobenzonitrile. The title compound can be prepared according to the procedure provided for intermediate 111. ¹H NMR (400 MHz, CDCl₃) δ ppm: 7.68 (1H, dd, J=5.8, 8.8 Hz), 7.19 (1H, dd, J=25, 9.1 Hz), 7.11-7.07 (1H, m), 4.46-4.42 (1H, m), 3.55 (2H, d, J=3.3 Hz), 2.72-2.52 (2H, m), 2.43-2.33 (1H, m), 2.09-2.01 (1H, m), 0.81 (9H, s), −0.04 (3H, s), −0.07 (3H, s); LCMS (⁺ESI, M+H⁺) m/z 349.

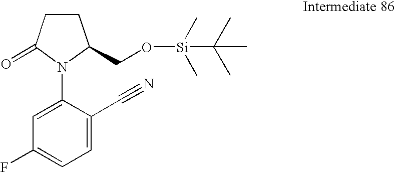

(S)-2-(2-((tert-Butyldimethylsilyloxy)methyl)-5-oxopyrrolidin-1-yl)-4-fluorobenzonitrile. The title compound can be prepared according to the procedure provided for intermediate 111. ¹H NMR (400 MHz, CDCl₃) δ ppm: 7.68 (1H, dd, J=5.8, 8.6 Hz), 7.19 (1H, dd, J=2.5, 9.4 Hz), 7.11-7.07 (1H, m), 4.46-4.43 (1H, m), 3.55 (2H, c, J=3.3 Hz), 2.72-2.52 (2H, m), 2.43-2.33 (1H, m), 2.09-2.01 (1H, m), 0.81 (9H, s), −0.04 (3H, s), −0.07 (3H, s); LCMS (⁺ESI, M+H⁺) m/z 349.

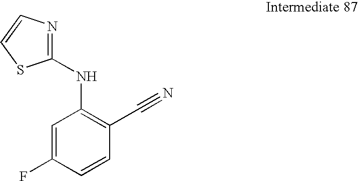

4-Fluoro-2-(thiazol-2-ylamino)benzonitrile. The title compound can be prepared according to the procedure provided for intermediate 111. ¹H NMR (400 MHz, DMSO-d₆) δ ppm: 9.21 (1H, s), 8.39-8.35 (1H, m), 7.97 (1H, d, J=5.0 Hz), 7.23-7.13 (3H, m); LCMS (⁺ESI, M+H⁺) m/z 220.

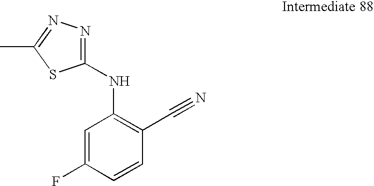

4-Fluoro-2-(5-methyl-1,3,4-thiadiazol-2-ylamino)benzonitrile. The title compound can be prepared according to the procedure provided for intermediate 111. ¹H NMR (400 MHz, DMSO-d₆) δ ppm: 8.30 (1H, dd, J=6.5, 8.8 Hz), 7.96 (1H, s), 7.26-7.19 (2H, m), 2.64 (3H, s); LCMS (⁺ESI, M+H⁺) m/z 235.

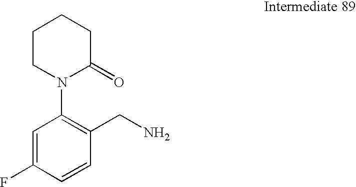

1-(2-(Aminomethyl)-5-fluorophenyl)piperidin-2-one hydrochloride salt. To a stirred solution of intermediate 112, 4-fluoro-2-(2-oxopiperidin-1-yl)benzonitrile (150 mg, 0.69 mmol) in H₂O (10 mL) was added ethanol (10 mL) 10% palladium on charcoal (50 mg) and 1N HCl (2.1 mL, 20.6 mmol). The reaction was shaken in a Parr system under H₂ (40 psi) for 1 h. Then the Pd/C catalyst was removed by filtration on Celite and the filtrate was concentrated in vacuo to yield a solid. Toluene (2×50 mL) was added to the solid and the solution was evaporated in vacuo. LCMS (M+H)⁺ m/z 170.

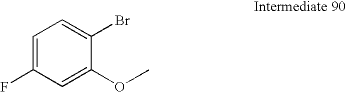

1-Bromo-4-fluoro-2-methoxybenzene. To a mixture of 2-bromo-5-fluorophenol (10 g, 50.8 mmol) and iodomethane (11.2 g, 78.7 mmol) in dimethylformamide (100 mL) was added potassium carbonate (10.9 g, 79 mmol) and the mixture stirred at room temperature for 3 hrs. The mixture was diluted with water (100 mL) and extracted with ether (50 mL×3). The combined extracts were washed with brine, dried over anhydrous magnesium sulfate, and concentrated in vacuo to obtain 11.3 g of 1-bromo-4-fluoro-2-methoxybenzene as an amber colored oil.

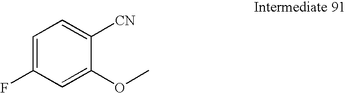

4-Fluoro-2-methoxybenzonitrile. To a solution of intermediate 126, 1-bromo-4-fluoro-2-methoxybenzene (9.0 g) in N-methylpyrrolidone (100 mL, Sure Seal; Aldrich) was added CuCN (6.6 g, 73.7 mmol, 1.8 eq.; Aldrich), and the mixture stirred at 180° C. under anhydrous nitrogen for 5.5 hrs. After cooling, 14% aqueous NH₄OH (330 mL) was added and stirring continued for 45 min at room temperature. The mixture was extracted with ether (100 mL×3), and the combined extracts washed sequentially with dilute aqueous NH₄OH, dilute HCl and brine, then dried (MgSO₄), and concentrated to provide the title compound (5.2 g, Yield 85% in 2 steps) as a white solid: ¹H NMR (CDCl₃, 500 MHz) o ppm: 3.91 (3H, s, OMe), 6.69 (1H, dd, J=2.3 Hz, J=10.5 Hz, Ar—H), 6.72 (1H, dt, J=2.5 Hz, J=J=8.0 Hz, Ar—H), 7.55 (1H, dd, J=6.5 Hz, J=8.5 Hz, Ar—H); ¹³C NMR (CDCl₃, 125.8 Hz) δ ppm: 56.49, 98.16, 100.06, 100.27, 108.31, 108.50, 115.83 135.37, 135.46, 163.25, 163.34 165.47, 167.50. An analytical sample was obtained by trituration with ether. Anal. calcd for C₈H₆FNO: C, 63.57; H, 4.00; N, 9.26. found: C, 63.36; H, 3.91; N, 9.16.

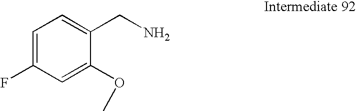

4-Fluoro-2-methoxybenzylamine hydrochloride. To a mixture of intermediate 127, 4-fluoro-2-methoxybenzonitrile, (800 mg, 5.3 mmol) and conc.HCl (0.53 mL, 6.36 mmol, 1.2 eq.) in ethanol (20 mL) was added 10% Pd—C (100 mg; Aldrich), and the mixture hydrogenated at 1 atm hydrogen for 15 hrs at room temperature. To this mixture was added an additional amount of conc.HCl (1 mL) and 10% Pd—C (200 mg) and the reaction allowed to continue for another 40 hrs. The mixture was filtered through Celite and the filtrate concentrated in vacuo to dryness. The residue was triturated with ether to provide the title compound (895 mg, Yield 88%) as a white powder: ¹H NMR (CDCl₃, 500 MHz) δ ppm: 3.84 (3H, S, OMe), 3.91 (2H, d, J=5.5 Hz, N—CH₂), 6.81 (1H, dt, J=2.5 Hz, J=J=8.5 Hz, Ar—H), 6.99 (1H, dd, J=2.5 Hz, J=11.3 Hz, Ar—H), 7.47 (1H, dd, J=7 Hz, 3=8.5 Hz, Ar—H); ¹³C NMR (CDCl₃, 125.8 Hz) δ ppm: 36.76, 56.03, 99.30, 99.51 106.28, 106.45, 117.93, 117.95, 131.60, 131.69, 158.56, 158.64, 162.28, 164.22. HRMS (ESI) calcd for C₈H₁₁FNO (M+H) 156.0825. found 156.0830.

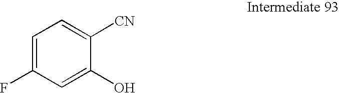

4-Fluoro-2-hydroxybenzonitrile. A mixture of intermediate 127, 4-fluoro-2-methoxybenzonitrile, (4.53 g, 30 mmol) and AlCl₃ (5.0 g, 37.6 mmol; Aldrich) in anhydrous toluene (30 mL) was stirred at approximately 130° C. for 18 hrs. After cooling, ice water (˜50 ml) was added and the resulting mixture extracted with ether (20 mL×2). The combined extracts were washed sequentially with water and brine, then dried (MgSO₄), and concentrated in vacuo to provide the title compound (3.90 g, 28.5 mmol, Yield 95%) as a white solid: ¹H NMR (DMSO-d6, 300 MHz) δ ppm: 6.74-6.84 (2H, m, Ar—Hs), 7.71 (1H, dd, J=7 Hz, J=8.5 Hz, Ar—H), 11.64 (1H, s, OH); ¹³C NMR (DMSO-d6, 75.5 Hz) δ ppm: 95.13 102.45, 102.78, 106.53, 106.83 115.53, 134.68, 134.84, 161.41, 161.58, 163.00, 166.35. HRMS (ESI−) calcd for C₇H₃NOF (M−H) 136.0199. found 136.0199.

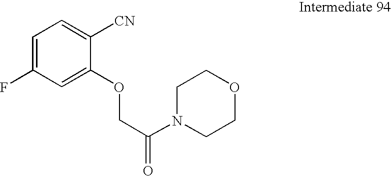

4-Fluoro-2-(2-morpholino-2-oxoethoxy)benzonitrile. To a solution of intermediate 129, 4-fluoro-2-hydroxybenzonitrile, (685 mg, 5 mmol) in dimethylformamide (8 mL, Sure Seal; Aldrich) was added NaH (200 mg, 5 mmol; 60% oil dispersion; Aldrich), and the mixture stirred for 5 min under an anhydrous nitrogen atmosphere. To this was added 4-(2-chloroacetyl)morpholine (900 mg, 5.5 mmol, 1.1 eq.; Avocado Organics), and stirring continued at room temperature for 21 hrs. The reaction was quenched by careful addition of water (30 mL). The resulting mixture was extracted with CH₂Cl₂ (25 mL×2). The combined extracts were washed with brine, dried (MgSO₄) and concentrated. The residue was triturated to obtain 1.10 g (4.17 mmol, Yield 83%) of the title compound as a white solid: ¹H NMR (CDCl₃, 500 MHz) δ ppm: 3.63 (2H, t, J=4 Hz, NCH₂), 3.67 (1H, m, OCH), 3.72 (1H, m, OCR), 4.86 (2H, s, OCH₂), 6.80-6.86 (2H, m, Ar—Hs), 7.61 (1H, dd, J=8.5 Hz, 6.1 Hz, Ar—H); ¹³C NMR (CDCl₃, 125.77 Hz) δ ppm: 42.63, 46.04, 66.80, 68.33, 98.45, 98.47, 101.57, 101.79, 109.56, 109.74, 115.42, 135.48, 135.57, 161.26, 161.35, 114.79, 165.23, 167.28. HRMS calcd for C₁₃H₁₄N₂O₃F (M+H) 265.0988. found 265.0998.

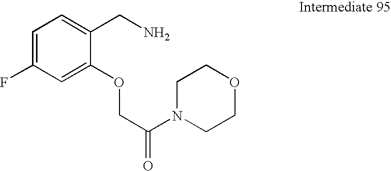

2-(2-(Aminomethyl)-5-fluorophenoxy-1-morpholinoethanone hydrochloride. A solution of intermediate 130, 4-fluoro-2-(2-morpholino-2-oxoethoxy)benzonitrile, (500 mg, 1.89 mmol) in warm ethanol (30 mL) and ethyl acetate (30 mL) was mixed with conc.HCl (0.32 mL, 3.78 mmol, 2 eq.). To this was added 100% Pd—C (100 mg; Aldrich), and the mixture was hydrogenated at 1 atm of hydrogen for 20 hrs at room temperature. To this mixture was added an additional amount of 10% Pd—C (50 mg) and stirring continued for another 7 hrs. The mixture was filtered through Celite and the filtrate concentrated in vacuo to dryness. The residue was triturated with ethyl acetate, then with ethanol to obtain the title compound (168 mg, Yield 29%) as an off-white powder: ¹H NMR (CD₃OD, 500 MHz) δ ppm: 3.55 (2H, t, J=5 Hz, NCH₂), 3.62 (2H, t, J=5 Hz, NCH₂), 3.70 (2H, t, J=5 Hz, OCH₂), 3.75 (2H, t, J=5 Hz, OCH₂), 4.17 (2H, s, NCH₂), 5.17 (2H, s, OCH₂), 6.82 (1H, dt, J=2.5, 8.5 Hz, Ar—H), 7.05 (1H, dd, J=2.5, 10.5 Hz, Ar—H), 7.43 (1H, dd, J=6.5, 8.5 Hz, Ar—H); ¹³C NMR (CD₃OD, 125.77 Hz) δ ppm: 39.40, 42.49, 44.97, 66.11, 66.46, 66.59, 101.38, 101.59, 108.40, 108.57, 118.40, 132.53, 132.62, 158.43, 158.52, 63.87, 165.83, 168.27. HRMS (ESI) calcd for C₁₃H₁₈N₂O₃F (M+H) 269.1301. found 269.1301.

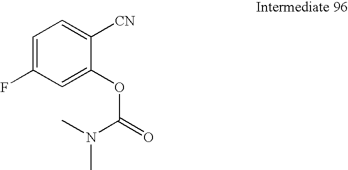

Dimethyl-carbamic acid 2-cyano-5-fluoro-phenyl ester. Under N₂, a stirred solution of intermediate 129, 4-fluoro-2-hydroxybenzonitrile (685 mg, 5.00 mmol), dimethylcarbamoyl chloride, and triethylamine (606 mg, 6 mmol) in dichloroethane (10 mL) was heated at reflux for 20 hrs. The cooled mixture was diluted with dichloroethane (10 mL) washed with water, and brine. The organic layer was separated, dried (Na₂SO₄), concentrated, and the residue purified by column chromatography (SiO₂, 5% ethyl acetate-CH₂Cl₂) to provide 700 mg (Yield 67%) of the title compound as a white crystalline solid: ¹H NMR (CDCl₃, 500 MHz) δ ppm: 3.03 (3H, s, NMe), 3.15 (3H, s, NMe), 6.99 (1H, dt, J=2.5 Hz, 8.5 Hz, Ar—H), 7.23 (1H, dd, J=2.5 Hz, 9.5 Hz, Ar—H), 7.61 (1H, dd, J=9 Hz, 6 Hz, Ar—H); ¹³C NMR (CDCl₃, 125.77 Hz) δ ppm: 36.76, 37.06, 102.84, 102.86, 111.59, 111.79, 113.24, 113.42, 114.99, 134.36, 134.45, 152.54, 155.06, 155.16, 164.26, 166.31. HRMS (ESI) calcd for C₁₀H₁₀N₂O₂F (M+H) 209.0726. found 209.0722.

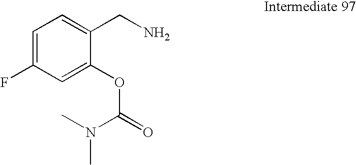

Dimethyl-carbamic acid 2-aminomethyl-5-fluoro-phenyl ester hydrochloride. To a solution of intermediate 132, dimethyl-carbamic acid 2-cyano-5-fluoro-phenyl ester, (340 mg, 1.63 mmol) in ethyl acetate (20 mL) and ethanol (20 mL), was added conc.HCl (0.4 mL) and 10% Pd—C (100 mg) and the mixture hydrogenated in a Parr Shaker at 55 psi of hydrogen for 20 hrs. The reaction mixture was filtered through Celite, and the filtrate concentrated in vacuo to give an oil which was partitioned between ethyl acetate (10 mL) and water (10 mL). After separation, the aqueous phase was washed with additional ethyl acetate (5 mL). The combined extracts were concentrated in vacuo to dryness. The residual oil was triturated with ether to provide 145 mg (Yield 38%) of the title compound, as a tan powder: ¹H NMR (CD₃OD, 500 MHz) δ ppm: 3.06 (3H, s, NMe), 3.21 (3H, s, NMe), 4.11 (2H, s, NCH₂), 7.13 (2H, m, Ar—Hs), 7.60 (1H, m, Ar—H); ¹³C NMR (CD3OD, 125.77 Hz) δ ppm: 36.03, 36.25 37.58, 110.79, 110.99, 113.26, 113.43, 122.32, 132.18, 132.25, 151.55, 154.72, 162.69, 164.67. HRMS (ESI) calcd for C₁₀H₁₃N₂O₂F (M+H) 213.1039. found 213.1039.

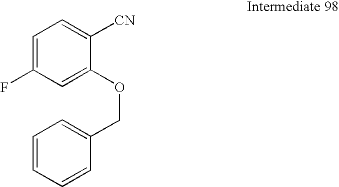

2-(Benzyloxy)-4-fluorobenzonitrile. Benzyl alcohol (13 mL, 125 mmol) was slowly added to a stirred suspension of NaH (95%, 2.86 g, 113 mmol) in toluene (200 mL) at room temperature. After 30 min, 2,4-difluorobenzonitrile (15.3 g, 110 mmol; Aldrich) was added all at once and stirring continued overnight (18 h). After this, the reaction mixture was washed with water (2×25 mL) and brine (25 ml). The organic layer was dried (Na₂SO₄), filtered and concentrated to give a white slurry which was triturated with hexanes and filtered to afford the title compound as a white solid (20.34 g, 81% yield), ¹H NMR (500 MHz, CDCl₃): 7.59-7.55 (1H, m), 7.45-7.34 (5H, m), 6.75-6.71 (2H, m), 5.19 (2H, s); ¹³C NMR (125.76 MHz, DMSO-d6) δ ppm: 71.16, 98.75, 101.54, 101.75, 108.66, 108.84, 115.83, 127.16, 128.58, 128.94, 135.03, 135.44, 135.54, 162.22, 162.31, 165.26, 167.29. LCMS calcd for C₁₄H₁₁FNO: 228.2. found: 228.0.

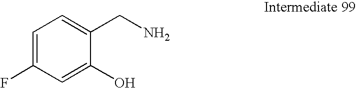

2-Hydroxy-4-fluoro-benzylamine hydrochloride. A solution of intermediate 134, 2-(benzyloxy)-4-fluorobenzonitrile, (9.03 g, 39.7 mmol) in ethanol (100 mL) and ethyl acetate (100 mL) was stirred with 10% palladium on carbon (1.67 g) and concentrated hydrochloric acid (12 mL, 144 mmol) under a hydrogen atmosphere (60 psi) for four days. The catalyst was removed by filtration through Celite, and the filtrate was concentrated. The crude product was triturated with ether and the resulting solid collected by filtration to give the title compound (5.24 g, 74% yield) as a pale orange solid. ¹H NMR (500 MHz, DMSO-D6) δ ppm: 10.81 (1H, s), 8.18 (3H, s), 7.36 (1H, t, J=17.3 Hz), 6.79 (1H, dd, J=10.8, 2.6 Hz), 6.66 (1H, dt, J=8.5, 2.3 Hz), 3.90 (2H, d, J=5.2 Hz).

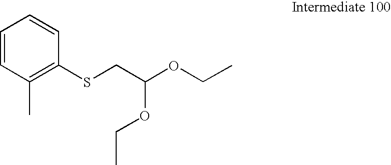

(2,2-Diethoxyethyl)(o-tolyl)sulfane. In ethanol (50 mL) was dissolved sodium metal (1.6 g, 66 mmol) at 23° C. 2-Methylbenzenethiol (8.1 mL, 68 mmol) was slowly added to this solution, followed by bromoacetaldehyde diethylacetal (9.50 mL, 63 mmol). The reaction mixture was stirred at reflux for 18 h. The solvent was then evaporated in vacuo and the residue was washed with H₂O (100 mL) and extracted with ether (100 mL). The organic solution was dried (MgSO₄), concentrated in vacuo and purified by distillation to afford the title compound (13.48 g, 82% yield): ¹H NMR (400 MHz, CDCl₃) δ ppm: 7.33 (1H, d, J=7.9 Hz), 7.16-7.08 (3H, m), 4.65 (1H, t, J=5.6 Hz), 3.66 (2H, q, J=7.0 Hz), 3.55 (2H, q, J=7.0 Hz), 3.09 (2H, d, J=5.6 Hz), 2.38 (3H, s), 1.20 (6H, t, J=7.0 Hz). LCMS (M+H)⁺ m/z 241 (t=2.6 min.).

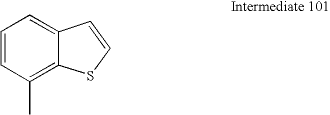

7-Methylbenzo[b]thiophene. To a solution of intermediate 136, (2,2-diethoxyethyl)(o-tolyl)sulfane (0.58 g, 2.41 mmol) in chlorobenzene (20 mL) was added polyphosphoric acid. The reaction mixture was stirred at reflux for 18 h. Water (100 mL) was then added and the organic material was extracted with CH₂Cl₂ (2×50 mL). The organic solution was dried (MgSO₄) and concentrated in vacuo to afford 335 mg (94% yield) of the title compound: ¹H NMR (400 MHz, CDCl₃) δ: 7.68 (1H, d, J=7.8 Hz), 7.43 (1H, d, J=5.4 Hz), 7.36 (1H, d, J=5.4 Hz) 7.30 (1H, dd, J=7.8, 7.1 Hz), 7.14 (1H, d, J=7.1 Hz), 2.58 (3H, s); LCMS (M+H)⁺ m/z 148.

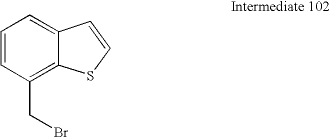

7-(Bromomethyl)benzo[b]thiophene. To a solution of intermediate 137, 7-methylbenzo[b]thiophene (1.0 g, 6.5 mmol) in CCl₄ (20 mL) was added benzoyl peroxide (1.1 g, 4.54 mmol) followed by portion wise addition of NBS (1.15 g, 6.5 mmol). The reaction mixture stirred at reflux while irradiating with a 250 W lamp. The reaction mixture was stirred at reflux for 3 h. The solution was cooled, filtered and the solvent evaporated in vacuo. The residue was subjected to column chromatography on silica gel with hexanes as the element to afford the title compound (0.570 g, 33% yield): ¹H NMR (400 MHz, CDCl₃) δ: 7.80 (1H, dd, J=7.8, 1.7 Hz), 7.49 (1H, d, J=5.4 Hz), 7.40-7.33 (3H, m), 4.78 (2H, s). LCMS (M+H)⁺ m/z 209.

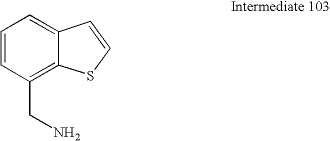

Benzo[b]thiophen-7-ylmethanamine hydrochloride. To intermediate 138, 7-(bromomethyl)benzo[b]thiophene (0.20 g, 0.96 mmol) was added a methanolic solution saturated with ammonia (30 mL). The reaction mixture was heated in a steal bomb at 70° C. for 18 h. The solvent was evaporated in vacuo and the residue was dissolved in MeOH (10 mL). HCl (1M in ethanol, 1 mL) was added to the solution and the solvents were removed in vacuo to afford the title compound (0.177 g, 99% yield); LCMS (M+H)⁺ m/z 164.

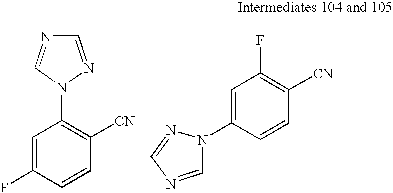

4-Fluoro-2-(1H-1,2,4-triazol-1-yl)benzonitrile and 4-(1H-1,2,4-triazol-1-yl)-2-fluorobenzonitrile. To a solution of 2,4-difluorobenzonitrile (10 g, 72 mmol) dissolved in THF (20 mL), and DMF (40 mL) was added 1,2,4-triazole sodium (6.3 g, 70 mmol) and the mixture was stirred at 90° C. for 3 h, filtered and concentrated. The residue was adsorbed onto Silica gel and purified by flash chromatography eluting with 0%-10%-30% EtOAc/hexanes to give the 4-triazolyl isomer as colorless needles (2.46 g, 18%) and the 2-substituted isomer as a white solid (0.7455 g, 6%).

4-Fluoro-2-(1H-1,2,4-triazol-1-yl)benzonitrile. ¹H NMR (500 MHz, CDCl₃) δ: 8.89 (1H, s), 8.19 (1H, s), 7.85 (1H, dd, J=8.7, 5.6 Hz), 7.60 (1H, dd, J=8.8, 2.4 Hz), 7.28-7.24 (1H, m). LCMS (M+H) calcd for C₉H₆N₄F: 189.05. found: 189.13.

4-(1H-1,2,4-triazol-1-yl)-2-fluorobenzonitrile. ¹H NMR (500 MHz, CDCl₃) δ: 8.66 (1H, s), 8.15 (1H, s), 7.79 (1H, dd, J=8.5, 6.7 Hz), 7.69 (1H, dd, J=9.5, 1.8 Hz), 7.65-7.63 (1H, m). LCMS (M+H) calcd for C₉H₆N₄F: 189.05. found: 189.13.

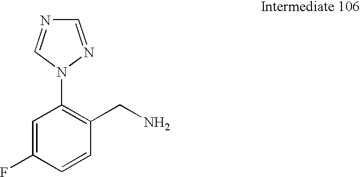

(4-Fluoro-2-(1H-1,2,4-triazol-1-yl)phenyl)methanamine hydrochloride. 4-Fluoro-2-(1H-1,2,4-triazol-1-yl)benzonitrile (2.46 g, 13.13 mmol) was dissolved in hot ethanol (150 mL). Aqueous HCl (15 ml, 1N) was added followed by 10% Pd/C (200 mg). The mixture was shaken under H₂ at 55 psi for 4 h, filtered over celite and concentrated. The residue was partitioned between EtOAc and water. The aqueous phase was lyophilized to give the benzyl amine as a white powder (2.96 g, 99%). ¹H NMR (500 MHz, CD₃OD) δ: 9.51 (1H, s), 8.63 (1H, s), 7.85 (1H, dd, J=8.5, 5.8 Hz), 7.68 (1H, dd, J=8.8, 2.4 Hz), 7.49 (1H, td, J=8.3, 2.4 Hz), 4.20 (2H, s). LCMS (M+H) calcd for C₉H₁₀N₄F: 193.08. found: 193.16.

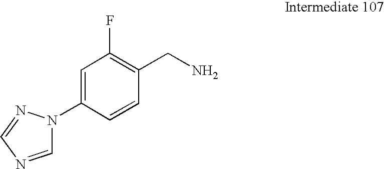

(2-Fluoro-4-(1H-1,2,4-triazol-1-yl)phenyl)methanamine hydrochloride. This compound was prepared (79% yield) following the procedure for (4-Fluoro-2-(1H-1,2,4-triazol-1-yl)phenyl)methanamine hydrochloride using (2-Fluoro-4-(1H-1,2,4-triazol-1-yl)benzonitrile. ¹H NMR (500 MHz, CD₃OD) δ: 9.25 (1H, s), 8.46 (1H, s), 7.80 (1H, dd, J=8.6, 5.8 Hz), 7.64 (1H, dd, J=8.8, 2.4 Hz), 7.44 (1H, td, J=8.3, 2.6 Hz), 4.17 (2H, s). LCMS (M+H) calcd for C₉H₁₀N₄F: 193.08. found: 193.16.

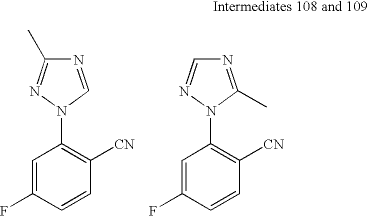

4-Fluoro-2-(3-methyl-1H-1,2,4-triazol-1-yl)benzonitrile and 4-fluoro-2-(5-methyl-1H-1,2,4-triazol-1-yl)benzonitrile. A solution of 2,4-difluorobenzonitrile (7.07 g, 50.8 mmol) and 3-methyl-1H-1,2,4-triazole (4.22 g, 50.8 mmol) in N,N-dimethylformamide (45 ml) was treated with powdered anhydrous potassium carbonate (10 g) and the resulting mixture was stirred at 22° C. for 18 h. The solid was then filtered and the filtrate was concentrated in vacuo. The residue was diluted with ethyl acetate, washed with water and brine, dried over anhydrous magnesium sulfate and concentrated. The mixture containing the 2 and 4-triazolyl-benzonitriles was purified by a combination of chromatography on silica gel (elution gradient of ethyl acetate in hexane) and on reversed phase silica gel to give 1.86 g (18% yield) of 4-Fluoro-2-(3-methyl-1H-1,2,4-triazol-1-yl)benzonitrile and 0.526 g (5% yield) of 4-fluoro-2-(5-methyl-1H-1,2,4-triazol-1-yl)benzonitrile.

4-Fluoro-2-(3-methyl-1H-1,2,4-triazol-1-yl)benzonitrile. White crystals (ethyl acetate/hexanes). mp 117-118° C. ¹HNMR 400 MHz (CDCl₃) δ (ppm): 2.54 (3H, s, CH₃), 7.24 (1H, m, CH), 7.62 (1H, dd, J=2.5 Hz and J=9.1 Hz, CH), 7.84 (1H, dd, J=5.6 Hz and J=8.6 Hz, CH), 8.82 (1H, s, CH). Anal. Calcd for C₁₀H₇FN₄: C, 59.40; H, 3.49; N, 27.71. Found: C, 59.25; H, 3.32; N, 27.81.

4-fluoro-2-(5-methyl-1H-1,2,4-triazol-1-yl)benzonitrile. White crystals (ethyl acetate-hexane). mp 120-121° C. ¹HNMR 400 MHz (CDCl₃) δ (ppm): 2.56 (3H, s, CH₃), 7.30 (1H, dd, J=2.5 Hz and J=8.1 Hz, CH), 7.39 (1H, m, CH), 7.91 (1H, dd, J=5.5 Hz and J=8.6 Hz, CH), 8.06 (1H, s, CH). Anal. Calcd for C₁₀H₇FN₄: C, 59.40; H, 3.49; N, 27.71. Found: C, 59.35; H, 3.70; N, 27.77.

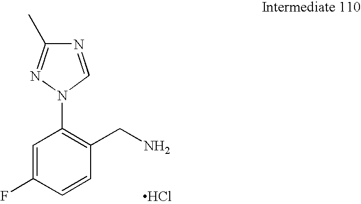

(4-Fluoro-2-(3-methyl-1H-1,2,4-triazol-1-yl)phenyl)methanamine hydrochloride salt. Hydrogenation of 4-fluoro-2-(3-methyl-1H-1,2,4-triazol-1-yl)benzonitrile (0.680 g, 3.36 mmol) gave 0.720 g (88% yield) of the title hydrochloride salt as a white solid. ¹HNMR 400 MHz (DMSO-d₆) δ (ppm): 2.40 (3H, s, CH₃), 4.02 (2H, m, NCH₂), 7.50 (1H, m, CH), 7.62 (1H, dd, J=2.8 Hz and J=9.3 Hz, CH), 7.84 (1H, dd, J=6.1 Hz and J=9.1 Hz, CH), 9.00 (1H, s, CH). HRMS (ESI⁺) calculated for C₁₀H₁₂FN₄ [M+H⁺]: 207.1046. found: 207.1047.

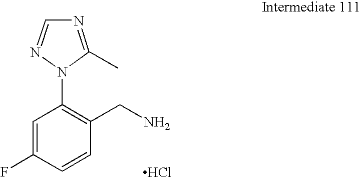

(4-Fluoro-2-(5-methyl-1H-1,2,4-triazol-1-yl)phenyl)methanamine hydrochloride salt. Hydrogenation of 4-fluoro-2-(5-methyl-1H-1,2,4-triazol-1-yl)benzonitrile (0.244 g, 1.20 mmol) gave 0.290 g (100% yield) of the title hydrochloride salt as a white solid. ¹HNMR 400 MHz (DMSO-d₆) δ (ppm) 2.42 (3H, s, CH₃), 3.78 (2H, m, NCH₂), 7.58 (1H, m, CH), 7.67 (1H, dd, J=2.8 Hz and J=9.3 Hz, CH), 7.90 (1H, dd, J=6.0 Hz and J=8.6 Hz, CH), 8.22 (1H, s, CH). HRMS (ESI⁺) calculated for C₁₀H₁₂FN₄ [M+H⁺]: 207.1046. found: 207.1041.

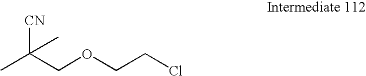

3-(2-Chloroethoxy)-2,2-dimethylpropanenitrile: To a solution of LDA (0.14 mol) in 100 mL THF at −30 C under N₂ was added drop-wise isobutyronitrile (9.7 g, 0.14 mol) in 40 mL THF over 20 min. After 20 min, a solution of 1-chloro-2-(chloromethoxy)ethane (18.1 g, 0.14 mol) in 50 mL THF was added drop-wise and the temperature was allowed to gradually rise to room temperature and stirred for 5 h. This was treated with 200 mL of water and Et₂O and the layers separated. The aqueous layer was extracted further with Et₂O. The combined extracts were washed with brine, dried over Na₂SO₄, filtered and concentrated to leave 23 g of crude product as a yellow oil. This was purified by silica chromatography using 9:1 hexanes/CH₂Cl₂ to 4:1 hexanes/CH₂Cl₂ as eluants. This yielded 7.4 g (32%) of intermediate 112 as an oil. ¹H NMR (300 MHz, CDCl₃) δ: 1.33 (s, 6H), 3.43 (s, 2H), 3.61 (t, J=5.7 Hz, 2H), 3.77 (t, J=5.9 Hz, 2H). LC/MS (M+H): 162.

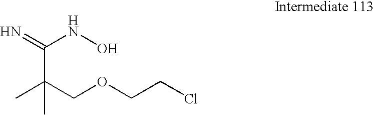

3-(2-Chloroethoxy)-N-Hydroxy-2,2-dimethylpropanamidine: Intermediate 112 (6.1 g, 37.7 mmol) was placed together with 50% aqueous hydroxylamine (3.1 g, 37.7 mmol) in 60 mL EtOH and warmed at 75-80 C with stirring for 18 h. The solution was concentrated and then concentrated further with EtOH and vacuum dried to leave (9.4 g, ˜80% pure) of intermediate 113 as a gum. ¹H NMR (300 MHz, CDCl₃) δ: 1.17 (s, 6H), 3.40 (s, 2H), 3.58-3.73 (m, 4H), 5.16 (s, 2H). LC/MS (M+H): 195.

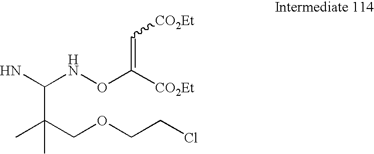

A solution of intermediate 113 (7.4 g, 37.7 mmol) in 70 mL EtOH and 10 mL H₂O was treated with diethyl acetylenedicarboxylate (6.4 g, 37.7 mmol). This was stirred for 1 h at room temperature and concentrated. The residue was dissolved in EtOAc and washed with water and brine. The EtOAc solution was dried over Na₂SO₄, filtered and concentrated to leave 14 g of a yellow oil. This was purified by chromatography on silica using 3:1 hexanes/EtOAc to give 5 g (36%) of intermediate 114 as clear oil. ¹H NMR (300 MHz, CDCl₃) δ: 1.15 (s, 6H), 1.16-1.39 (m, 6H), 3.41 (s, 2H), 3.56-3.77 (m, 4H), 4.05-4.20 (m, 2H), 4.21-4.37 (m, 2H), 5.30-5.45 (m, 1H), 5.62 (s, 0.5H), 5.64-5.75 (m, 1H), 5.77 (s, 0.5H). LC/MS (M+H): 365.

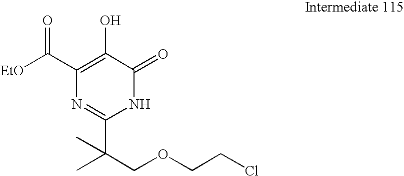

Intermediate 114 was dissolved in 150 mL 1,2,4-trimethylbenzene and heated at 155-160° C. for 2.5 hrs with stirring under N₂. The solvent was evaporated at reduced pressure and the residue was dissolved in EtOAc and extracted 2× with dil NaHCO₃. The aqueous extracts were acidified with HCl and extracted with CH₂Cl₂. After drying (MgSO₄), filtration and concentration provided intermediate 115 (1.9 g, 43%) as white solid. ¹H NMR (300 MHz, CDCl₃) δ: 1.31 (s, 6H), 1.41 (t, J=7.1 Hz, 3H), 3.54 (s, 2H), 3.63-3.72 (m, 2H), 3.75-3.83 (m, 2H), 4.42 (q, J=7.3 Hz, 2H). LC/MS (M+H): 319.

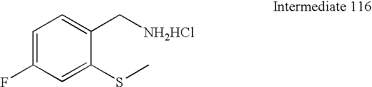

4-Fluoro-2-methylsulfanyl-benzylamine. 4-Fluoro-2-(methylthio)benzonitrile (prepared as in Anthony, N. J. et al. PCT Appl. WO 02/30931, 2002) (1.67 g, 0.1 mol) was dissolved in 20 mL THF and under N₂ treated with 10 mL 2M B₃.Me₂S. This was heated at 60° C. for 2 hrs. Heating was discontinued and 5 mL MeOH was cautiously added, followed by the cautious addition of 4 mL 6N HCl. Then 20 mL more H₂O added and EtOAc and the layers were separated. The aqueous layer was made basic with 1N NaOH and extracted with CH₂Cl₂. The extracts were dried (MgSO₄), filtered, concentrated and dried in vacuum to give intermediate 121 (1.3 g, 76%) as a solid. ¹H NMR (500 MHz, CDCl₃) δ: 7.20-7.31 (1H, m) 6.90 (1H, dd, J=2.4 Hz) 6.75-6.86 (1H, m) 3.86 (2H, s) 2.47 (3H, s). LC/MS (M+H): 172.

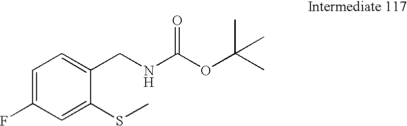

N-t-Butoxycarbonyl-(4-fluoro-2-(methylthio)phenyl)methanamine. A stirred solution of intermediate 121 (5.1 g, 0.03 mol) and 3.3 g triethylamine in 100 mL CH₂Cl₂ under N2 was treated with di-t-butyl dicarbonate (7.2 g, 0.033 mol) portionwise and stirred at room temperature for 30 min. Then, the reaction mixture was washed with dil HCl and water. The organic layer was dried over MgSO₄, filtered and concentrated to leave 8.1 g (100%) of intermediate 122 as a clear oil. ¹H NMR (500 MHz, CDCl₃) δ: 7.22-7.29 (1H, m) 6.89 (1H, dd, J=9.61, 2.29 Hz) 6.75-6.83 (1H, m) 4.93 (1H, s) 4.31 (2H, d, J=5.49 Hz) 2.47 (3H, s) 1.44 (9H, s). LC/MS (M+H): 272.

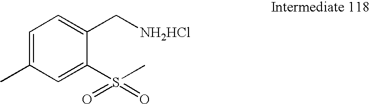

(4-Fluoro-2-(methylsulfonyl)phenyl)methanamine hydrochloride. A solution of intermediate 122 (8.1 g, 0.03 mol) in 100 mL acetone and 50 mL water was treated with oxone (18.5 g, 0.03 mol) and stirred for 10 min. Then an additional 18.5 g oxone was added and the mixture was warmed at 60° C. for 1.5 hrs. This was cooled, concentrated to remove acetone and extracted with CH₂Cl₂. This was concentrated to an oil, dissolved in 20 mL ethanol and treated with 10 mL 6N HCl and warmed at 60° C. for 2 h. Removal of solvents gave a gum which was crystallized from ethanol to give intermediate 143 (2.0 g) as crystals. The aqueous layer was made basic with ammonium hydroxide and extracted further with CH₂Cl₂ and concentration of the extracts gave a gum which was treated with HCl in ethanol to give an additional 0.9 g of intermediate 123. ¹H NMR (500 MHz, DMSO-D₆) δ: 8.54 (3H, s) 7.89 (1H, dd, J=8.54, 5.19 Hz) 7.67-7.85 (2H, m) 4.40 (2H, s) 3.41 (3H, s). LC/MS (M+H)=204.

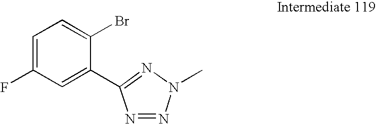

5-(2-Bromo-5-fluoro-phenyl)-2-methyl-2H-tetrazole: A mixture of 5-(2-bromo-5-fluoro-phenyl)-1H-tetrazole (1.0 g, 4.12 mmol; Butt Park Ltd.), methyl iodide (1.12 g, 10 mmol) and potassium carbonate (1.5 g) in DMF (5 mL) was stirred at room temperature for 16 hrs, and the mixture concentrated in vacuo. The residue was purified by column chromatography (SiO₂, CH₂Cl₂) to obtain 650 mg (2.53 mmol, Yield 61%) of the title compound as a white powder (fast-moving 2-Me isomer): TLC, Rf 0.7 (CHCl₂); ¹H NMR (500 MHz, CDCl₃) δ: 4.45 (3H, s) 7.03-7.11 (1H, m) 7.63 (1H, dd, J=8.9, 3.1 Hz) 7.69 (1H, dd, J=8.9, 5.5 Hz); ¹³C NMR (126 MHz, CDCl₃) δ: 39.86 (s) 116.28 (s) 118.66 (d, J=22 Hz) 118.76 (d, J=25 Hz) 130.13 (d, J=8.6 Hz) 135.73 (d, J=8.6 Hz) 161.74 (d, J=247.6 Hz) 163.53 (s); LC/MS m/z 257/259.

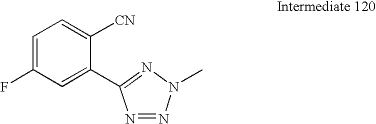

4-Fluoro-2-(2-methyl-2H-tetrazol-5-yl)-benzonitrile: A mixture of 5-(2-Bromo-5-fluoro-phenyl)-2-methyl-2H-tetrazole 124 (650 mg, 2.53 mmol) and CuCN (224 mg, 2.5 mmol) in DMF (4 mL) in a sealed tube was heated in an oil bath at 100-110° C. for 20 hrs. After cooling, the insoluble material was filtered, and the filtrate concentrated in vacuo. The residue dissolved in CH₂Cl₂ was washed with aq. 4N HCl and then with dil. NH₄OH, and then dried (MgSO₄), filtered, and concentrated. The residual solid was purified by column chromatography (SiO₂, CH₂Cl₂) to obtain 375 mg (1.85 mmol, Yield 73%) of the title compound as an off-white solid; 1H NMR (500 MHz, CDCl₃) δ: 4.48 (3H, s) 7.29 (1H, dd, J=7.6, 2.8 Hz) 7.85 (1H, dd, J=8.6, 5.2 Hz) 8.00 (1H, dd, J=9.0, 2.6 Hz); LC/MS m/z 204.

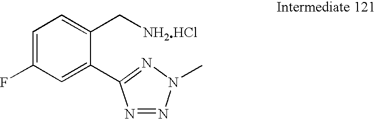

(4-Fluoro-2-(2-methyl-2H-tetrazol-5-yl)phenyl)methanamine hydrochloride: A solution of 4-fluoro-2-(2-methyl-2H-tetrazol-5-yl)-benzonitrile 125 (330 mg, 1.62 mmol) in EtOH (15 mL) was mixed with 6N HCl (1 mL) and 10% Pd—C (200 mg) under nitrogen, and the mixture hydrogenated with 1 atm of H₂ gas for 3 hrs. After removing the catalyst, the filtrate was concentrated in vacuo to complete dryness to obtain 360 mg (1.48 mmol, Yield 91%) of the title compound as an off-white solid; 1H NMR (500 MHz, DMSO-D6) δ: 4.42 (2H, d, J=2.75 Hz) 4.49 (3H, s) 7.48-7.56 (1H, m) 7.78 (1H, dd, J=8.7, 5.7 Hz) 7.86 (1H, dd, J=9.8, 2.8 Hz) 8.45 (3H, s); LC/MS m/z 208.

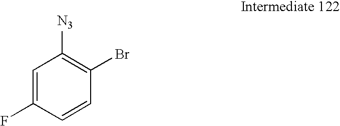

2-Azido-1-bromo-4-fluorobenzene: 2-Bromo-5-fluoro aniline (2.00 g, 10.53 mmol) was dissolved in concentrated HCl (10 mL) and water (10 mL) and cooled to 0 ⁰C. Aqueous NaNO₂ solution (1.090 g, 15.8 mmol of NaNO₂ in 10 mL of water) was added dropwise at such a rate that the temperature did not exceed 5 ⁰C. This mixture was stirred at 0 ⁰C. for 1.5 h. A solution of NaN₃ (1.027 g, 15.8 mmol) and NaOAc (12.95 g, 158 mmol) in water (50 mL) was then added at 0-5 ⁰C and the mixture was stirred for an additional 1 h at this temperature. The mixture was extracted with EtOAc and the combined extracts were washed with brine and dried over Na₂SO₄. The filtrate was concentrated to afford the title compound as a tan solid (2.188 g, 96%): ¹H NMR (400 MHz, CDCl₃) δ: 7.53 (1H, dd, J=8.8, 5.6 Hz), 6.94 (1H, dd, J=8.8, 2.8 Hz), 6.79 (1H, ddd, J=8.8, 7.6, 2.8 Hz).

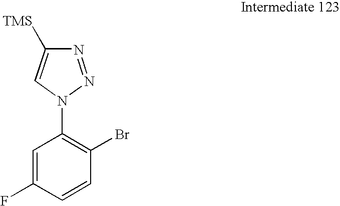

1-(2-Bromo-5-fluorophenyl)-4-(trimethylsilyl)-1H-1,2,3-triazole: A mixture of 2-azido-1-bromo-4-fluorobenzene (1.047 g, 4.85 mmol) and trimethylsilylacetylene (2.01 mL, 14.54 mmol) in toluene (5 mL) was heated in a pressure vessel at 110 ⁰C for 21.5 h. The reaction mixture was concentrated in vacuo and the residue was purified with a Biotage system on silica gel with hexanes:ethyl acetate (9:1) gradient as the eluent to afford the title compound as a colorless oil (1.451 g, 95% yield): ¹H NMR (400 MHz, CDCl₃) δ: 7.97 (1H, s), 7.74 (1H, dd, J=9.0, 5.4 Hz), 7.37 (1H, dd, J=8.5, 2.9 Hz), 7.16 (1H, ddd, J=8.8, 7.6, 3.0 Hz), 0.40 (9H, s), LCMS (⁺ESI, M+H⁺) m/z 314/316.

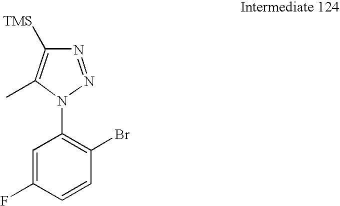

1-(2-Bromo-5-fluorophenyl)-5-methyl-4-(trimethylsilyl)-1H-1,2,3-triazole: The title compound was prepared according to an analogous procedure provided for 1-(2-bromo-5-fluorophenyl)-4-(trimethylsilyl)-1H-1,2,3-triazole. ¹H NMR (400 MHz, CDCl₃) δ: 7.73-7.69 (1H, m), 7.20-7.16 (2H, m), 2.22 (3H, s), 0.39 (9H, s); LCMS (⁺ESI, M+H⁺) m/z 328/330.

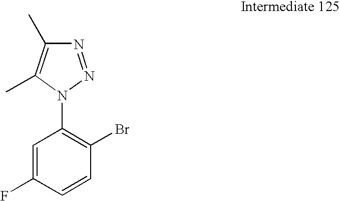

1-(2-bromo-5-fluorophenyl)-4,5-dimethyl-1H-1,2,3-triazole: The title compound was prepared according to an analogous procedure provided for 1-(2-bromo-5-fluorophenyl)-4-(trimethylsilyl)-1H-1,2,3-triazole ¹H NMR (400 MHz, CDCl₃) δ: 7.71 (1H, dd, J=8.8, 5.3 Hz), 7.12-7.20 (2H, m), 2.34 (3H, s), 2.12 (3H, s), LCMS (⁺ESI, M+H⁺) m/z 270/272.

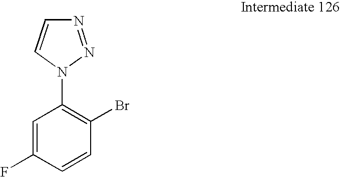

1-(2-Bromo-5-fluorophenyl)-1H-1,2,3-triazole: 1-(2-Bromo-5-fluorophenyl)-4-(trimethylsilyl)-1H-1,2,3-triazole (0.800 g, 2.55 mmol) was dissolved in THF (10 mL) and tetrabutylammonium fluoride (2.8 mL, 2.80 mmol, 1.0 M in THF) was added dropwise and the reaction mixture was stirred at 25 ⁰C for 4 h. The resulting mixture was concentrated in vacuo and the residue was purified with a Biotage system on silica gel with hexanes:ethyl acetate (8:2 to 7:3) gradient as the eluent to afford the title compound as a white solid (0.36 g, 58% yield): ¹H NMR (400 MHz, CDCl₃) δ: 8.06 (1H, d, J=1.0 Hz), 7.90 (1H, d, J=1.3 Hz), 7.76 (1H, dd, J=8.8, 5.3 Hz), 7.39 (1H, dd, J=8.3, 2.8 Hz), 7.19 (1H, ddd, J=8.9, 7.5, 3.0 Hz), LCMS (⁺ESI, M+H⁺) m/z 242/244.

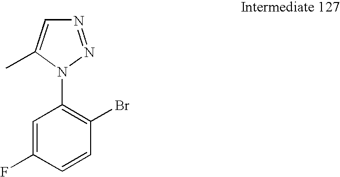

1-(2-Bromo-5-fluorophenyl)-5-methyl-1H-1,2,3-triazole. The title compound was prepared according to an analogous procedure provided for 1-(2-bromo-5-fluorophenyl)-1H-1,2,3-triazole. ¹H NMR (400 MHz, CDCl₃) δ: 7.76 (1H, dd, J=9.1, 5.3 Hz), 7.62 (1H, s), 7.19-7.26 (2H, m), 2.25 (3H, s), LCMS (⁺ESI, M+H⁺) m/z 256/258.

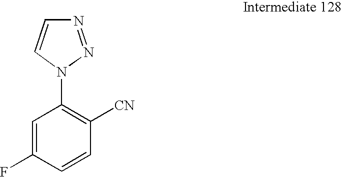

4-Fluoro-2-(1H-1,2,3-triazol-1-yl)benzonitrile: A mixture of 1-(2-bromo-5-fluorophenyl)-1H-1,2,3-triazole (0.603 g, 2.49 mmol), CuCN (0.245 g, 2.74 mmol), and 15 mL of NMP was subjected to microwave irradiation at 150 ⁰C for 0.5 h. The brown mixture was filtered over celite and washed with DMF. This solution was treated with 10% aqueous NH₄OH (28-30% solution) and extracted with EtOAc. The combined organic phase were successively washed with 10% aqueous NH₄OH (28-30% solution), saturated aqueous NH₄Cl, water, brine and dried over Na₂SO₄. The resulting mixture was concentrated in vacuo and the residue was purified with a Biotage system on silica gel with hexanes:ethyl acetate (7:3 to 6:4) gradient as the eluent to afford the title compound as a light yellow solid (0.285 g, 61% yield): ¹H NMR (400 MHz, CDCl₃) δ: 8.40 (1H, d, J=1.0 Hz), 7.96 (1H, s), 7.91 (1H, dd, J=8.6, 5.6 Hz), 7.77 (1H, dd, J=8.7, 2.4 Hz), 7.31-7.39 (1H, m).

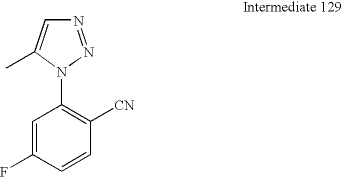

4-Fluoro-2-(5-methyl-1H-1,2,3-triazol-1-yl)benzonitrile. The title compound was prepared according to an analogous procedure provided for 4-fluoro-2-(1H-1,2,3-triazol-1-yl)benzonitrile. ¹H NMR (400 MHz, CDCl₃) δ: 7.93 (1H, dd, J=8.7, 5.4 Hz), 7.67 (1H, s), 7.44 (1H, ddd, J=8.6, 7.6, 2.5 Hz), 7.35 (1H, dd, J=8.1, 2.5 Hz), 2.39 (3H, s), LCMS (⁺ESI, M+H⁺) m/z 203.

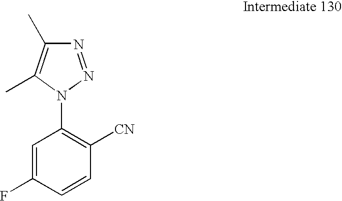

2-(4,5-dimethyl-1H-1,2,3-triazol-1-yl)-4-fluorobenzonitrile. The title compound was prepared according to an analogous procedure provided for 4-fluoro-2-(1H-1,2,3-triazol-1-yl)benzonitrile. ¹H NMR (400 MHz, CDCl₃) δ: 7.88 (1H, dd, J=8.7, 5.4 Hz), 7.38 (1H, ddd, J=8.7, 7.5, 2.5 Hz), 7.29 (1H, dd, J=8.1, 2.5 Hz), 2.37 (3H, s), 2.26 (3H, s), LCMS (⁺ESI, M+H⁺) m/z 217.

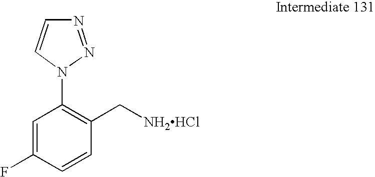

(4-Fluoro-2-(1H-1,2,3-triazol-1-yl)phenyl)methanamine hydrochloride: The title compound was prepared according to an analogous procedure provided for intermediate 126. ¹H NMR (400 MHz, DMSO-D6) δ: 8.73 (1H, d, J=1.0 Hz), 8.53 (3H, brs), 8.07 (1H, d, J=1.0 Hz), 7.91 (1H, dd, J=8.7, 5.9 Hz), 7.66 (1H, dd, J=9.2, 2.7 Hz), 7.60 (1H, td, J=8.5, 2.7 Hz), 3.92 (2H, q, J=5.6 Hz), LCMS (⁺ESI, M+H⁺) m/z 193.

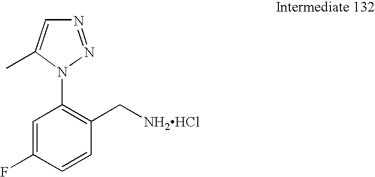

(4-Fluoro-2-(5-methyl-1H-1,2,3-triazol-1-yl)phenyl)methanamine hydrochloride: The title compound was prepared according to an analogous procedure provided for intermediate 126. ¹H NMR (400 MHz, DMSO-D6) δ: 8.61 (2H, s), 7.98 (1H, dd, J=9.5, 6.2 Hz), 7.80 (1H, s), 7.61-7.67 (2H, m), 3.66 (2H, q, J=5.7 Hz), 2.26 (3H, s), LCMS (⁺ESI, M+H⁺) m/z 206.

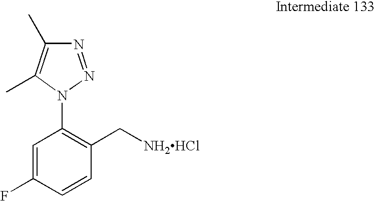

(2-(4,5-dimethyl-1H-1,2,3-triazol-1-yl)-4-fluorophenyl)methanamine hydrochloride: The title compound was prepared according to an analogous procedure provided for intermediate 126. ¹H NMR (400 MHz, DMSO-D6) δ: 8.58 (2H, s), 7.96 (1H, dd, J=8.7, 5.9 Hz), 7.56-7.65 (2H, n), 2.46-2.52 (2H, i), 2.30 (3H, s), 2.18 (3H, s), LCMS (⁺ESI, M+H⁺) m/z 221.

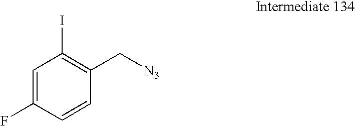

1-(Azidomethyl)-4-fluoro-2-iodobenzene: A solution of 1-(bromomethyl)-4-fluoro-2-iodobenzene (M. Protiva et al., Collect. Czech. Chem. Comm., 44, 1979, 2108-2123) (17.9 g, 56.8 mmol) in N,N-dimethylformamide (35 ml) was treated with sodium azide (5.0 g, 76.7 mmol) and the resulting mixture was heated to 50° C. for 4 h. The cooled mixture was filtered, the filtrate was concentrated in vacuo and the residue was chromatographed on silica gel (elution hexane) to give 15.7 g (97% yield) of the title azide as a clear oil. ¹HNMR 400 MHz (DMSO-d₆) δ: 4.53 (2H, s), 7.32 (1H, m), 7.54 (1H, dd, J=6.0, 8.6 Hz), 7.83 (1H, dd, J=3.0, 8.0 Hz).

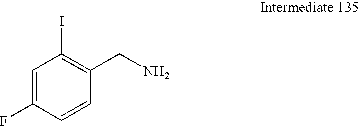

(4-Fluoro-2-iodophenyl)methanamine: A solution of 1-(azidomethyl)-4-fluoro-2-iodobenzene 139 (15.2 g, 54.8 mmol) in DMF (35 ml) at 0° C. was treated with triphenylphosphine (21.6 g, 81.2 mmol) and then stirred for 1 h. The reaction mixture was then treated with water (5 ml) and heated at 55° C. for 1 h. The DMF was concentrated in vacuo and the residue was diluted with ethyl acetate (200 ml). The organic phase was extracted with 0.5 N hydrochloric acid (140 ml) and the aqueous extract was washed with ethyl acetate. The aqueous phase was then adjusted to pH 9 with 1 N LiOH and extracted with ethyl acetate (2×200 ml). The combined organic phases were dried over anhydrous magnesium sulfate and concentrated. The residue was diluted with ether (200 ml), filtered and concentrated. Distillation of the residue in vacuo gave 8.52 g (62% yield) of the title amine as a clear oil: bp 85° C./0.35 torr (bulb to bulb distillation air bath temperature). ¹HNMR 400 MHz (DMSO-d₆) δ: 3.64 (2H, s), 7.27 (1H, m), 7.53 (1H, dd, J=6.0, 8.6 Hz), 7.83 (1H, dd, J=3.0, 8.0 Hz).

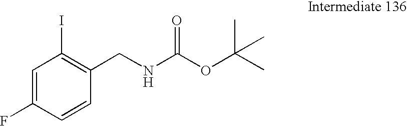

tert-Butyl 4-fluoro-2-iodobenzylcarbamate: A solution of (4-fluoro-2-iodophenyl)methanamine 140 (21.4 g, 85.2 mmol) in dichloromethane (350 ml) was treated at 0° C. with di-tert-butyl dicarbonate (20.5 g, 93.8 mmol) followed by triethylamine added drop wise over 30 min. The resulting mixture was then allowed to warm up to 25° C. and stirred for 18 h. The reaction mixture was then washed with water, brine, dried over anhydrous magnesium and concentrated. Chromatography of the residue on silica gel (elution gradient of ethyl acetate 5-20% in hexane) gave 28.37 g (95% yield) of the title carbamate as a clear oil. ¹HNMR 400 MHz (CDCl₃) δ: 1.47 (9H, s), 4.32 (2H, d, J=6.0 Hz), 5.04 (1H, broad), 7.07 (1H, m), 7.35 (1H, m), 7.56 (1H, dd, J=2.8, 8.0 Hz).

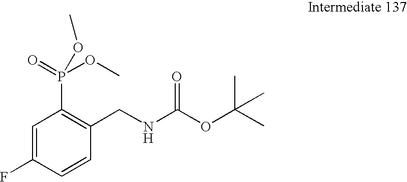

tert-Butyl) 2-(dimethoxyphosphoryl-4-fluorobenzylcarbamate). A solution of tert-butyl 4-fluoro-2-iodobenzylcarbamate 141 (5.00 g, 14.24 mmol), dimethyl phosphite (4.70 g, 42.7 mmol) and N,N-diisopropylethylamine (9.9 ml, 56.8 mmol) in methanol (75 ml) was flushed with argon and then treated with triphenylphosphine (0.5 g) and palladium(II) acetate (0.75 g). The resulting mixture was then sealed and heated at 100° C. for 1 hour. The cooled reaction mixture was concentrated in vacuo, diluted with ethyl acetate, washed with water, brine, dried over anhydrous magnesium and concentrated. Chromatography of the residue on silica gel (elution gradient of acetonitrile in dichloromethane) gave 3.24 g (68% yield) of the title phosphonate as a clear oil. ¹HNMR 400 MHz (CDCl₃) δ: 1.44 (9H, s), 3.81 (3H, s), 3.84 (3H, s), 4.49 (2H, d, J=6.0 Hz), 5.7 (1H, broad), 7.24 (1H, m), 7.47-7.7 (2H, m). HRMS (ESI⁺) calculated for C₁₄H₂₂FNO₅P [M+H⁺]: 334.1220. found: 334.1217.

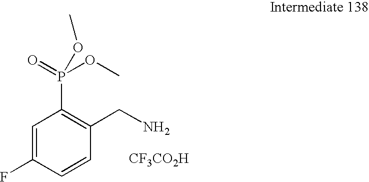

Dimethyl 2-(aminomethyl)-5-fluorophenylphosphonate trifluoroacetic acid salt: A solution of tert-butyl 2-(dimethoxyphosphoryl)-4-fluorobenzylcarbamate 142 (0.140 g, 0.42 mmol) in dichloromethane (5 ml) was treated with trifluoroacetic acid (5 ml) and the resulting mixture was stirred at 25° C. for 1 h. The solvent was then evaporated in vacuo to give the title amine salt as an amorphous white solid. MS (ESI⁺) m/z 234 [M+H⁺].

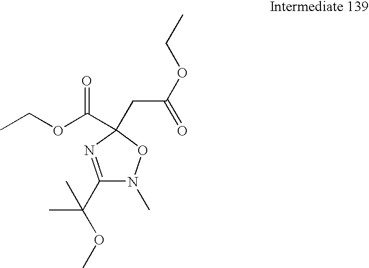

Ethyl 5-(2-ethoxyl-2-oxoethyl)-3-(2-methoxypropan-2-yl)-2-methyl-2,5-dihydro-1,2,4-oxadiazole-5-carboxylate: A round-bottom flask containing a mixture of 2,2-dimethoxypropane (2.08 g, 20 mmol) and ZnI₂ (5 mg) was placed in a water bath. To this mixture was added trimethylsilyl cyanide (2.7 mL, 20 mmol) via syringe and stirred overnight (16 h). To the resulting pale yellow solution was added EtOH (25 mL) and a solution of N-methylhydroxylamine hydrochloride (2.5, 30 mmol) and Na₂CO₃ (1.6 M, 15 mL) in water (25 mL). Then the reaction mixture was heated at 80° C. for 4 h and concentrated. The resulting residue was re-dissolved into 1:1 EtOH:H₂O (30 mL) and diethyl acetylenedicarboxylate (4.0 mL, 25 mmol) was added and stirred for 30 min. Then, diluted with ether (100 mL), washed with water (100 mL0, brine (20 mL), dried (Na₂SO₄), filtered and concentrated to give yellow oil. Flash chromatography with 3:1:1 Hex/Et₂O/CH₂Cl₂ provided product as a pale yellow oil (2.56 g, 41%). ¹H NMR (500 MHz, CDCl₃) δ: 4.33-4.18 (2H, m), 4.14 (2H, q, J=7.2 Hz), 3.33 (3H, s), 2.18 (1H, d, J=16.5 Hz), 3.24 (3H, s), 1.48 (3H, s), 1.47 (3H, s), 1.29 (3H, t, J=7.2 Hz), 1.24 (3H, t, J=7.2 Hz). HRMS (M+H) calcd for C₁₄H₂₄N₂O₆: 317.1713. found: 317.1702. Anal. calcd for C₁₄H₂₄N₂O₆: C, 53.15; H, 7.64; N, 8.85. found: C, 52.95; H, 7.51; N, 8.62.

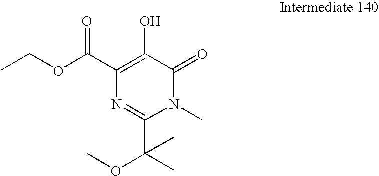

Ethyl 5-hydroxy-2-(2-methoxypropan-2-yl)-1-methyl-6-oxo-1,6-dihydropyrimidine-4-carboxylate: A solution of Intermediate 139 (3.6 g, 11.38 mmol) in xylenes (45 mL) was heated at reflux for 24 h. The resulting dark reaction cooled, diluted with ether (100 mL) and extracted with 0.2 M aq. Na₂CO₃ (4×25 mL). The combined aq. layers acidified with colic. HCl and extracted with CH₂Cl₂ (4×50 mL). The combined CH₂Cl₂ phases dried (Na₂SO₄), filtered and concentrated to give product as a brown solid (0.37 g, 12%). ¹H NMR (500 MHz, CDCl₃) δ: 10.42 (1H, s), 4.43 (2H, q, J=7.0 Hz), 3.86 (3H, s), 3.13 (3H, s), 1.61 (6H, s), 1.42 (3H, t, J=7.0 Hz). ¹³C NMR (125 MHz, CDCl₃) δ: 169.3, 159.8, 151.3, 148.5, 123.7, 80.1, 62.5, 51.5, 32.8, 26.0, 14.1. HRMS (M+H) calcd for C₁₂H₁₉N₂O₅: 271.1294. found: 271.1286. Anal. calcd for C₁₂H₁₈N₂O₅: C, 53.32; H, 6.71; N, 10.36. found: C, 53.04; H, 6.66; N, 10.53.

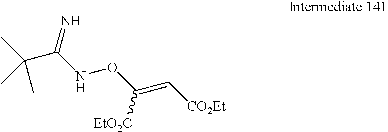

2-[(2,2-Dimethylpropanimidoyl)-aminooxy]-but-2-enedioic acid diethyl ester: Prepared according to the procedure for Intermediate 139. Yield: 99% crude, amber oil. ¹H NMR (500 MHz, DMSO-d₆) δ: 1.13 (˜⅔×9H, s), 1.18 (˜⅓×9H, s), 1.22 (˜⅓×3H, t, J=7 Hz), 1.24 (˜⅔×3H, t, J=7 Hz), 1.29 (˜⅔×3H, t, J=7 Hz), 1.32 (˜⅔×3H, t, J=7 Hz), 4.11 (˜⅓×2H, q, J=7 Hz), 4.14 (˜⅔×2H, q, J=7 Hz), 4.27 (˜⅔×2H, q, J=7 Hz), 4.31 (˜⅓×2H, q, J=7 Hz), 4.82 (2H, br s), 5.06 (2H, br s), 5.59 (⅔×1H, s), 5.78 (⅓×1H, s). ¹³C NMR (500 MHz, DMSO-d₆) δ: 13.97, 14.23, 14.31, 27.85, 27.91, 34.89, 35.30, 60.18, 60.36, 61.88, 62.21, 94.81, 101.18, 156.36, 161.07, 163.18, 163.31, 163.99, 164.08, 165.33, 166.66. HRMS (M+H) calcd for C₁₃H₂₃N₂O₅: 287.1607. found: 287.1609

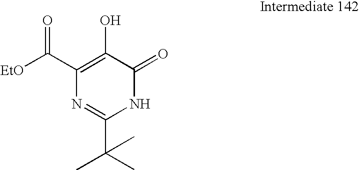

2-tert-Butyl-5-hydroxy-6-oxo-1,6-dihydro-pyrimidine-4-carboxylic acid ethyl ester: Prepared according to the procedure for Intermediate 140. Yield: 25%, light brown solid. ¹H NMR (CDCl₃, 500 MHz) δ: 1.39 (9H, s), 1.44 (3H, t, J=7 Hz), 4.45 (2H, q, J=7 Hz), 10.72 (1H, br s), 11.75 (1H, br s). ¹³C NMR (CDCl₃, 125.8 Hz) δ: 14.11, 28.34, 37.33, 62.79, 126.68, 149.05, 156.90, 160.00, 169.81. HRMS (M+H) calcd for C₁₁H₁₇N₂O₄: 241.1188. found: 241.1183

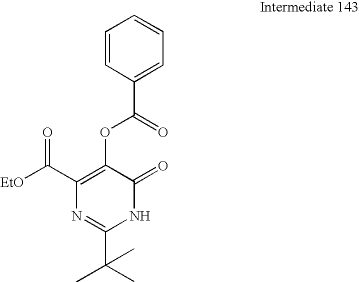

5-Benzoyloxy-2-tert-butyl-6-oxo-1,6-dihydro-pyrimidine-4-carboxylic acid ethyl ester: Yield: 74%, off-white powder. ¹H NMR (CDCl₃, 500 MHz): δ 1.15 (3H, t, J=7 Hz), 1.32 (9H, s), 4.28 (2H, q, J=7 Hz), 7.51 (2H, t, J=7.3 Hz), 7.65 (1H, t, J=7.3 Hz), 8.17 (2H, d, J=7.3 Hz), 12.51 (1H, br s). ¹³C NMR (CDCl₃, 125.8 Hz) δ: 13.97, 28.23, 37.93, 62.24, 128.54, 128.71, 130.51, 134.03, 136.68, 144.26, 160.31, 163.29, 163.50, 165.44. HRMS (M+H) calcd for C₁₈H₂₁N₂O₅: 345.1450. found: 345.1453

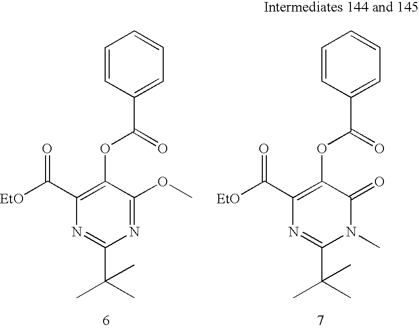

5-Benzoyloxy-2-tert-butyl-6-methoxy-pyrimidine-4-carboxylic acid ethyl ester (144) and 5-benzoyloxy-2-tert-butyl-1-methyl-6-oxo-1,6-dihydro-pyrimidine-4-carboxylic acid ethyl ester (145): To a solution of 5-benzoyloxy-2-tert-butyl-6-oxo-1,6-dihydro-pyrimidine-4-carboxylic acid ethyl ester (143) (1.03 g, 3.00 mmol) and methyl iodide (2.13 g, 15 mmol, 5 eq.) in THF (30 mL, Sure Seal, Aldrich) was added Cs₂CO₃ (1.17 g, 3.6 mmol; Aldrich) and the mixture stirred in an oil bath heated at 50° C. for 7.5 h. After cooling, the mixture diluted with EtOAc (30 mL) was washed with water (15 mL×2), brine (15 mL), dried (Na2SO4), filtered and concentrated in vacuo. The residual oil was purified by column chromatography (SiO2, 40 g; 40% EtOAc-Hexanes) to obtain 889 mg (2.48 mmol, Y. 83%) of O-methyl ether 144 as a clear oil: ¹H NMR (CDCl₃, 500 MHz) δ: 1.11 (3H, t, J=7 Hz), 1.41 (9H, s), 4.01 (3H, s), 4.26 (2H, q, J=7 Hz), 7.50 (2H, t, J=7.5 Hz), 7.64 (1H, t, J=7.5 Hz), 8.17 (2H, d, J=7.5 Hz); ¹³C NMR (CDCl₃, 125.8 Hz) δ: 13.93, 29.57, 39.67, 54.53, 62.08, 128.58, 128.76, 130.52, 130.64, 134.06, 147.50, 162.95, 163.59, 163.85, 174.13. HRMS (M+H) calcd for C₁₉H₂₃N₂O₅: 359.1607. found 359.1605. Further elution gave 132 mg (0.369 mmol, Y. 12%) of N-methyl ether 145 as a colorless oil: ¹H NMR (CDCl₃, 500 MHz) δ: 1.08 (3H, t, J=7 Hz), 1.47 (9H, s), 3.69 (3H, s), 4.22 (2H, q, J=7 Hz), 7.45 (2H, t, J=7.5 Hz), 7.59 (1H, t, J=7.5 Hz), 8.15 (2H, d, J=7.5 Hz). ¹³C NMR (CDCl₃, 125.8 Hz) δ: 13.92, 29.27, 34.43, 39.60, 62.00, 128.57, 128.65, 130.60, 133.99, 136.00, 140.50, 160.00, 163.27, 163.69, 163.83. HRMS (M+H) calcd for C₁₉H₂₃N₂O₅: 359.1607. found 359.1591

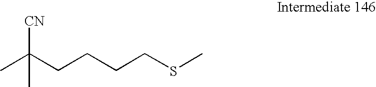

2,2-Dimethyl-6-methylsulfanyl-hexanenitrile: To a solution of 6-bromo-2,2-dimethyl-hexanenitrile (5.10 g, 25 mmol; Aldrich) in abs. EtOH (25 mL) was added NaSMe (2.10 g, 30 mmol; Aldrich) and the mixture stirred at room temperature under nitrogen atmosphere for 18 h. The insoluble materials were filtered and the filtrate extracted with Et₂O (50 mL). The ether extract was washed with water twice, brine, dried (Na₂SO₄), filtered and concentrated in vacuo to obtain 4.15 g (24.3 mmol, Y. 97%) of the Intermediate 146 as a clear amber oil. ¹H NMR (CDCl₃, 500 MHz) δ: 1.31 (6H, s), 1.51 (2H, m), 1.54 (2H, m), 1.60 (2H, m), 2.07 (3H, s), 2.48 (2H, t, J=7 Hz). ¹³C NMR (CDCl₃, 125.8 Hz) δ: 15.62, 24.53, 26.72, 29.14, 32.42, 33.99, 40.73, 125.12. HRMS (M+H) calcd for C₉H₁₇NS; 172.1160. found: 172.1160.

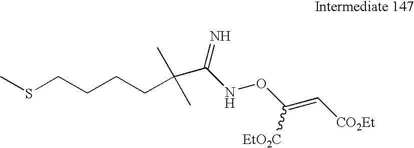

2-[2,2-Dimethyl-6-methylsulfanyl-hexanimidoyl)-aminooxy]-but-2-enedioic acid diethyl ester: Prepared according to the procedure for Intermediate 139 using Intermediate 146. Yield: 63% as a ˜1:1 mixture of isomer, amber colored clear oil. ¹H NMR (CDCl₃, 500 MHz) δ: Isomer A; 1.10 (6H, s), 1.25 (3H, t, J=7.2 Hz), 1.31 (3H, t, J=7.2 Hz), 1.34 (2H, m), 1.42 (2H, m), 1.53 (2H, qt, J=7.3 Hz), 2.06 (3H, s), 2.45 (2H, t, J=7.5 Hz), 4.15 (2H, q, J=7.2 Hz), 4.27 (2H, q, J=7.2 Hz), 5.05 (2H, br s), 5.58 (1H, s). Isomer B; 1.14 (6H, s), 1.21 (3H, t, J=7.2 Hz), 1.32 (3H, t, J=7.2 Hz), ˜1.3 (2H, m), ˜1.42 (2H, m), 1.53 (2H, qt, J=7.3 Hz), 2.04 (3H, s), 2.44 (2H, t, J=7.3 Hz), 4.11 (2H, q, J=7 Hz), 4.30 (2H, q, J=7.2 Hz), 4.79 (2H, br s), 5.76 (1H, s). ¹³C NMR (CDCl₃, 125.8 Hz) δ: Isomer A; 14.26, 14.34, 15.61, 23.73, 25.63, 29.71, 34.10, 38.09, 40.97, 60.32, 61.84, 101.02, 156.66, 162.74, 163.16, 165.24. Isomer B; 14.10, 14.34, 15.59, 23.77, 25.75, 29.59, 34.07, 38.52, 40.92, 60.15, 62.18, 94.87, 160.02, 162.78, 163.26, 166.61. HRMS (M+H) calcd for C₁₇H₃₁N₂O₅S: 375.1954. found: 375.1970

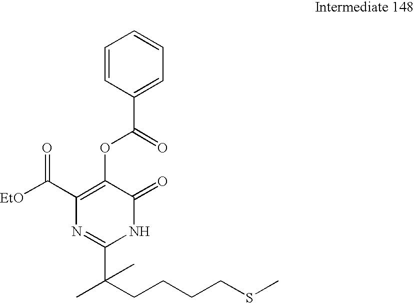

Ethyl 5-(benzoyloxy)-2-(2-methyl-6-(methylthio)hexan-2-yl)-6-oxo-1,6-dihydropyrimidine-4-carboxylate: Prepared according to the procedure for Intermediate 143. Yield: 35%, amber colored clear oil. ¹H NMR (CDCl₃, 500 MHz) δ; 1.14 (3H, t, J=7 Hz), 1.25 (2H, n), 1.33 (6H, s), 1.50 (2H, qt. J=7.3 Hz), 1.69 (2H, m), 2.03 (3H, s), 2.40 (2H, t, J=7.3 Hz), 4.28 (2H, q, J=7 Hz), 7.51 (2H, t, J=7.5 Hz), 7.65 (1H, J=7.5 Hz), 8.17 (2H, d, J=7.5 Hz), 12.5 (1H, br s). ¹³C NMR (CDCl₃, 125.8 Hz) δ: 13.96, 15.51, 23.72, 26.04, 29.35, 33.90, 40.73, 41.21, 62.23, 128.48, 128.74, 130.19, 130.54, 133.55, 134.07, 136.59, 144.26, 160.55, 163.18, 163.45, 164.72, 171.13. HRMS (M+H) calcd for C₂₂H₂₉N₂O₅S: 433.1797. found: 433.1800

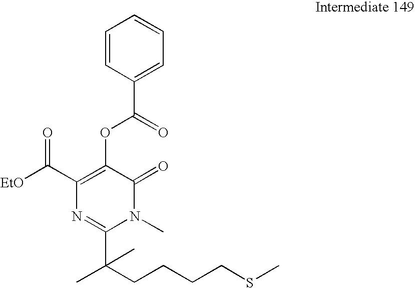

Ethyl-5-(benzoyloxy)-1-methyl-2-(2-methyl-6-(methylthio)hexan-2-yl)-6-oxo-1,6-dihydro-pyrimidine-4-carboxylate: Prepared according to the procedure for intermediate 145. Yield: 4.5%, colorless oil. ¹H NMR (CDCl₃, 500 MHz) δ: 1.12 (3H, t, J=7.2 Hz), 1.33 (2H, m), 1.49 (6H, s), 1.59 (2H, qt, J=7.5 Hz), 1.84 (2H, m), 2.07 (3H, s), 2.47 (2H, t, J=7.3 Hz), 3.71 (3H, s), 4.26 (2H, q, J=7 Hz), 7.49 (2H, t, J=7.7 Hz), 7.63 (1H, t, J=7.5 Hz), 8.18 (2H, d, J=7 Hz). ¹³C NMR (CDCl₃, 125.8 Hz) δ: 13.97, 15.63, 24.11, 28.04, 29.57, 33.96, 34.00, 40.99, 43.31, 62.05, 128.60, 128.68, 130.64, 134.00, 136.09, 140.30, 160.06, 162.85, 163.23, 163.72. HRMS (M+H) calcd for C₂₃H₃₁N₂O₅S: 447.1954. found: 447.1955

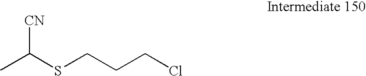

2-(3-Chloropropylthio)propanenitrile: To a stirred solution of 2-chloropropionitrile (8.953 g, 100 mmol) and 3-chloro-1-propanethiol (10 g, 90 mmol) in acetonitrile (100 mL) was added K₂CO₃ (13.821 g, 100 mmol). After stirring 24 h at room temperature, the reaction mixture was warmed to 60° C. and stirred additional 24 h. Then, the reaction mixture was cooled, diluted with CH₂Cl₂ (100 mL), filtered and concentrated to give light brown liquid which was used in the next step without purification.

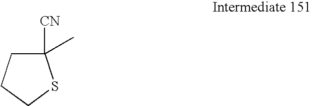

2-Methyl-tetrahydrothiophene-2-carbonitrile: To a stirred 1M solution of LiHMDS (150 mL) in THF was rapidly added via cannula a solution of Intermediate 150 (14.9 g) in THF (50 mL) over 10 minutes at room temperature. The addition flask was rinsed with THF (20 mL) and added to the reaction mixture. The resulting dark reaction mixture was stirred for 16 h, quenched with sat. NH4Cl (1 ml), diluted with CH₂Cl₂ (100 mL), dried (Na₂SO₄), filtered and concentrated to give dark liquid. Distilled to provide product as light yellow liquid (8.34 g, 73%, bp: ˜80-85° C. @ 3-4 mmHg). ¹H NMR (500 MHz, CDCl₃) δ: 3.17-3.08 (2H, m), 2.50-2.46 (1H, m), 2.32-2.22 (2H, m), 1.90-1.84 (1H, m), 1.75 (3H, s).

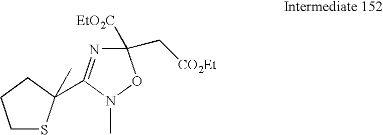

Ethyl 5-(2-ethoxy-2-oxoethyl)-2-methyl-3-(2-methyl-tetrahydrothiophen-2-yl)-2,5-dihydro-1,2,4-oxadiazole-5-carboxylate: Prepared according to the procedure for Intermediate 139 using Intermediate 151: Yield: 34%, yellow oil. ¹H NMR (500 MHz, CDCl₃) δ: 4.35-4.11 (4H, m), 3.29 (0.5H, s), 3.27 (1.5H, s), 3.26 (0.45H, s), 3.25 (1.5H, s), 3.06-2.88 (3H, m), 2.79-2.73 (1H, m), 2.25-2.18 (1H, m), 2.14-2.06 (1H, m), 1.85-1.79 (1H, m), 1.63 (1.5H, s), 1.62 (1.5H, s), 1.31-1.23 (6H, m). HRMS (M+H) calcd for C₁₅H₂₅N₂O₄S: 345.1484. found: 345.1493.

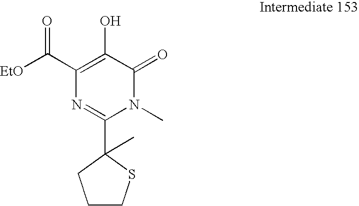

Ethyl 5-hydroxy-1-methyl-2-(2-methyl-tetrahydrothiophen-2-yl)-6-oxo-1,6-dihydro-pyrimidine-4-carboxylate: Prepared according to the procedure for Intermediate 140. Yield: 11% crude, brown solid and used without purification. LCMS (M+H) calcd for C₁₃H₁₉N₂O₄S: 299.36. found: 299.26.

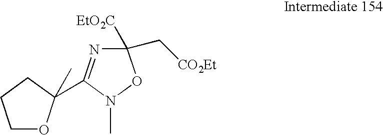

5-Ethoxycarbonylmethyl-2-methyl-3-(2-methyl-tetrahydro-furan-2-yl)-2,5-dihydro-[1,2,4]oxadiazole-5-carboxylic acid ethyl ester: Prepared according to the procedure for Intermediate 139 using 2-methyl-tetrahydro-furan-2-carbonitrile (This compound was prepared by following the procedure described in Y. Levoux, Bull. Chim. Soc., France 1968, 344). Yield: 64% as a mixture of diastereomers, an amber colored oil. ¹H NMR (CDCl₃, 500 MHz) δ: 1.23 (3H, t, J=7.2 Hz), 1.28 (3H, t, J=7.0 Hz), 1.49, 1.51 (3H, 2s), 1.74-1.82 (1H, m), 1.88-1.99 (2H, m), 2.56-2.64 (1H, m), 2.91 (1H, dd, 1-16.2, 13.1 Hz), 3.24 (1H, dd, J=16.2, 13.1 Hz), 3.30 (1.5H, s), 3.31 (1.5H, s) 3.79-3.86 (1H, m), 3.86-3.95 (1H, m), 4.09-4.15 (2H, m), 4.16-4.24 (1H, m), 4.24-4.32 (1H, m). ¹³C NMR (CDCl₃, 125.8 Hz) δ: 14.1, 14.2, 25.3, 25.4, 25.8, 26.0, 38.7, 38.8, 41.7, 41.8, 42.1, 42.2, 60.8, 60.8, 62.0, 62.1, 68.6, 68.6, 81.1, 81.1, 103.4, 103.7, 168.5, 168.6, 168.8, 168.9, 170.4, 170.6. HRMS (M+H) calcd for C₁₅H₂₅N₂O₆: 329.1713. found: 329.1701.

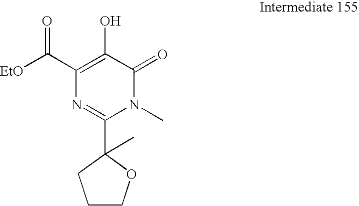

5-Hydroxy-1-methyl-2-(2-methyl-tetrahydro-furan-2-yl)-6-oxo-1,6-dihydro-pyrimidine-4-carboxylic acid ethyl ester: Prepared according to the procedure for Intermediate 140. Yield: 21%, beige solid. ¹H NMR (CDCl₃, 500 MHz) δ: 1.42 (3H, t, J=7.2 Hz), 1.58 (3H, s), 1.76-1.84 (1H, m), 1.84-1.91 (1H, m), 1.91-2.01 (1H, m) 3.22-3.30 (1H, m), 3.69-3.76 (1H, m), 3.81 (3H, s), 3.95-4.02 (1H, m), 4.36-4.50 (2H, m), 10.37 (1H, s). ¹³C NMR (CDCl₃, 125.8 Hz) δ: 14.2, 25.0, 25.7, 33.7, 38.1, 62.5, 68.8, 85.7, 123.7, 148.3, 151.8, 159.9, 169.3. HRMS (M+H) calcd for C₁₄H₁₉N₂O₅: 283.1294. found: 283.1285.

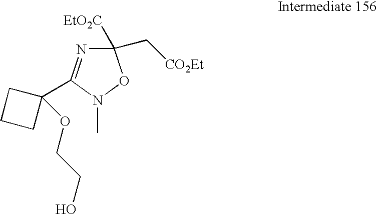

2-{[2-Hydroxy-ethoxy)-cyclobutanecarboximidoyl]-aminooxy}-but-2-enedioic acid diethyl ester: Prepared according to the procedure for Intermediate 139 using 1-(2-trimethylsilanyloxy-ethoxy)-cyclobutanecarbonitrile (This compound was prepared following the procedure by B. N. Naidu, PCT Appl. WO 200510, 2005). Yield: 54%, amber colored oil. ¹H NMR (CDCl₃, 500 MHz) δ: 1.22 (3H, t, J=7 Hz), 1.27 (3H, t, J=7 Hz), 1.75-1.8 (2H, m), 2.20-2.28 (2H, m), 2.52-2.56 (2H, m), 2.96-3.26 (2H, m), 3.43-3.45 (2H, m), 3.68 (2H, br s), 4.12 (2H, q, J=7 Hz), 4.17-4.30 (2H, m). ¹³C NMR (CDCl3, 125.8 Hz) δ: 13.16, 14.09, 14.21, 31.79, 40.56, 41.77, 60.84, 62.18, 61.40, 65.91, 76.34, 104.14, 167.08, 168.38, 168.79. HRMS (M+H) calcd for C₁₆H₂₇N₂O₇: 359.1818. found: 359.1814.

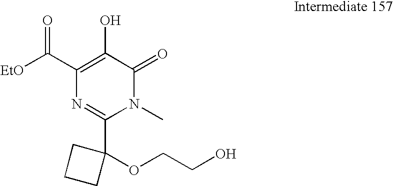

5-Hydroxy-2-[1-(2-hydroxy-ethoxy)-cyclobutyl]-1-methyl-6-oxo-1,6-dihydro-pyrimidine-4-carboxylic acid ethyl ester: Prepared according to the procedure for Intermediate 140. Yield, 7.8%, amber colored oil. ¹H NMR (CDCl₃, 500 MHz) δ: 1.40 (3H, t, J=7 Hz), 1.68 (1H, m), 1.92 (1H, m), 2.34 (2H, dd, J=11, 9.5 Hz), 2.80 (2H, t, J=12 Hz), 3.22 (2H, t, J=4.5 Hz), 3.58 (3H, s), 3.65 (2H, t, J=4.5 Hz), 4.42 (2H, q, J=7 Hz), 10.51 (1H, br s). ¹³C NMR (CDCl₃, 125.8 Hz) δ; 12.92, 14.13, 31.42, 32.32, 61.77, 62.57, 65.23, 81.68, 123.80, 148.82, 149.05, 159.50, 169.41. HRMS (M+H) calcd for C₁₄H₂₁N₂O₆; 313.1400. found: 313.1392.

### EXAMPLE 1

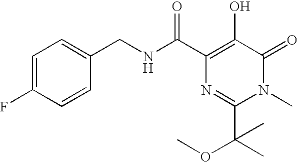

N-(4-Fluorobenzyl)-5-hydroxy-2-(2-methoxypropan-2-yl)-1-methyl-6-oxo-1,6-dihydropyrimidine-4-carboxamide: A solution of Intermediate 140 (0.072 g, 0.2664 mmol), 4-fluorobenzylamine (0.10 g, 8 mmol) and Et₃N (0.1 ml, 0.714 mmol) in DMF/EtOH (1:1, 3 mL) was heated in a sealed vial for 2 h. Then, the reaction mixture was collected and purified by prep-HPLC to afford product as white solid (0.515 g, 55%). ¹H NMR (500 MHz, CDCl₃) δ: 11.93 (1H, s), 7.74 (1H, br s), 7.33-7.30 (2H, m), 7.05 (2H, t, J=8.5 Hz), 4.58 (2H, d, J=6.4 Hz), 3.85 (3H, s), 3.11 (3H, s), 1.57 (6H, s). HRMS (M+H) calcd for C₁₇H₂₁FN₃O₄: 350.1516. found: 350.1517. Anal. calcd for C₁₇H₂₀FN₃O₄.0.25MeOH: C, 57.85; H, 5.83; N, 11.91; F, 5.38. found: C, 57.61; H, 5.89; N, 11.60; F, 5.45.

### EXAMPLE 2

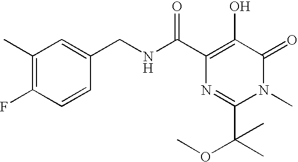

N-(4-Fluoro-3-methylbenzyl-5-hydroxy-2-(2-methoxypropan-2-yl)-1-methyl-6-oxo-1,6-dihydropyrimidine-4-carboxamide: Yield: 0.616 g, 64%; white solid. ¹H NMR (500 MHz, CDCl₃) δ: 11.97 (1H, s), 7.72 (1H, br s), 7.16-7.09 (2H, m), 6.98 (1H, t, J=8.5 Hz), 4.54 (2H, d, J=6.1 Hz), 3.85 (3H, s), 3.12 (3H, s), 2.27 (3H, s), 1.58 (6H, s). HRMS (M+H) calcd for C₁₈H₂₃FN₃O₄: 364.1673. found: 364.1671. Anal. calcd for C₁₈H₂₂FN₃O₄: C, 59.49; H, 6.10; N, 11.56; F, 5.22. found: C, 59.59; H, 6.00; N, 11.52; F, 5.47.

### EXAMPLE 3

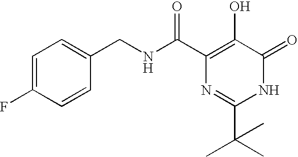

2-tert-Butyl-5-hydroxy-6-oxo-1,6-dihydro-pyrimidine-4-carboxylic acid 4-fluoro-benzylamide: Yield: 46%, off-white crystalline solid. ¹H NMR (CDCl₃, 500 MHz) δ: 1.35 (9H, s, 8.9), 4.58 (2H, d, J=6 Hz), 7.05 (2H, t, J=8.5 Hz), 7.32 (2H, dd, J=8.5, 5.5 Hz), 7.97 (1H, t, J=6 Hz), 11.13 (1H, br s), 12.08 (1H, s). ¹³C NMR (CDCl₃, 125.8 Hz) δ: 29.00, 37.63, 43.09, 116.41, 116.57, 127.52, 130.03, 130.09, 133.79, 148.07, 157.23, 160.28, 162.09, 164.04, 169.17. HRMS (M+H) calcd for C₁₆H₁₉FN₃O₃: 320.1410. found 320.1407. Anal. Calcd for C₁₆H₁₈FN₃O₃: C, 60.18; H, 5.68; N, 13.15. found: C, 60.18; H, 5.80; N, 13.10.

### EXAMPLE 4

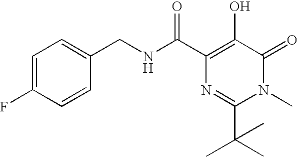

2-tert-Butyl-5-hydroxy-1-methyl-6-oxo-1,6-dihydro-pyrimidine-4-carboxylic acid 4-fluoro-benzylamide: Yield: 43%, white crystals. ¹H NMR (CDCl₃, 500 MHz) δ: 1.43 (9H, s), 3.69 (3H, s), 4.57 (2H, d, J=6.5 Hz), 7.03 (2H, t, J=8.5 Hz), 7.30 (2H, dd, J=8.5, 5.3 Hz), 7.86 (1H, brs), 11.74 (1H, s). ¹³C NMR (CDCl₃, 125.8 Hz) δ: 29.43, 38.76, 38.76, 42.39, 115.76, 115.93, 124.24, 129.34, 129.40, 133.29, 133.32, 146.21, 155.92, 160.11, 161.43, 163.39, 168.66. HRMS (M+H) calcd for C₁₇H₂₁FN₃O₃: 334.1567. found: 334.1561. Anal, Calcd for C₁₇H₂₀FN₃O₃.0.9EtOH.0.2H₂O: C, 59.67; H, 6.87; N, 11.10. found: C, 59.44; H, 6.99; N, 11.29.

### EXAMPLE 5

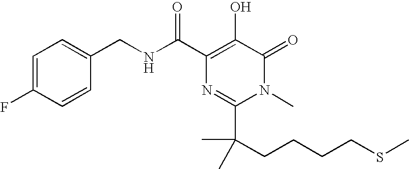

N-(4-Fluorobenzyl)-5-hydroxy-1-methyl-6-methylthio)hexan-2-yl)-6-oxo-1,6-dihydropyrimidine-4-carboxamide: Yield: 32%, white crystalline powder. ¹H NMR (CDCl₃, 500 MHz) δ: 1.21 (2H, m), 1.41 (6H, s), 1.53 (2H, qt. J=7.3 Hz), 1.78 (2H, m), 2.04 (3H, s), 2.43 (2H, t, J=7.3 Hz), 3.69 (3H, s), 4.58 (2H, d, J=6 Hz), 7.06 (2H, t, J=8.5 Hz), 7.72 (2H, dd, J=8.5, 5.5 Hz), 7.83 (1H, t, J=6 Hz), 11.76 (1H, s). ¹³C NMR (CDCl₃, 125.8 Hz) δ: 15.65, 23.98, 28.45, 29.40, 33.76, 33.97, 40.72, 42.48, 115.82, 115.99, 124.22, 129.41, 129.48, 133.25, 133.27, 146.22, 154.87, 160.05, 161.48, 163.44 68.63. HRMS (M+H) calcd for C₂₁H₂₉FN₃O₃S: 422.1914. found: 422.1898

### EXAMPLE 6

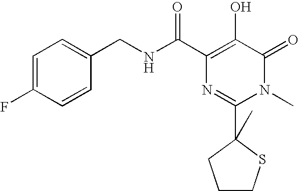

N-(4-Fluorobenzyl)-5-hydroxy-1-methyl-2-(2-methyl-tetrahydrothiophen-2-yl)-6-oxo-1,6-dihydropyrimidine-4-carboxamide: Yield: 0.1113 g, 42%; white solid. ¹H NMR (500 MHz, CDCl₃) δ: 11.85 (1H, s), 7.69 (1H, br s), 7.31-7.28 (2H, m), 7.05 (2H, t, J=8.5 Hz), 4.62-4.54 (2H, m), 3.67 (3H, s), 3.10-3.05 (1H, m), 2.99-2.90 (2H, m), 2.13-2.08 (2H, m), 1.96-1.91 (1H, m), 1.75 (3H, s). HRMS (M+H) calcd for C₁₈H₂₁FN₃O₃S: 378.1288. found: 378.1269. Anal. calcd for C₁₈H₂₀FN₃O₃S: C, 57.28; H, 5.34; N, 11.13. found: C, 57.28; H, 5.41; N, 11.13.

### EXAMPLE 7

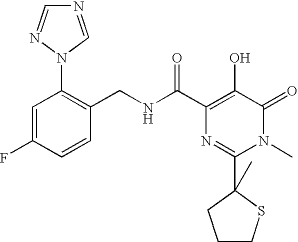

N-(4-Fluoro-2-(1H-1,2,4-triazol-1-yl)benzyl)-5-hydroxy-1-methyl-2-(2-methyl-tetrahydrothiophen-2-yl)-6-oxo-1,6-dihydropyrimidine-4-carboxamide: Yield: 0.0938 g, 71%; purple powder. ¹H NMR (500 MHz, CDCl₃) δ: 11.91 (1H, s), 8.80 (1H, t, J=6.4 Hz), 8.45 (1H, s), 8.16 (1H, s), 7.72 (1-H, dd, J=8.5, 5.8 Hz), 7.21 (1H, td, J=8.2, 2.4 Hz), 7.11 (1H, dd, J=8.2, 2.4 Hz), 4.44 (2H, d, J=7.0 Hz), 3.66 (3H, s), 3.24-3.19 (1H, m), 3.11-3.07 (1H, m), 2.99-2.94 (1H, m), 2.19-2.13 (2H, m), 2.02-1.97 (1H, m), 1.76 (3H, s). HRMS (M+H) calcd for C₂₀H₂₂FN₆O₃S: 445.1458. found: 445.1470. Anal. calcd for C₂₀H₂₁FN₆O₃S: C, 54.04; H, 4.76; N, 18.90. found: C, 53.86; H, 4.55; N, 18.67.

### EXAMPLE 8

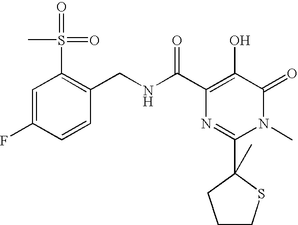

N-(4-Fluoro-2-(methylsulfonyl)benzyl)-5-hydroxy-1-methyl-2-(2-methyl-tetrahydrofuran-2-yl)-6-oxo-1,6-dihydropyrimidine-4-carboxamide: Yield: 0.0981 g, 73%; white solid. ¹H NMR (500 MHz, CDCl₃) δ: 11.67 (1H, s), 8.54 (1H, t, J=6.4 Hz), 7.75 (1H, dd, J=7.9, 2.7 Hz), 7.72 (1H, dd, J=8.5, 5.2 Hz), 7.34 (1H, td, J=8.2, 2.7 Hz), 4.83-4.76 (2H, m), 3.64 (3H, s), 3.17 (3H, s), 3.15-3.03 (2H, m), 2.96-2.91 (1H, m), 2.21-2.05 (2H, m), 1.95-1.90 (1H, m), 1.73 (3H, s). HRMS (M+H) calcd for C₁₉H₂₃FN₃O₅S₂: 456.1063. found: 456.1073. Anal. calcd for C₁₉H₂₂FN₃O₅S₂: C, 50.09; H, 4.86; N, 9.22. found: C, 50.01; H, 4.59; N, 9.05.

### EXAMPLE 9

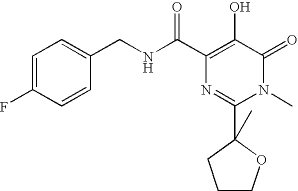

N-(4-Fluorobenzyl-5-hydroxy-1-methyl-2-(2-methyl-tetrahydrofuran-2-yl)-6-oxo-1,6-dihydropyrimidine-4-carboxamide: Yield: 48%, pinkish crystals. ¹H NMR (CDCl₃, 500 MHz) δ: 1.58 (3H, s) 1.83-1.94 (2H, m), 1.95-2.07 (1H, m), 2.91-3.01 (1H, m), 3.78 (1H, q, J=7.3 Hz), 3.83 (3H, s), 4.00-4.08 (1H, m), 4.56-4.67 (2H, m), 7.08 (2H, t, J=8.5 Hz), 7.34 (2H, dd, J=8.2, 5.5 Hz), 7.77 (1H, br s), 11.93 (1H, s). ¹³C NMR (CDCl₃, 125.8 Hz) δ: 24.8, 25.8, 33.7, 38.4, 42.4, 68.7, 85.3, 115.78, 115.95, 124.3, 129.39, 129.46, 133.24, 133.27, 147.1, 152.2, 159.8, 161.47, 163.43, 168.5. HRMS (M+H) calcd for C₁₈H₂₁FN₃O₄: 362.1516. found: 362.1526. Anal. calcd for C₁₈H₂₀FN₃O₄: C, 59.82; H, 5.57; N, 11.62. found: C, 59.64; H, 5.30; N, 11.67.

### EXAMPLE 10

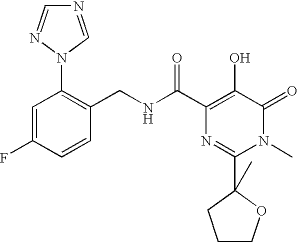

N-(4-Fluoro-2-(1H-1,2,4-triazol-1-yl)benzyl)-5-hydroxy-1-methyl-2-(2-methyl-tetrahydrofuran-2-yl)-6-oxo-1,6-dihydropyrimidine-4-carboxyamide: Yield: 59%, beige crystalline solid. ¹H NMR (CDCl₃, 500 MHz) δ: 1.57 (3H, s), 1.83-1.95 (2H, m), 1.95-2.05 (1H, m), 3.17-3.25 (1H, m), 3.69-3.77 (1H, m), 3.79 (3H, s) 3.96-4.04 (1H, m), 4.38-4.50 (2H, m) 7.11 (1H, dd, J=8.4, 2.6 Hz), 7.17-7.23 (1H, m), 7.70 (1H, dd, J=8.5, 6.1 Hz), 8.16 (1H, s), 8.47 (1H, s), 8.82 (1H, t, =6.4 Hz), 11.88 (1H, s). ¹³C NMR (CDCl₃, 125.8 Hz) δ: 24.8, 25.7, 35.5, 38.2, 39.1, 68.8, 85.5, 112.2, 112.4, 116.9, 117.1, 124.3, 128.67, 128.70, 134.37, 134.44, 136.89, 136.96, 144.0, 147.0, 151.7, 152.9, 159.9, 161.2, 163.2, 168.4. HRMS (M+H) calcd for C₂₀H₂₂FN₆O₄: 429.1687. found: 429.1677. Anal. calcd for C₂₀H₂₁N₆O₄: C, 56.07; H, 4.94; N, 19.61. found: C, 56.06; H, 4.71; N, 19.39.

### EXAMPLE 11

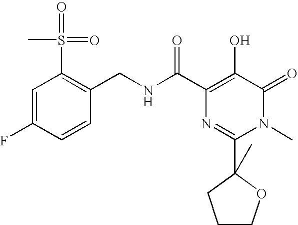

N-(4-Fluoro-2-(methylsulfonyl)benzyl)-5-hydroxy-1-methyl-2-(2-methyl-tetrahydrofuran-2-yl)-6-oxo-1,6-dihydropyrimidine-4-carboxyamide: Yield: 60%, off-white crystals. ¹H NMR (CDCl₃, 500 MHz) δ: 1.54 (3H, s) 1.81-1.92 (2H, m), 1.92-2.04 (1H, m), 3.02-3.12 (1H, m), 3.18 (3H, s), 3.67-3.75 (1H, m), 3.78 (3H, s), 3.94-4.03 (1H, m), 4.81 (2H, d, J=6.7 Hz), 7.34 (1H, dt, J=8 Hz, 3 Hz), 7.72 (H, dd, J=8 Hz, 5 Hz), 7.75 (1H, dd, J=8, 3 Hz), 8.55 (1H, t, J=6.4 Hz), 11.69 (1H, S). ¹³C NMR (CDCl₃, 125.8 Hz) δ: 24.9, 25.7, 33.6, 38.2, 40.2, 45.1, 68.8, 85.4, 117.39, 117.59, 121.52, 121.69, 24.2, 132.88, 132.91, 135.24, 135.30, 140.69, 140.74, 146.9, 152.1, 159.8, 161.10, 163.12, 168.3. HRMS (M+H) calcd for C₁₉H₂₃FN₃O₆S: 440.1292. found: 440.1292. Anal. calcd for C₁₉H₂₂FN₃O₆S: C, 51.92; H, 5.04; N, 9.56. found C, 51.64; H, 4.95; N, 9.33.

### EXAMPLE 12

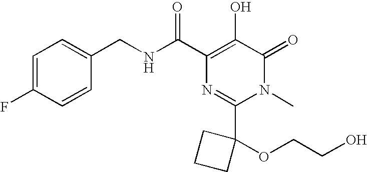

5-Hydroxy-2-[1-(2-hydroxy-ethoxy)-cyclobutyl]-1-methyl-6-oxo-1,6-dihydro-pyrimidine-4-carboxylic acid 4-fluoro-benzylamide: Yield: 48%, white crystalline powder. ¹H NMR (CDCl₃, 500 MHz) δ: 1.68 (1H, m), 1.96 (1H, m), 2.38 (2H, dd, J=11, 9.5 Hz), 2.69 (2H, br), 3.21 (2H, t, J=4.5 Hz), 3.60 (3H, s), 3.67 (2H, t, J=4.5 Hz), 4.59 (2H, d, J=6 Hz), 7.05 (2H, t, J=8.5 Hz), 7.32 (2H, dd, J=8.3, 5.5 Hz), 7.81 (1H, t, J=5.5 Hz), 12.07 (1H, s). ¹³C NMR (CDCl₃, 125.8 Hz) δ: 13.16, 31.59, 32.29, 42.53, 61.76, 65.27, 81.51, 115.82, 115.99, 123.05, 129.50, 129.56, 133.15, 133.18, 147.69, 149.22, 159.39, 161.50, 163.46, 168.51. HRMS (M+H) calcd for C₁₉H₂₃FN₃O₅: 392.1622. found 392.1626. Anal. calcd for C₁₉H₂₂N₃O₅F: C, 58.30; H, 5.66; N, 10.73. found: C, 58.19; H, 5.65; N, 10.69.

### EXAMPLE 13

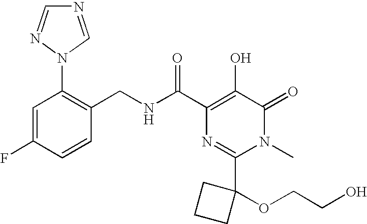

N-(4-Fluoro-2-(1H-1,2,4-triazol-1-yl)benzyl)-5-hydroxy-2-(1-(2-hydroxyethoxy)cyclobut-yl)-1-methyl)-6-oxo-1,6-dihydropyrimidine-4-carboxamide: Yield: 40%, white crystalline powder. ¹H NMR (CDCl₃, 500 MHz) δ: 1.74 (1H, m), 1.83 (1H, br), 1.99 (1H, m), 2.42 (2H, dd, J=10, 21 Hz), 2.83 (2H, br), 3.21 (2H, t, J==4.5 Hz), 3.59 (3H, s), 3.67 (2, t, J=4.5 Hz), 4.47 (2H, d, J=6.7 Hz), 7.11 (1H, dd, J=8.5, 1.3 Hz), 7.22 (1H, dt, J=2.3, 8.3 Hz), 7.72 (1H, dd, J=8.6, 5.8 Hz), 8.14 (1H, s), 8.46 (1H, s), 8.91 (1H, t, J=6.4 Hz), 12.05 (1H, s). ¹³C NMR (CDCl₃, 125.8 Hz) δ: 13.04, 31.61, 32.25, 39.25, 61.76, 65.24, 81.62, 112.26, 112.46, 116.95, 117.11, 124.33, 128.57, 128.60 34.44, 134.52, 136.91, 136.99, 143.97, 147.58, 148.94, 152.88, 159.48, 161.22, 163.21, 168.10. HRMS (M+H) calcd for C₂₁H₂₄FN₆O₅: 459.1792. found: 459.1772. Anal. calcd for C₂H₂₃N₆O₅F: C, 55.01; H, 5.05; N, 18.33. found: C, 54.65; H, 4.74; N, 17.99.

### EXAMPLE 14

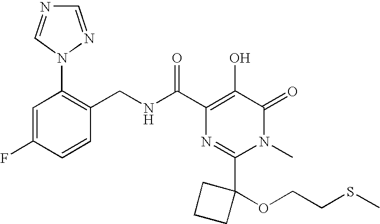

N-(4-Fluoro-2-(1H-1,2,4-triazol-1-yl)benzyl)-5-hydroxy-1-methyl-2-(1-(2-(methylthio)-ethoxy)cyclobutyl)-6-oxo-1,6-dihydropyrimidine-4-carboxamide. ¹H NMR (500 MHz, CDCl₃) δ: 12.06 (1H, br s), 8.93 (1H, t, J=6.56 Hz), 8.50 (1H, s), 8.15 (1H, s), 7.72 (1H, dd, J=7 J=8.55, 6.10 Hz), 7.22 (1H, td, J=8.24, 2.75 Hz), 7.12 (1H, dd, J=8.55, 2.44 Hz), 4.46 (2H, d, J=7.02 Hz), 3.61 (3H, s), 3.27 (2H, t, J=6.41 Hz), 2.77-2.86 (2H, br), 2.58 (2H, t, J=6.56 Hz), 2.37-2.46 (2H, m), 2.04-2.06 (3H, m), 1.95-2.03 (1H, m), 1.67-1.76 (1H, m); ¹³C NMR (126 MHz, CDCl₃) δ; 168.11, 163.22, 161.22, 159.50, 152.73, 149.04, 147.60, 143.94, 136.97, 136.89, 134.53, 134.46, 128.63, 128.60, 124.32, 117.17, 117.01, 112.46, 112.26, 81.59, 63.33, 39.17, 34.16, 32.33 31.59, 16.34, 13.10. HRMS (M+H) calcd for C₂₂H₆N₆O₄FS: 489.1720. found: 489.1723.

### EXAMPLE 15

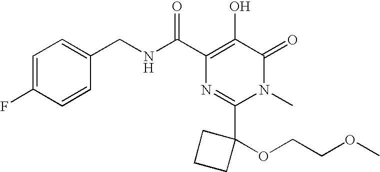

N-(4-Fluorobenzyl)-5-hydroxy-2-(1-(2-methoxyethoxy)cyclobutyl)-1-methyl-6-oxo-1,6-dihydropyrimidine-4-carboxamide: ¹H NMR (500 MHz, CDCl₃) δ: 12.07 (1H, s), 7.81 (1H, t, J=5.80 Hz), 7.32 (2H, dd, J=8.39, 5.34 Hz), 7.05 (2H, t, J=8.70 Hz), 4.59 (2H, d, J=6.41 Hz), 3.60 (3H, s), 3.39-3.43 (2H, m), 3.29 (3H, s), 3.22-3.27 (2H, m), 2.66 (2H, t, J=12.67 Hz), 2.35-2.42 (2H, m), 1.92-2.00 (1H, m), 1.61-1.71 (1H, m); ¹³C NMR (126 MHz, CDCl₃) δ: 168.56, 163.46, 161.49, 159.44, 149.45, 147.71, 133.19, 129.55, 129.49, 123.98, 115.99, 115.82, 81.50, 71.57, 63.44, 59.14, 42.51, 32.32, 31.47, 13.12; HRMS (M+H) calcd for C₂₀H₂₅N₃O₅F: 406.1778. found: 406.1794. Elem. Anal. calcd for C₂₀H₂₄N₃O₅F: C, 59.25; H, 5.97; N, 10.37; F, 4.69. found: C, 59.24; H, 6.10; N, 10.53; F, 4.89.

### EXAMPLE 16

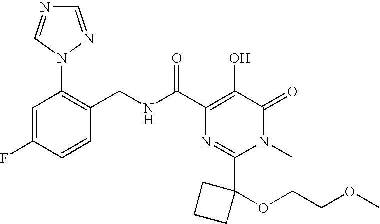

N-(4-Fluoro-2-(1H-1,2,4-triazol-1-yl)benzyl)-5-hydroxy-2-(1-(2-methoxyethoxy)cyclobut-yl)-1-methyl-6-oxo-1,6-dihydropyrimidine-4-carboxamide: ¹H NMR (500 MHz, CDCl₃) δ: 12.01 (1H, br), 8.88 (H, t, J=6.41 Hz), 8.59 (1H, s), 8.20 (1H, s), 7.73 (1H, dd, J=8.70, 5.95 Hz), 7.21-7.25 (1H, m), 7.13 (1H, dd, J=8.55, 2.44 Hz), 4.46 (2H, d, J=6.71 Hz), 3.60 (3H, s), 3.39-3.44 (2H, m), 3.30 (3H, s), 3.22-3.27 (2H, m), 2.80 (2H, br), 2.39-2.47 (2H, m), 1.93-2.06 (1H, m), 1.65-1.77 (14H, m); ¹³C NMR (126 MHz, CDCl₃) δ: 168.21, 162.69, 160.68, 158.54, 150.96, 147.55, 138.56, 138.50, 135.15, 135.09, 132.72, 132.69, 124.57, 120.38, 120.22, 116.84, 116.64, 82.37, 64.39, 40.71, 40.22, 37.58, 32.09, 28.59, 12.77; HRMS (ESI) calcd for C₂₁H₂₆N₄O₆FS: 481.1557. found: 481.1555. Elem. Anal. calcd for C₂₁H₂₅N₄O₆FS: C, 52.49; H, 5.24; N, 11.66; F, 3.95; S, 6.67. found: C, 52.22; H, 5.09; N, 11.43; F, 3.93; S, 6.60.

### EXAMPLE 17

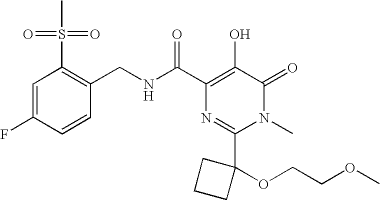

N-(4-Fluoro-2-(methylsulfonyl)benzyl)-5-hydroxy-2-(1-(2-methoxyethoxy)cyclobutyl)-1-methyl-6-oxo-1,6-dihydropyrimidine-4-carboxamide; ¹H NMR (500 MHz, CDCl₃) δ: 11.84 (1H, s), 8.61 (1H, t, J=6.56 Hz), 7.71-7.77 (2H, m), 7.34 (1H, td, J=8.01, 2.59 Hz), 4.83 (2H, d, J=6.71 Hz), 3.59 (3H, s), 3.38-3.41 (2H, m), 3.28 (3H, s), 3.22-3.26 (2H, n), 3.17 (3H, s), 2.70-2.77 (2H, m), 2.36-2.43 (2H, m), 1.92-2.00 (1H, m), 1.62-1.72 (1H, m); ¹³C NMR (126 MHz, CDCl₃) δ: 168.41, 163.16, 161.14, 159.43, 149.58, 147.55, 140.87, 140.83, 135.27, 135.21, 132.90, 132.87, 124.07, 121.61, 121.45, 117.56, 117.36, 81.65, 71.61, 63.37, 59.01, 45.15, 40.25, 32.22, 31.47, 13.10; HRMS (M+H) calcd for C₂₁H₂₇N₃O₇FS: 484.1554. found: 484.1555. Elem. Anal. calcd for C₂₁H₂₆N₃O₇FS: C, 52.16; H, 5.42; N, 8.69. found: C, 51.97; H, 5.33; N, 8.60.

### EXAMPLE 18

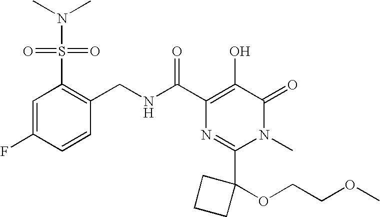

¹H NMR (500 MHz, CDCl₃) δ: 11.97 (1H, s), 8.66 (1H, t, J=6.71 Hz), 7.71 (1H, dd, J=8.39, 5.34 Hz), 7.50 (1H, dd, J=8.39, 2.59 Hz), 7.25-7.30 (1H, m), 4.81 (2H, d, J=7.02 Hz), 3.59 (3H, s), 3.38-3.42 (2H, m), 3.28 (3H, s), 3.22-3.26 (2H, m), 2.90 (6H, s), 2.74 (2H, br), 2.35-2.43 (2H, m), 1.90-1.99 (1H, m), 1.62-1.72 (1H, m); ¹³C NMR (126 MHz, CDCl₃) δ: 168.25, 162.72, 160.71, 159.58, 149.39, 147.49, 138.49, 138.44, 135.34, 135.28, 132.73, 132.70, 124.27, 120.43, 120.26, 116.92, 116.73, 81.61, 71.61, 63.34, 59.08, 40.29, 37.60, 32.29, 13.08; HRMS (M+H) calcd for C₂₂H₃₀N₄O₇FS: 513.1819. found: 513.1827. Elem. Anal. calcd for C₂₂H₃₀N₄O₇FS: C, 51.55; H, 5.70; N, 10.93; S, 6.25; F, 3.70. found: C, 51.37; H, 5.49; N, 10.66; S, 6.24; F, 4.06.

We claim:

1. A compound of Formula I 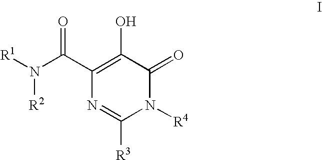
wherein:
R¹ is (Ar¹)alkyl, (Ar¹)(CON(R¹⁰)(R¹¹))alkyl, (Ar¹)(CO₂R¹⁶)alkyl, (Ar¹)hydroxyalkyl, or (Ar¹)oxyalkyl;
R² is hydrogen, alkyl, hydroxy, or alkoxy;
R³ is C(R¹⁷)(R¹⁸)(R¹⁹);
R⁴ is allyl;
R⁵ is hydrogen, halo, hydroxy, cyano, alkyl, cycloalkyl, C₅₋₇cycloalkenyl, haloalkyl, alkoxy, alkylthio, haloalkoxy, N(R¹⁰)(R¹¹), NHAr², N(R⁸)SO₂R⁹, N(R⁸)COR⁹, N(R⁸)CO₂R⁹, OCOR⁹, OCO₂R⁹, OCON(R¹⁰)(R¹¹), OCH₂CO₂R⁹, OCH₂CON(R¹⁰)(R¹¹), COR⁸, CO₂R⁸, CON(R¹⁰)(R¹¹), SOR⁹, S(═N)R⁹, SO₂R⁹, SO₂N(R⁸)(R⁸), PO(OR⁸)₂, C₂₋₄(R¹⁴)alkynyl, R¹⁵, Ar², or Ar³;
R⁶ is hydrogen, halo, hydroxy, cyano, alkyl, alkoxy, haloalkyl, haloalkoxy, or N(R⁸)(R⁸);
R⁷ is hydrogen, halo, hydroxy, cyano, alkyl, alkoxy, haloalkyl, haloalkoxy, or N(R⁸)(R⁸);
R⁸ is hydrogen, alkyl, or cycloalkyl;
R⁹ is allyl or cycloalkyl;
R¹⁰ is hydrogen, alkyl, hydroxyalkyl, alkoxyalkyl or dialkylaminoalkyl;
R¹¹ is hydrogen, alkyl, hydroxyalkyl, alkoxyalkyl or dialkylaminoalkyl; or
N(R¹⁰)(R¹¹) taken together is azetidinyl, pyrrolidinyl, (R¹²)-piperidinyl, N—(R¹³)-piperazinyl, morpholinyl, thiomorpholinyl, or dioxothiazinyl;
R¹² is hydrogen, alkyl, or hydroxyalkyl;
R¹³ is hydrogen, alkyl, cycloalkyl, COR⁸, or CO₂R⁸;
R¹⁴ is hydrogen, hydroxy, N(R⁸)(R⁸), SO₂R⁹, OSO₂R⁹, or dioxothiazinyl;
R¹⁵ is azetidinonyl, pyrrolidinonyl, valerolactamyl, caprolactamyl, maleimido, oxazolidonyl, or dioxothiazinyl, and is substituted with 0-1 substituents selected from the group consisting of hydroxymethyl, acetoxymethyl, and aminomethyl;
R¹⁶ is independently hydrogen or alkyl;
or two R¹⁶'s taken together are CH₂CH₂, CH₂CH₂CH₂, CH₂CH₂CH₂CH₂, CH₂CH₂CH₂CH₂CH₂, CH₂CH₂CH₂CH₂CH₂CH₂, OCH₂CH₂, CH₂OCH₂, OCH₂CH₂CH₂, CH₂OCH₂CH₂, OCH₂CH₂CH₂CH₂, CH₂OCH₂CH₂CH₂, CH₂CH₂OCH₂CH₂, OCH₂CH₂CH₂CH₂CH₂, CH₂OCH₂CH₂CH₂CH₂, CH₂CH₂OCH₂CH₂CH₂, N(R⁸)CH₂CH₂, CH₂N(R⁸)CH₂, N(R⁸)CH₂CH₂CH₂, CH₂N(R⁸)CH₂CH₂, N(R⁸)CH₂CH₂CH₂CH₂, CH₂N(R⁸)CH₂CH₂CH₂, CH₂CH₂N(R⁸)CH₂CH₂, N(R⁸)CH₂CH₂CH₂CH₂CH₂, CH₂N(R⁸)CH₂CH₂CH₂CH₂ or CH₂CH₂N(R⁸)CH₂CH₂CH₂, provided that the two R¹⁶'s are attached to a common carbon atom;
R¹⁷ is alkyl;
R¹⁸ is alkyl;
or R¹⁷ and R¹⁸ taken together with the carbon to which they are attached is C₃₋₇cycloalkylene, a 4-7-membered cyclic ether, or a 4-7-membered cyclic thioether;
R¹⁹ is alkyl, hydroxyalkyl, alkylthioalkyl, alkoxy, alkoxyalkoxy, or alkylthioalkoxy; 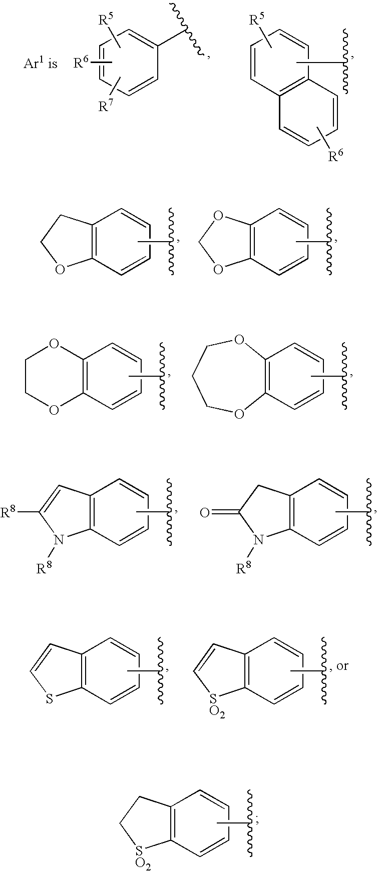
Ar² is tetrazolyl, triazolyl, oxadiazolyl, thiadiazolyl, pyrazolyl, imidazolyl, oxazolyl, thiazolyl, isoxazolyl, isothiazolyl, furanyl, thienyl, pyrrolyl, pyrimidinyl, pyrazinyl, pyridinyl, hydroxypyridinyl, quinolinyl, isoquinolinyl, or indolyl, and is substituted with 0-2 substituents selected from the group consisting of halo, cyano, benzyl, alkyl, alkoxy, N(R¹⁰)(R¹¹), CON(R¹⁰)(R¹¹), CO₂R⁸, CONHSO₂N(R⁸)(R⁸), CONHSO₂N(R⁸)(phenyl), and CONHSO₂N(R⁸)(halophenyl); and
Ar³ is phenyl substituted with 0-2 substituents selected from the group consisting of halo, cyano, hydroxy, alkyl, alkoxy, alkoxymethyl, haloalkyl, haloalkoxy, N(R¹⁰)(R¹¹), CON(R⁸)(R⁸), and CH₂N(R¹⁰)(R¹¹), or is dioxolanylphenyl;
or a pharmaceutically acceptable salt thereof.

2. A compound of claim 1 where R¹ is (Ar¹)alkyl.

3. A compound of claim 1 where R¹ is

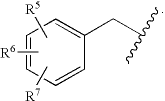

4. A compound of claim 1 where R¹ is 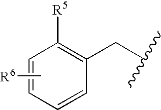
and R⁵ is other than hydrogen and halo.

5. A compound of claim 1 where R¹ is

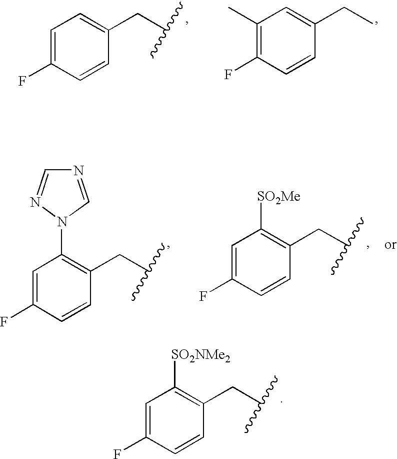

6. A compound of claim 1 where R² is hydrogen.

7. A compound of claim 1 where R³ is

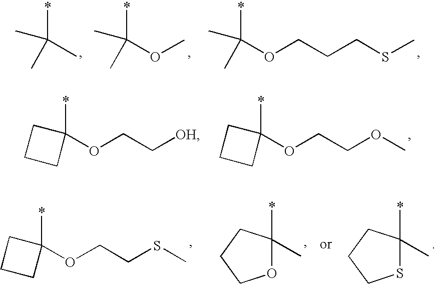

8. A compound of claim 1 where R⁴ is methyl.

9. A compound of claim 1 where R⁵ is R¹⁵.

10. A compound of claim 1 where R⁵ is Ar².

11. A compound of claim 10 where Ar² is tetrazolyl, triazolyl, oxadiazolyl, thiadiazolyl, pyrazolyl, imidazolyl, oxazolyl, thiazolyl, isoxazolyl, isothiazolyl, furanyl, thienyl, or pyrrolyl, and is substituted with 0-2 substituents selected halo and alkyl.

12. A compound selected from the group consisting of
N-(4-Fluorobenzyl)-5-hydroxy-2-(2-methoxypropan-2-yl)-1-methyl-6-oxo-1,6-dihydropyrimidine-4-carboxamide;
N-(4-Fluoro-3-methylbenzyl)-5-hydroxy-2-(2-methoxypropan-2-yl)-1-methyl-6-oxo-1,6-dihydropyrimidine-4-carboxamide;
2-tert-Butyl-5-hydroxy-6-oxo-1,6-dihydro-pyrimidine-4-carboxylic acid 4-fluoro-benzylamide;
2-tert-Butyl-5-hydroxy-1-methyl-6-oxo-1,6-dihydro-pyrimidine-4-carboxylic acid 4-fluoro-benzylamide
N-(4-Fluorobenzyl)-5-hydroxy-1-methyl-2-(2-methyl-6-(methylthio)hexan-2-yl)-6-oxo-1,6-dihydropyrimidine-4-carboxamide;
N-(4-Fluorobenzyl)-5-hydroxy-1-methyl-2-(2-methyl-tetrahydrothiophen-2-yl)-6-oxo-1,6-dihydropyrimidine-4-carboxamide;
N-(4-Fluoro-2-(1H-1,2,4-triazol-1-yl)benzyl)-5-hydroxy-1-methyl-2-(2-methyl-tetrahydrothiophen-2-yl)-6-oxo-1,6-dihydropyrimidine-4-carboxamide;
N-(4-Fluoro-2-(methylsulfonyl)benzyl)-5-hydroxy-1-methyl-2-(2-methyl-tetrahydrofuran-2-yl)-6-oxo-1,6-dihydropyrimidine-4-carboxamide;
N-(4-Fluorobenzyl)-5-hydroxy-1-methyl-2-(2-methyl-tetrahydrofuran-2-yl)-6-oxo-1,6-dihydropyrimidine-4-carboxamide;
N-(4-Fluoro-2-(1H-1,2,4-triazol-1-yl)benzyl)-5-hydroxy-1-methyl-2-(2-methyl-tetrahydrofuran-2-yl)-6-oxo-1,6-dihydropyrimidine-4-carboxamide;
N-(4-Fluoro-2-(methylsulfonyl)benzyl)-5-hydroxy-1-methyl-2-(2-methyl-tetrahydrofuran-2-yl)-6-oxo-1,6-dihydropyrimidine-4-carboxamide;
5-Hydroxy-2-[1-(2-hydroxy-ethoxy)-cyclobutyl]-1-methyl-6-oxo-1,6-dihydro-pyrimidine-4-carboxylic acid 4-fluoro-benzylamide;
N-(4-Fluoro-2-(1H-1,2,4-triazol-1-yl)benzyl)-5-hydroxy-2-(1-(2-hydroxyethoxy)cyclobut-yl)-1-methyl-6-oxo-1,6-dihydropyrimidine-4-carboxamide;
N-(4-Fluoro-2-(1H-1,2,4-triazol-1-yl)benzyl)-5-hydroxy-1-methyl-2-(1-(2-(methylthio)-ethoxy)cyclobutyl)-6-oxo-1,6-dihydropyrimidine-4-carboxamide;
N-(4-Fluorobenzyl)-5-hydroxy-2-(1-(2-methoxyethoxy)cyclobutyl)-1-methyl-6-oxo-1,6-dihydropyrimidine-4-carboxamide;
N-(4-Fluoro-2-(1H-1,2,4-triazol-1-yl)benzyl)-5-hydroxy-2-(1-(2-methoxyethoxy)cyclobut-yl)-1-methyl-6-oxo-1,6-dihydropyrimidine-4-carboxamide; and
N-(4-Fluoro-2-(methylsulfonyl)benzyl)-5-hydroxy-2-(1-(2-methoxyethoxy)cyclobutyl)-1-methyl-6-oxo-1,6-dihydropyrimidine-4-carboxamide;
or a pharmaceutically acceptable salt thereof.

13. A composition useful for treating HIV infections comprising a therapeutic amount of a compound of claim 1 and a pharmaceutically acceptable carrier.

14. The composition of claim 13 further comprising a therapeutically effective amount at least one other agent used for treatment of AIDS or HIV infection selected from the group consisting of nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier.

15. A method for treating HIV infection comprising administering a therapeutically effective amount of a compound of claim 1, or a pharmaceutically acceptable salt thereof, to a patient in need thereof.

16. The method of claim 15 further comprising administering a therapeutically effective amount of at least one other agent used for treatment of AIDS or HIV infection selected from the group consisting of nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.
